SEQUENCE LISTING (128 sequences)

SEQ ID NO: 1 - DNA, 20 bp, Artificial Sequence
  PRIMER
cccttctcagcggtgtcatc

SEQ ID NO: 2 - DNA, 17 bp, Artificial Sequence
  PRIMER
acagaccgtctgggcgc

SEQ ID NO: 3 - DNA, 18 bp, Artificial Sequence
  PRIMER
  misc_feature (1)..(1): t at position 1 is VIC-t
  misc_feature (18)..(18): t at position 18 is t-MGBNFQ
ttcgcgtttccgcaagat

SEQ ID NO: 4 - DNA, 19 bp, Artificial Sequence
  PRIMER
ctgtgtccacctgcattcg

SEQ ID NO: 5 - DNA, 19 bp, Artificial Sequence
  PRIMER
gagaccgggcactcactgt

SEQ ID NO: 6 - DNA, 21 bp, Artificial Sequence
  PRIMER
  misc_feature (1)..(1): t at position 1 is 6FAM-t
  misc_feature (21)..(21): c at position 21 is c-BHQ1
tcagtcctgccatgagcaggc

SEQ ID NO: 7 - DNA, 20 bp, Artificial Sequence
  PRIMER
gggatggaaagtcacgtcct

SEQ ID NO: 8 - DNA, 20 bp, Artificial Sequence
  PRIMER
agagactgggacagcgtcaa

SEQ ID NO: 9 - DNA, 22 bp, Artificial Sequence
  PRIMER
  misc_feature (1)..(1): c at position 1 is 6FAM-c
  misc_feature (22)..(22): a at position 22 is a-BHQ1
cagaatctcagcagctgggcca

SEQ ID NO: 10 - DNA, 20 bp, Artificial Sequence
  PRIMER
aggatggggacttttccttt

SEQ ID NO: 11 - DNA, 20 bp, Artificial Sequence
  PRIMER
tcctcatcttcagctgttgg

SEQ ID NO: 12 - DNA, 24 bp, Artificial Sequence
  PRIMER
  misc_feature (1)..(1): c at position 1 is 6FAM-c
  misc_feature (24)..(24): c at position 24 is c-BHQ1
cattcaggattctgggcttgctcc

SEQ ID NO: 13 - DNA, 20 bp, Artificial Sequence
  PRIMER
tgccaattgcctactgtttg

SEQ ID NO: 14 - DNA, 21 bp, Artificial Sequence
  PRIMER
agcaacagtggaaggagagaa

SEQ ID NO: 15 - DNA, 29 bp, Artificial Sequence
  PRIMER
  misc_feature (1)..(1): g at position 1 is 6FAM-g
  misc_feature (29)..(29): c at position 29 is c-BHQ1
gacaacataaccaagtggggcttattttc

SEQ ID NO: 16 - DNA, 21 bp, Artificial Sequence
  PRIMER
tgaagggacctcagtagttgg

SEQ ID NO: 17 - DNA, 20 bp, Artificial Sequence
  PRIMER
tgacaggcatggttcttctg

SEQ ID NO: 18 - DNA, 17 bp, Artificial Sequence
  PRIMER
  misc_feature (1)..(1): c at position 1 is 6FAM-c
  misc_feature (17)..(17): g at position 17 is g-MGBNFQ
ctgactttctagcaagg

SEQ ID NO: 19 - DNA, 24 bp, Artificial
  PRIMER
tagaggcaggggctctaaaagttc

SEQ ID NO: 20 - DNA, 22 bp, Artificial
  PRIMER
tgtggttgcaaggctgatttcc

SEQ ID NO: 21 - DNA, 18 bp, Artificial
  PRIMER
agtggcatggcagcaggg

SEQ ID NO: 22 - DNA, 22 bp, Artificial
  PRIMER
caccttccagatgggaatgcac

SEQ ID NO: 23 - DNA, 26 bp, Artificial
  PRIMER
gctgaataatagcgtaagggacaggc

SEQ ID NO: 24 - DNA, 26 bp, Artificial
  PRIMER
ctcatggcacccatataaaaagctcc

SEQ ID NO: 25 - DNA, 24 bp, Artificial
  PRIMER
aaacaccctgatttcgtgtgcagg

SEQ ID NO: 26 - DNA, 28 bp, Artificial
  PRIMER
gatttgaggaacctcctttgagagacac

SEQ ID NO: 27 - DNA, 30 bp, Artificial
  PRIMER
gagtgatttgtatcatataggcagcccttc

SEQ ID NO: 28 - DNA, 34 bp, Artificial
  PRIMER
ttacaactggttactccagtagaagtatgtaggc

SEQ ID NO: 29 - DNA, 28 bp, Artificial
  PRIMER
agcagagttagtttagcaaaatgtcccc

SEQ ID NO: 30 - DNA, 27 bp, Artificial
  PRIMER
ggtgattatactttccacctgctcatc

SEQ ID NO: 31 - DNA, 32 bp, Artificial
  PRIMER
acaggtaggtgggtaaatatgaaacttagctg

SEQ ID NO: 32 - DNA, 26 bp, Artificial
  PRIMER
gtgtgctcggagaagagctctaattg

SEQ ID NO: 33 - DNA, 22 bp, Artificial
  PRIMER
atggaaagcagcagccaaactc

SEQ ID NO: 34 - DNA, 20 bp, Artificial
  PRIMER
cataagggctgccatgtgcg

SEQ ID NO: 35 - DNA, 29 bp, Artificial
  PRIMER
ttcaacagaaatgtaattgcttacatgag

SEQ ID NO: 36 - DNA, 27 bp, Artificial
  PRIMER
ctgagttatgtatagccactttggccg

SEQ ID NO: 37 - DNA, 24 bp, Artificial
  PRIMER
aagggttcaatgttcaaaggcatc

SEQ ID NO: 38 - DNA, 25 bp, Artificial
  PRIMER
gcaaaggatctttactgagccaacc

SEQ ID NO: 39 - DNA, 31 bp, Artificial
  PRIMER
cattgttcttaatattgttatcaagggtctc

SEQ ID NO: 40 - DNA, 34 bp, Artificial
  PRIMER
cctaaatcttaaataagtttggtaggaaaaaggg

SEQ ID NO: 41 - DNA, 24 bp, Artificial
  PRIMER
ttggaatggcttacatcttctgtg

SEQ ID NO: 42 - DNA, 27 bp, Artificial
  PRIMER
gtaaagggtctttgtaaatttgggtgg

SEQ ID NO: 43 - DNA, 24 bp, Artificial
  PRIMER
ggctggatgactagagcctacatc

SEQ ID NO: 44 - DNA, 22 bp, Artificial
  PRIMER
acaggtgataaaagcccgagcc

SEQ ID NO: 45 - DNA, 25 bp, Artificial
  PRIMER
caacaagcatgcaaacatcagtgtc

SEQ ID NO: 46 - DNA, 33 bp, Artificial
  PRIMER
agaagaatatttaaactccttattgtggcatac

SEQ ID NO: 47 - DNA, 21 bp, Artificial
  PRIMER
aaagttccccctttggggagg

SEQ ID NO: 48 - DNA, 32 bp, Artificial
  PRIMER
aaagaaaatctgttctggagattcgtatattg

SEQ ID NO: 49 - DNA, 28 bp, Artificial
  PRIMER
gagtgcttaagatcctgatatgagtggc

SEQ ID NO: 50 - DNA, 20 bp, Artificial
  PRIMER
aacctctgccccaacccacc

SEQ ID NO: 51 - DNA, 23 bp, Artificial
  PRIMER
ccttgggaaaggtttagggagcg

SEQ ID NO: 52 - DNA, 21 bp, Artificial
  PRIMER
tgcactgcaatgccacctttg

SEQ ID NO: 53 - DNA, 19 bp, Artificial
  PRIMER
tccctaccagccaaggggg

SEQ ID NO: 54 - DNA, 24 bp, Artificial
  PRIMER
aatgcctttcccagtttcctgtcc

SEQ ID NO: 55 - DNA, 24 bp, Artificial
  PRIMER
aggagagggtcaaccaccataagc

SEQ ID NO: 56 - DNA, 18 bp, Artificial
  PRIMER
caggccctggaagtcccc

SEQ ID NO: 57 - DNA, 30 bp, Artificial
  PRIMER
gcaaatatctaatttttcaagatccagccc

SEQ ID NO: 58 - DNA, 24 bp, Artificial
  PRIMER
aaaaacaaataaacaatggtgccc

SEQ ID NO: 59 - DNA, 27 bp, Artificial
  PRIMER
caaccttcaaacttttgggatagaagc

SEQ ID NO: 60 - DNA, 32 bp, Artificial
  PRIMER
gttccaagagattaaaccgataagaatacctg

SEQ ID NO: 61 - DNA, 31 bp, Artificial
  PRIMER
cagtaggacaatgacaaagtaggcttagcag

SEQ ID NO: 62 - DNA, 24 bp, Artificial
  PRIMER
gaccacagcacattcaacacatcc

SEQ ID NO: 63 - DNA, 21 bp, Artificial
  PRIMER
ccatggcattttatggctccc

SEQ ID NO: 64 - DNA, 30 bp, Artificial
  PRIMER
agaaaacagctatatggtggttgaactatg

SEQ ID NO: 65 - DNA, 29 bp, Artificial
  PRIMER
ggtttaaactatttccaggtggaagaggc

SEQ ID NO: 66 - DNA, 24 bp, Artificial
  PRIMER
attcccaccagctcccttcaccag

SEQ ID NO: 67 - DNA, 23 bp, Artificial
  PRIMER
tttctcccttcctccaaacacgc

SEQ ID NO: 68 - DNA, 22 bp, Artificial
  PRIMER
gaccccaaggagattgctgacc

SEQ ID NO: 69 - DNA, 28 bp, Artificial
  PRIMER
cctggcttccttatcaaactcacttaac

SEQ ID NO: 70 - DNA, 22 bp, Artificial
  PRIMER
gatcgagcagaatgccaccaac

SEQ ID NO: 71 - DNA, 28 bp, Artificial
  PRIMER
tctgtagagtgtgccagttagtacagcc

SEQ ID NO: 72 - DNA, 33 bp, Artificial
  PRIMER
ttcttctcctatatacctgctaatgctcagttc

SEQ ID NO: 73 - DNA, 31 bp, Artificial
  PRIMER
tctctatgccaataaacaaattgaaattaac

SEQ ID NO: 74 - DNA, 28 bp, Artificial
  PRIMER
ctaaatgccatatttttgtatggagggc

SEQ ID NO: 75 - DNA, 27 bp, Artificial
  PRIMER
ttcctcttcccgtgaactctaattttc

SEQ ID NO: 76 - DNA, 33 bp, Artificial
  PRIMER
ccaatcattttacatttgtaactcaactacgtc

SEQ ID NO: 77 - DNA, 24 bp, Artificial
  PRIMER
aacaatgcatcactaccaccaccc

SEQ ID NO: 78 - DNA, 26 bp, Artificial
  PRIMER
cgctccatatcaaagagtaagcaatc

SEQ ID NO: 79 - DNA, 31 bp, Artificial
  PRIMER
cacctatttgaggatacagaccaaacctacc

SEQ ID NO: 80 - DNA, 29 bp, Artificial
  PRIMER
ctttctagagctcatcttctaccaagggc

SEQ ID NO: 81 - DNA, 25 bp, Artificial
  PRIMER
ttacacaacagagcctttctccggc

SEQ ID NO: 82 - DNA, 34 bp, Artificial
  PRIMER
agttctttctaataaataaccaataaatcaaacc

SEQ ID NO: 83 - DNA, 22 bp, Artificial
  PRIMER
aaatatggggccatggcattcg

SEQ ID NO: 84 - DNA, 25 bp, Artificial
  PRIMER
ccacatcccccacatacacacatac

SEQ ID NO: 85 - DNA, 23 bp, Artificial
  PRIMER
agaccacttgccaaaactcccag

SEQ ID NO: 86 - DNA, 30 bp, Artificial
  PRIMER
tctgcccaataatctagtaggcatccagtc

SEQ ID NO: 87 - DNA, 24 bp, Artificial
  PRIMER
aaccatataaccactgggccaacc

SEQ ID NO: 88 - DNA, 34 bp, Artificial
  PRIMER
aaaacacaaaatatacaattataataccccctcc

SEQ ID NO: 89 - DNA, 19 bp, Artificial
  PRIMER
tcccactgcatcaccccag

SEQ ID NO: 90 - DNA, 29 bp, Artificial
  PRIMER
atgcattctagaacaatgaacatgggctg

SEQ ID NO: 91 - DNA, 27 bp, Artificial
  PRIMER
aatatggggtgaggatagaggggtgtg

SEQ ID NO: 92 - DNA, 19 bp, Artificial
  PRIMER
gaagcccgtccagtcctgc

SEQ ID NO: 93 - DNA, 31 bp, Artificial
  PRIMER
tttatcataaaaagtgtagggcaagtagatg

SEQ ID NO: 94 - DNA, 23 bp, Artificial
  PRIMER
cagatgtgacaaagtctggaagg

SEQ ID NO: 95 - DNA, 24 bp, Artificial
  PRIMER
tgccaacaagctgagtccaaagac

SEQ ID NO: 96 - DNA, 30 bp, Artificial
  PRIMER
attttcttaaataacctttaagcgtgtgtc

SEQ ID NO: 97 - DNA, 26 bp, Artificial
  PRIMER
aacacacatgccctaagtgtttctcc

SEQ ID NO: 98 - DNA, 33 bp, Artificial
  PRIMER
cacacacactacatacttggatatatacgtttc

SEQ ID NO: 99 - DNA, 26 bp, Artificial
  PRIMER
gccctcagatgattattggtgaagtg

SEQ ID NO: 100 - DNA, 18 bp, Artificial
  PRIMER
aaccagggcaagacccgc

SEQ ID NO: 101 - DNA, 26 bp, Artificial
  PRIMER
tttgcaagtttatgaatgctgagatg

SEQ ID NO: 102 - DNA, 30 bp, Artificial
  PRIMER
ttgatccttggtcttagtttagcctattcc

SEQ ID NO: 103 - DNA, 24 bp, Artificial
  PRIMER
tttgtgttggtttttgagggcaac

SEQ ID NO: 104 - DNA, 21 bp, Artificial
  PRIMER
cacatcccacccaggcagttc

SEQ ID NO: 105 - DNA, 21 bp, Artificial
  PRIMER
taccttctcagcaaggccatc

SEQ ID NO: 106 - DNA, 28 bp, Artificial
  PRIMER
gcatccacaaagtcttccaaactgtagg

SEQ ID NO: 107 - DNA, 29 bp, Artificial
  PRIMER
gataggatctgatcagaaatgcactcagc

SEQ ID NO: 108 - DNA, 24 bp, Artificial
  PRIMER
caatccaagattcgaaaatgcacg

SEQ ID NO: 109 - DNA, 22 bp, Artificial
  PRIMER
catgagcagcttcctacctgcc

SEQ ID NO: 110 - DNA, 21 bp, Artificial
  PRIMER
tgttgcgtcacagagggggag

SEQ ID NO: 111 - DNA, 23 bp, Artificial
  PRIMER
ctggtgctttcattgcggaaaac

SEQ ID NO: 112 - DNA, 34 bp, Artificial
  PRIMER
tttaactacgattaatgatggaatagagaaagac

SEQ ID NO: 113 - DNA, 26 bp, Artificial
  PRIMER
ggtatgaagttgtgaagtagtgccag

SEQ ID NO: 114 - DNA, 22 bp, Artificial
  PRIMER
cagaggagtgtgtgactgcccc

SEQ ID NO: 115 - DNA, 25 bp, Artificial
  PRIMER
agattttaaggaagcactccacccc

SEQ ID NO: 116 - DNA, 23 bp, Artificial
  PRIMER
atcctggccttaggatgaggacg

SEQ ID NO: 117 - DNA, 26 bp, Artificial
  PRIMER
agcatagcgacaacctatgccttctg

SEQ ID NO: 118 - DNA, 29 bp, Artificial
  PRIMER
tgttcaccttcaatatctctccactcttg

SEQ ID NO: 119 - DNA, 26 bp, Artificial
  PRIMER
tcttagtcggagtcatgggtatttgc

SEQ ID NO: 120 - DNA, 28 bp, Artificial
  PRIMER
gctgtgcatggctagatgacttgtactc

SEQ ID NO: 121 - DNA, 28 bp, Artificial
  PRIMER
aggtcagagaagtgactgaaaaatgtgg

SEQ ID NO: 122 - DNA, 22 bp, Artificial
  PRIMER
ggccttggccatagtcaacaag

SEQ ID NO: 123 - DNA, 28 bp, Artificial
  PRIMER
atgttttcataaattaaccctaacccag

SEQ ID NO: 124 - DNA, 30 bp, Artificial
  PRIMER
aaaaggcctaactagtgaacccatgtaatc

SEQ ID NO: 125 - DNA, 18 bp, Artificial
  PRIMER
acttcacccggatgcgcc

SEQ ID NO: 126 - DNA, 21 bp, Artificial
  PRIMER
aaacctctggctcagtgttgg

SEQ ID NO: 127 - DNA, 29 bp, Artificial
  PRIMER
gctaaatagtggcaaagacctcgtctacc

SEQ ID NO: 128 - DNA, 27 bp, Artificial
  PRIMER
ggactctcctgacttttcaatccaacc

(19) United States
(12) Patent Application Publication
(10) Pub. No.: US 2007/0172853 A1
(43) Pub. Date: Jul. 26, 2007

(54) Use of deletion polymorphisms to predict, prevent, and manage histoincompatibility

(75) Inventors: Steven A. McCarroll, Cambridge, MA (US); David M. Altshuler, Brookline, MA (US)

Correspondence Address: CLARK & ELBING LLP, 101 FEDERAL STREET, BOSTON, MA, 02110, US

(73) Assignee: The General Hospital Corporation, Boston, MA (US)

(21) Appl. No.: 11/633,336

(22) Filed: Dec. 4, 2006

Related U.S. Application Data
(60) Provisional application No. 60/741,638, filed on Dec. 2, 2005.

(51) Int. Cl. C12Q 1/68

(52) U.S. Cl. 435/6

(57) ABSTRACT

Disclosed herein are methods for predicting the immunocompatibility of two subjects that include determining the presence or absence of one or more deletion variants in the DNA sequence of a gene, where the deletion variant substantially prevents expression of the protein encoded by the gene.

### CROSS REFERENCE TO RELATED APPLICATION

This application claims the benefit of U.S. Provisional Application No. 60/741,638 filed on Dec. 2, 2005, herein incorporated by reference.

### STATEMENT AS TO FEDERALLY FUNDED RESEARCH

The United States Government has a paid-up license in this invention and the right in limited circumstances to require the patent owner to license others on reasonable terms as provided for by the terms of grant number 1U54HG02750 awarded by the National Human Genome Research Institute of the National Institutes of Health.

### BACKGROUND OF THE INVENTION

Organ and bone marrow transplantation are routinely used for the treatment of patients with end-stage disease such as leukemia, liver failure due to hepatitis C, and kidney failure. While the frequency of organ and tissue transplants has increased dramatically over the past decades, histoincompatibility between the transplant recipient and the donor remains a significant barrier to the success of the transplant.

Histocompatibility, also known as immunocompatibility, refers to the compatibility between two individuals or the actual organs or tissues to be transplanted (also known as “grafts”). Consequences of histoincompatibility include graft rejection, also known as host versus graft disease (HVGD) in organ transplant, and graft versus host disease (GVHD), typically associated with bone marrow transplants. In GVHD, immune cells derived from donor hematopoetic stem cells identify host tissue as foreign and mount an immune response against them. In HVGD, host immune cells identify the graft organ as foreign and mount an immune response against it. Both GVHD and HVGD are debilitating conditions and can require patients to be placed on severe immunosuppressive regimens, with attendant complications. Immunocompatibility largely depends on the genetic similarities between donor and recipient and is generally determined by blood typing and by Major Histocompatibility Complex (MHC) typing, which in humans is also referred to as the Human Leukocyte Antigen (HLA) typing. The MHC of humans is a cluster of genes occupying a region located on the sixth chromosome. The strongest antigens of the MHC are separated into two classes—class I and class II. Class I and II MHC molecules are found in nearly every cell in the body and are the major determinants used by the body's immune system for recognition and differentiation of self from non-self. MHC molecules present antigen peptides to the T cells of the immune system and different MHC molecules differ in the efficiency with which they bind sequences of the antigenic peptides and some are better than others at presenting antigens to the immune system. The class I MHC molecules are encoded by three loci—HLA A, HLA B, and HLA C—and class II MHC molecules are encoded by three loci—HLA DR, HLA DP, and HLA DQ. While the number of alleles at each locus varies widely, a person can only inherit two alleles for each HLA locus. The large number of possible combinations at each locus make the genes of the MHC the most polymorphic loci known.

Every person's HLA pattern can be “fingerprinted” through tissue typing. Tissue typing, or HLA matching, is used to measure the pattern of HLA antigens present for a potential transplant donor and recipient and to determine the level of compatibility between them. The more similar the HLA antigen patterns are from the two tissue samples, the less likely it is that the graft will be rejected.

HLA typing has revolutionized the treatment of many end-stage diseases by increasing the success rate of transplantation of bone marrow cells or organs, but graft rejection still occurs with significant frequency even in sibling transplants in which donor and host are perfectly matched for all blood type and HLA antigens. This may be due, at least in part, to the fact that many other histocompatibility antigens have not yet been identified.

Despite the advances in tissue typing and the creation of numerous tissue and organ registries used to screen potential donors and recipients prior to transplantation, the prevalence of life-threatening complications such as graft failure and rejection remains a significant barrier to the overall success of transplantation.

### SUMMARY OF THE INVENTION

The compatibility of bodily tissues with the immune system is a central and unpredictable feature of the etiology of numerous medical conditions, including the rejection of allografts, the development of GVHD and HVGD, spontaneous abortions, and the treatment of many hematologic disorders. Improvements to the methods currently available for screening recipients in need of a transplant against potential donors are necessary to reduce the likelihood of graft rejection, GVHD, and HVGD.

Histoincompatibility is generally believed to be due to genetic differences or polymorphisms between individuals. Because the DNA of any two individuals is known to differ at millions of single-nucleotide polymorphisms (SNP) scattered throughout the human genome, it is often assumed that histoincompatibility results from a large number of small differences between the antigen repertoires of the two individuals. However, we have discovered places in the human genome in which entire segments, ranging from hundreds of base pairs to multi-kilobases of the human genome, are present in some individuals and missing in others. Many of these individual “deletion polymorphisms” or “deletion variants” remove protein-coding sequences from the human genome, and thus result in large changes to an individual's antigen repertoire relative to the changes associated with individual SNPs.

When a deletion variant appears in all copies of the gene in an individual, the result is generally a lack of expression of the gene product in that individual. If an individual does not have the deletion in all copies of the gene, the gene is present and the gene product is generally expressed. As a result, the immune cells of an individual with a deletion variant in all copies of the gene will not have been exposed to this gene or its product, and will tend to recognize the gene product as foreign when it is presented on tissue from another individual. In the context of transplant, this will result in an immune response when the donor and host are not matched, also known as a “deletion mismatch” for the specific deletion polymorphism. For example, in the context of organ transplant, a person having a deletion variant in gene X that results in a lack of expression of gene X that receives a kidney from a donor that does not have a deletion variant in gene X, and is therefore positive for gene X, could mount an immune response against the antigen encoded by gene X and the cells which express it. In the context of bone marrow transplant, immune cells from a donor having a deletion variant in gene X, if transplanted into an individual who is positive for gene X, could mount an immune response against the product of gene X and the cells that express it. In the context of fetal loss, a mother who lacks gene X could miscarry a fetus which is positive for gene X due to an immune response by the mother against the product of gene X.

Several of these common deletion variants are present in genes that are specifically expressed in organs relevant to transplantation and are likely to be determinants used by the body's immune system for recognition and differentiation of self from non-self. If the presence of a deletion resulting in the absence of the antigen is not matched between two subjects for whom immunocompatibility is desired (e.g., a graft donor and a graft recipient), the result is an immune response mounted by the subject having the deletion (i.e., lacking the gene product) against the protein product present in the subject lacking the deletion (i.e., having the expressed protein product). Therefore, these common deletion variants that affect the expression of antigens can be used to screen individuals for immunocompatibility, and used to manage, measure, prevent, and provoke histoincompatibility.

Accordingly, in one aspect, the invention features a method for predicting immunocompatibility of the immune system of a first subject with a cell, tissue, or organ from a second subject that includes the following steps. A biological sample from a first subject and a biological sample from a second subject are obtained and the presence or absence of at least one deletion variant in the DNA sequence of a gene in the first and second biological samples is determined, where the deletion variant substantially prevents expression of an antigen encoded by the gene and where the deletion variant is in a gene selected from the group consisting of UGT2B28, TRY6, LCE3C, PRB1, OR51A2, ORF4F5, GNB1L, MGAM, and MCEE. The deletion variant can be a common deletion variant and can be in anywhere in the gene including the coding region or in a regulatory element of the gene. In one embodiment, the deletion variant is at least 100, 200, 300, 400, 500, 600, 700, 800, 900, or 1000 bp, or 2 kb, 3 kb, 4 kb, 5 kb, 7 kb, 8 kb, 9 kb, 10 kb, 100 kb, 200 kb, 300 kb, 400 kb, 500 kb, 600 kb, 700 kb, 745 kb, 800 kb, 900 kb, or 1000 kb in length. In one embodiment, the deletion variant is between 100 bp and 745 kb in length. In another embodiment, at least two, three, four, five, six, seven, eight, nine, ten or more deletion variants are identified. The presence or absence of the deletion variant can be determined, for example, by polymerase chain reaction, DNA sequencing, sequencing of the whole genome, or a subset thereof, Southern blotting, restriction fragment length polymorphism analysis, microelectrophoresis, sequencing by hybridization, single molecule sequencing, or microarray analysis. The presence or absence of the deletion variant can also be determined indirectly by testing polymorphisms (e.g., SNPs) that are in linkage disequilibrium with deletion polymorphisms or by genotyping polymorphisms (e.g., SNPs) that are inside a deleted region to infer the presence of a deletion that removes the site of the SNP. Preferably, the deletion is in a gene that is normally expressed in the biological sample.

The presence or absence of the at least one deletion variant in the DNA sequence of the gene is then compared between the first and second subjects. The immune system of the first subject is immunocompatible with a cell, tissue, or organ from the second subject if the comparison results in one of the following: (i) the first subject has at least one intact copy of the gene, where the antigen encoded by the gene is expressed or (ii) the second subject has a deletion variant in all copies of the gene, where the deletion variant substantially prevents expression of the antigen encoded by the gene. Based on this comparison, three possible scenarios would predict immunocompatibility between the immune system of the first subject and the cell, tissue, or organ from the second subject: 1) both the first and second subjects have a deletion variant in all copies of the gene, which substantially prevents expression of the antigen, encoded by the gene, in both subjects; 2) both subjects have at least one intact copy of the gene and the antigen encoded by the gene is expressed; and 3) the second subject has a deletion variant in all copies of the gene that substantially prevents expression of the antigen encoded by the gene and the first subject has at least one intact copy of the gene that does not have a deletion variant, in which case, the antigen is expressed.

In one embodiment, the method further includes determining the presence or absence of at least one additional deletion variant in the DNA sequence of a gene in the first and second biological sample where the deletion variant substantially prevents expression of an antigen encoded by the gene and where the at least one additional deletion variant is in a gene selected from the group consisting of UGT2B17, GSTT1, GSTM1, and CYP2A6. The presence or absence of the at least one additional deletion variant in the DNA sequence of the gene is then compared between the first and second subjects. The immune system of the first subject is immunocompatible with a cell, tissue, or organ from the second subject if the comparison results in one of the following: (i) the first subject has at least one intact copy of the gene, where the antigen encoded by the gene is expressed or (ii) the second subject has a deletion variant in all copies of the gene, where the deletion variant substantially prevents expression of the antigen encoded by the gene. Based on this comparison for the additional deletion variant, any of the three possible scenarios described above would predict immunocompatibility between the immune system of the first subject and the cell, tissue, or organ from the second subject. In one desirable combination, the at least one deletion variant is in the UGT2B28 gene and the at least one additional deletion variant is in the UGT2B17 gene. In another desirable combination, the at least one deletion variant is in the UGT2B28 gene and the at least one additional deletion variant is in the GSTT1 or GSTM1 gene, or both.

In a related aspect, the invention features a method for predicting immunocompatibility of the immune system of a first subject with a cell, tissue, or organ from a second subject that includes the following steps. A biological sample from a first subject and a biological sample from a second subject are obtained and the presence or absence of at least one deletion variant antigen in the first and second biological samples is determined, for example, using immunological methods (e.g., ELISA or western blotting based methods). The at least one deletion variant antigen can be a common deletion variant antigen and is preferably one of the following: UGT2B28, TRY6, LCE3C, PRB1, OR51A2, ORF4F5, GNB1L, MGAM, and MCEE. The deletion variant antigen is not an antigen encoded by an MHC, HLA, or Rh factor gene. In one embodiment, at least two, three, four, five, six, seven, eight, nine, ten or more deletion variant antigens are compared.

The presence or absence of the deletion variant antigen is then compared between the first and second subjects. The immune system of the first subject is immunocompatible with a cell, tissue, or organ from the second subject if the comparison results in one of the following: i) the first subject expresses the at least one deletion variant antigen or (ii) the second subject does not express the at least one deletion variant antigen. Based on this comparison, three possible scenarios would predict immunocompatibility between the immune system of the first subject and the cell, tissue, or organ from the second subject: 1) both the first and second subjects express the deletion variant antigen; 2) both the first and second subjects do not express the deletion variant antigen; and 3) the first subject expresses the deletion variant antigen and the second subject does not express the deletion variant antigen.

In one embodiment, the method further includes determining the presence or absence of at least one additional deletion variant antigens selected from the group consisting of UGT2B17, GSTT1, GSTM1, and CYP2A6. The presence or absence of the at least one additional deletion variant antigen is then compared between the first and second subjects. The immune system of the first subject is immunocompatible with a cell, tissue, or organ from the second subject if the comparison results in one of the following: i) the first subject expresses the at least one additional deletion variant antigen or (ii) the second subject does not express the at least one additional deletion variant antigen. Based on this comparison for the additional deletion variant, any of the three possible scenarios described above would predict immunocompatibility between the immune system of the first subject and the cell, tissue, or organ from the second subject. In one desirable combination, the at least one deletion variant antigen is UGT2B28 and the at least one additional deletion variant antigen is UGT2B17. In another desirable combination, the at least one deletion variant antigen is UGT2B28 and the at least one additional deletion variant antigen is GSTT1 or GSTM1, or both.

In a related aspect, the invention also features a method for predicting the immunocompatibility of the immune system of a first subject with a cell, tissue, or organ from a second subject that includes the following steps. A biological sample is obtained from the first subject and second subjects. The presence or absence of one or more deletion variants in the DNA sequence of at least one gene in the biological samples is determined, where the one or more deletion variants substantially prevents the expression of an antigen encoded by the at least one gene. The deletion variant is not in an MHC, Rh factor, or HLA gene. The deletion variant can be a common deletion variant and can be in anywhere in the gene including the coding region or in a regulatory element of the gene. In one embodiment, the deletion variant is at least 100, 200, 300, 400, 500, 600, 700, 800, 900, or 1000 bp, or 2 kb, 3 kb, 4 kb, 5 kb, 7 kb, 8 kb, 9 kb, 10 kb, 100 kb, 200 kb, 300 kb, 400 kb, 500 kb, 600 kb, 700 kb, 745 kb, 800 kb, 900 kb, or 1000 kb in length. In one embodiment, the deletion variant is between 100 bp and 745 kb in length. The presence or absence of the deletion variant can be determined, for example, by polymerase chain reaction, DNA sequencing, Southern blotting, restriction fragment length polymorphism analysis, or microarray analysis. The presence or absence of the deletion variant can also be determined indirectly by testing polymorphisms (e.g., SNPs) that are in linkage disequilibrium with deletion polymorphisms or by genotyping polymorphisms (e.g., SNPs) that are inside a deleted region to infer the presence of a deletion that removes the site of the SNP. Preferably, the deletion is in a gene that is normally expressed in the biological sample. Preferably, the deletion variant is in one of the following genes: UGT2B17, UGT2B28, TRY6, LCE3C, GSTM1, GSTT1, CYP2A6, PRB1, OR51A2, ORF4F5, GNB1L, MGAM, and MCEE.

The presence or absence of the deletion variants is then used to determine the deletion variant pattern for the first and second subjects. The deletion variant pattern is compared between the first and second subjects and the immune system of the first subject is immunocompatible with a cell, tissue, or organ from the second subject if the subjects have a substantially identical deletion variant pattern (e.g., at least 50%, 60%, 70%, 80%, 90%, 95%, 99%, or 100% identical) and the subjects are not immunocompatible if they do not have a substantially identical deletion variant pattern (e.g., less than 50%, 40%, 30%, 20%, 10%, 5%, or less The immune system of the first subject is also immunocompatible with a cell, tissue, or organ from the second subject if the comparison results in one of the following: (i) the first subject has at least one intact copy of at least one gene, where the antigen encoded by the gene is expressed or (ii) the second subject has a deletion variant in all copies of the at least one gene, where the deletion variant substantially prevents expression of the antigen encoded by the gene. Based on this comparison, three possible scenarios would predict immunocompatibility between the immune system of the first subject and the cell, tissue, or organ from the second subject: 1) both the first and second subjects have a deletion variant in all copies of the same gene, which substantially prevents expression of the antigen, encoded by the gene, in both subjects; 2) both subjects have at least one intact copy of the same gene and the antigen encoded by the gene is expressed; and 3) the second subject has a deletion variant in all copies of the same gene that substantially prevents expression of the antigen encoded by the gene and the first subject has at least one intact copy of the same gene that does not have a deletion variant, in which case, the antigen is expressed.

Optionally, the method can further include determining the presence or absence of the antigen encoded by the at least one gene that is not an MHC gene, where the presence or absence of the antigen is used to determine the deletion variant antigen pattern for the first and second subjects. The deletion variant antigen pattern is compared between the first and second subjects and the immune system of the first subject is immunocompatible with a cell, tissue, or organ from the second subject if the subjects have a substantially identical deletion antigen variant pattern (e.g., at least 50%, 60%, 70%, 80%, 90%, 95%, 99%, or 100% identical) and the subjects are not immunocompatible if they do not have a substantially identical deletion variant antigen pattern (e.g., less than 50%, 40%, 30%, 20%, 10%, 5%, or less than 1% identical). The immune system of the first subject is immunocompatible with a cell, tissue, or organ from the second subject if the comparison results in one of the following: (i) the first subject expresses the deletion variant antigen or (ii) the second subject does not express the deletion variant antigen. Based on this comparison, three possible scenarios would predict immunocompatibility between the immune system of the first subject and the cell, tissue, or organ from the second subject: (1) both the first subject and the second subject express the deletion variant antigen, (2) both the first subject and the second subject do not express the deletion variant antigen, or (3) the first subject expresses the deletion variant antigen and the second subject does not express the antigen.

In another aspect, the invention features a method for predicting immunocompatibility of the immune system of a first subject with a cell, tissue, or organ from a second subject that includes the following steps. A biological sample from a first subject and a biological sample from a second subject are obtained and the DNA sequence of the whole genome, or a subset thereof, is determined. The sequence of the whole genome, or subset thereof from the first sample and the second sample are then compared and the presence or absence of at least one deletion mismatch loci is determined. A deletion mismatch loci includes at least one deletion variant in the DNA sequence of a gene, where the deletion variant substantially prevents expression of an antigen encoded by the gene. In one embodiment, the deletion variant is in the DNA sequence of any one or more of the following genes: UGT2B17, UGT2B28, TRY6, LCE3C, GSTM1, GSTT1, CYP2A6, PRB1, OR51A2, ORF4F5, GNB1L, MGAM, and MCEE. The deletion variant can be a common deletion variant and can be in anywhere in the gene including the coding region or in a regulatory element of the gene. In one embodiment, the deletion variant is at least 100, 200, 300, 400, 500, 600, 700, 800, 900, or 1000 bp, or 2 kb, 3 kb, 4 kb, 5 kb, 7 kb, 8 kb, 9 kb, 10 kb, 100 kb, 200 kb, 300 kb, 400 kb, 500 kb, 600 kb, 700 kb, 745 kb, 800 kb, 900 kb, or 1000 kb in length. In one embodiment, the deletion variant is between 100 bp and 745 kb in length. In another embodiment, at least two, three, four, five, six, seven, eight, nine, ten or more deletion mismatch loci are identified. The whole genome, or a subset thereof, can be sequenced using any technique known in the art including, but not limited to, microelectrophoresis, genomic hybridization, single molecule sequencing, or microarray analysis. Preferably, the deletion mismatch is a deletion variant in a gene that is normally expressed in the biological sample. Alternatively or additionally, the sequence of the genome or subset thereof of the first subject can be compared to a reference genome DNA sequence, where the reference genome sequence can be the DNA sequence from a third subject or from a composite of multiple subjects.

The immune system of the first subject is immunocompatible with a cell, tissue, or organ from the second subject if the comparison results in one of the following: (i) the first subject has at least one intact copy of the gene, where the antigen encoded by the gene is expressed or (ii) the second subject has a deletion variant in all copies of the gene, where the deletion variant substantially prevents expression of the antigen encoded by the gene. Based on this comparison, three possible scenarios would predict immunocompatibility between the immune system of the first subject and the cell, tissue, or organ from the second subject: 1) both the first and second subjects have a deletion variant in all copies of the gene, which substantially prevents expression of the antigen, encoded by the gene, in both subjects; 2) both subjects have at least one intact copy of the gene and the antigen encoded by the gene is expressed; and 3) the second subject has a deletion variant in all copies of the gene that substantially prevents expression of the antigen encoded by the gene and the first subject has at least one intact copy of the gene that does not have a deletion variant, in which case, the antigen is expressed.

Each of the above methods can be used alone or in combination to determine immunocompatibility between an organ, tissue, or cell donor and a recipient or between a woman and a potential father, an embryo, or fetus (collectively referred to as “maternal/fetal compatibility”). For organ transplants and maternal/fetal compatibility, the first subject is the organ or tissue recipient or the woman and the second subject is the organ or tissue donor, the prospective father, or the embryo or fetus. In each of these scenarios, the immune system of the recipient would not be newly exposed to the antigen upon transplantation. For bone marrow or peripheral blood transplantation, the first and second subjects are reversed, that is, the first subject is the bone marrow or peripheral blood donor and the second subject is the bone marrow or peripheral blood recipient.

Each of the above methods can further include determining the blood type or the MHC type for the first or second subject. In various embodiments of the above aspects, the first or second biological sample is an organ, or part thereof, a tissue, or a bodily fluid, such as blood, serum, plasma, bone marrow, cerebrospinal fluid, amniotic fluid, urine, saliva, or semen.

In one example of the above aspects, the second subject is in need of a bone marrow or peripheral blood transplant and the first subject is a potential bone marrow or peripheral blood donor and the method is used to determine if the two subjects are a donor/recipient match. In this example, the deletion variant can be identified, for example, in a UGT2B17, UGT2B28, GSTM1, GSTT1, MGAM, or CYP2A6 gene or in the antigen encoded by the any of the genes. In another example, the first subject is an organ or tissue recipient and the second subject is a potential organ or tissue donor and the method is used to determine if the two subjects are a donor/recipient match. For example, the methods can be used to identify a donor/recipient match for a subject in need of a liver transplant where the deletion variant is preferably identified in one or more of the following genes: UGT2B17, UGT2B28, GSTM1, GSTT1, and CYP2A6, or in the antigens encoded by any of the genes. In another example, the methods can be used to identify a donor/recipient match for a subject in need of a kidney transplant where the deletion variant is identified in a UGT2B28, GSTT1 or GSTM1 gene or in the antigens encoded by the genes.

In yet another example of the above aspects, the method is used to predict the immunocompatibility of prospective parents (e.g., where the first subject is a woman and the second subject is a prospective father or a potential sperm donor) or between a woman and an embryo (e.g., an embryo that is conceived by in vitro fertilization) or a pregnant woman and her fetus. Desirably, if the method is used to determine immunocompatibility between a woman and an embryo or fetus, the deletion variant antigen or deletion variant encoding the antigen is normally expressed by the fetal or embryonic cells. For example, the methods can be used to determine compatibility between a woman and an embryo or fetus where the deletion variant is preferably identified in one or more of the following genes: UGT2B28, UGT2B17, or LCE3C, which are expressed in the placenta, or in the antigens encoded by any of the genes.

If, using any of the above methods described herein, the first and second subjects are not immunocompatible, the deletion variant antigen can be administered to the first subject to tolerize the subject to the deletion variant antigen. The deletion variant antigen can be administered by gene therapy or protein therapy.

The methods of the above aspects can also be used to determine histoincompatibility. For example, if the second subject is in need of a bone marrow or peripheral blood transplant and the first subject is a bone marrow or peripheral blood donor, the method can be used to identify the subjects as a donor/recipient match if the first subject is not immunocompatible with the second subject. Such a method can be used, for example, to treat a subject that has a hematologic disorder (e.g., myelodysplastic syndrome, aplastic anemia, sickle cell anemia, metabolic disease, or a blood cell cancer such as Hodgkin's lymphoma, non-Hodgkin's lymphoma, leukemia, and multiple myeloma) and the desired outcome is for the donor's immune cells to attack the diseased cells in the host. For example, if the second subject has a blood cell cancer, the deletion variant is preferably detected in an antigen or in a gene that encodes an antigen that is specifically expressed on the cancer cells in the patient suffering from the blood cell cancer.

The invention also features a kit for deletion variant typing that includes at least one nucleic acid molecule that is complementary to a DNA sequence of at least a portion of a gene selected from the following: UGT2B28, TRY6, LCE3C, PRB1, OR51A2, ORF4F5, GNB1L, MGAM, and MCEE. The kit also includes instructions for the use of the nucleic acid molecule for deletion variant typing. The kit can further include at least one additional nucleic acid molecule that is complementary to the DNA of any one or of the following genes: UGT2B17, GSTT1, GSTM1, and CYP2A6. The nucleic acid molecule can be a primer used for a polymerase chain reaction or a probe that hybridizes to the gene at high stringency.

The invention also features a kit for deletion variant antigen typing that includes at least one binding agent (e.g., an antibody or fragment thereof) that specifically binds at least one antigen encoded by a gene selected from the following: UGT2B28, TRY6, LCE3C, PRB1, OR51A2, ORF4F5, GNB1L, MGAM, and MCEE. The kit can also include at least one binding agent (e.g., ann antibody or fragment thereof) that specifically binds a at least one antigen encoded by a gene selected from the following: UGT2B17, GSTT1, GSTM1, and CYP2A6. The kit also includes instructions for the use of the binding agent (e.g., antibody or fragment thereof) for deletion variant antigen typing.

By “antigen” is meant a polypeptide chain of two or more amino acids regardless of any post-translational modification (e.g., glycosylation or phosphorylation) that stimulates a cellular or humoral immune response.

By “biological sample” is meant a tissue biopsy, cell, bodily fluid (e.g., blood, serum, plasma, semen, urine, saliva, amniotic fluid, or cerebrospinal fluid), organ, or part thereof, or other specimen obtained from a patient or a test subject. Desirably, the biological sample includes nucleic acid molecules or polypeptides or both.

By “cell, tissue, or organ” is meant any cell, tissue or organ from the body or bodily fluid of a subject. Non-limiting examples of organs include kidney, liver, skin, pancreas, heart, lung, muscle, small bowel, hand, cornea, or any part thereof Non-limiting examples of tissues include skin, bone, heart valve, blood, bone marrow, semen, an embryo, and a fetus. Non-limiting examples of cells include red blood cells, white blood cells, stem cells, sperm, egg, embryonic cells, and fetal cells.

By “deletion variant” or “deletion polymorphism” is meant a segment of the genome that is present in some individuals of a species and absent in other individuals of that species. Deletion variants can vary in size from 100, 200, 300, 400, 500, 600, 700, 800, 900, or 1000 bp, or 2 kb, 3 kb, 4 kb, 5 kb, 7 kb, 8 kb, 9 kb, 10 kb, 100 kb, 200 kb, 300 kb, 400 kb, 500 kb, 600 kb, 700 kb, 745 kb, 800 kb, 900 kb, or 1000 kb in length. In one embodiment, the deletion variant is between 100 bp and 745 kb in length. By “common deletion variant” is meant a deletion variant that is seen with a frequency of at least 2%, 3%, 4%, 5%, 6%, 7%, 8%, 9%, or at least 10% in a given population. Most common deletions appear to result from ancestral mutations that have been inherited by descent; their frequency is strongly related to ancestry, and they are in linkage disequilibrium with nearby SNP variants. Desirably, the deletion variant or common deletion variant is a deletion in all copies of the gene that prevents expression of a gene, or prevents expression of an antigen encoded by a gene. Deletion variants can be found in the exons, introns, or the coding region of the gene or in the sequences that control expression of the gene. Examples of protein-encoding genes identified as having common deletion polymorphisms include UGT2B17, UGT2B28, TRY6, LCE3C, GSTM1, GSTT1, CYP2A6, PRB1, OR51A2, ORF4F5, GNB1L, MGAM, and MCEE.

By “a deletion variant in all copies of the gene” or “homozygous deletion” is meant the deletion of all of an individual's potential copies of a DNA locus, which may result from inheritance of a substantially identical deletion variant from both parents; or from the inheritance of different but overlapping deletions from one's parents; or from the combined effect of an inherited deletion and a subsequent, de novo mutation that removes that remaining intact copy of a DNA locus. For an autosomal DNA locus, or for an X-chromosome DNA locus in females, a deletion variant in all copies of the gene means a deletion of the DNA locus on both chromosomes. For a sex-chromosome locus in males, a deletion variant in all copies of the gene means a deletion of the only copy of that locus. For example, in the CYP2A6 gene, there is more than one deletion allele of the same locus present in the population that leads to the complete deletion of the DNA locus.

By “deletion variant antigen” is meant an antigen that is encoded by a gene with a “deletion variant” which, when present, prevents expression of the antigen. Preferably, a deletion variant antigen is not an HLA, MHC antigen, or Rh factor. For example, the antigens encoded by UGT2B17, UGT2B28, TRY6, LCE3C, GSTM1, GSTT1, CYP2A6, PRB1, OR51A2, ORF4F5, GNB1L, MGAM, or MCEE are considered deletion variant antigens because, when the deletion variant is present, expression of the antigen is prevented.

By “deletion variant pattern” is meant a compilation of the determination of the presence or absence of deletion variants present in one or more genes in a biological sample. Deletion variant patterns can be determined at the nucleic acid sequence level or at the antigen expression level using any standard method for nucleic acid sequence determination or antigen expression detection known in the art or described herein. The deletion variant pattern can be determined for one gene, two genes, three or more genes, a genomic locus, a chromosome, or an entire genome for a subject sample. The deletion variant pattern can also be determined for one or more deletion variant antigens. A deletion variant pattern identified for one gene, two genes, three or more genes, a genomic locus, a chromosome, an entire genome, or an antigen for one subject sample can be compared to a deletion variant pattern for the same one gene, two genes, three or more genes, a genomic loci, specified genomic loci, a chromosome, an entire genome, or an antigen identified for a second subject sample. The two patterns are said to be substantially identical if they are more than 50%, 60%, 70%, 80%, 90%, 91%, 92%, 93%, 94%, 95%, 96%, 97%, 98%, 99% or more identical over the one gene, two genes, three or more genes, genomic loci, chromosome, entire genome, or antigen compared. Two subjects with a substantially identical deletion variant pattern are said to be immunocompatible. Deletion variant patterns can be compared over an entire region or only for genes or genomic loci that are relevant to the organ or tissue for which immunocompatibility is desired.

By “deletion variant typing” is meant the process of determining the presence or absence of a deletion variant, preferably a common deletion variant, in a nucleic acid encoding an antigen. Deletion variant typing may or may not be used in combination with HLA typing.

By “deletion variant antigen typing” is meant the process of determining the presence or absence of a deletion variant antigen encoded by a gene having a deletion variant, preferably a common deletion variant. Deletion variant typing may or may not be used in combination with HLA typing.

By “deletion mismatch locus” is meant the absence of a genetic locus from the genome, or subset thereof, of one sample that is not absent (i.e. not homozygous deleted) in the genome, or subset thereof of another sample. Generally, the absence of the genetic locus is due to the presence of a deletion variant in all copies of that locus (i.e., a homozygous deletion).

By “donor” is meant a mammal, preferably a human, from whom an organ or a tissue is removed. The mammal may be alive or dead at the time the organ or tissue is removed. By “potential donor” is meant an individual who is identified as having an organ or tissue suitable for transplant. Generally, a potential donor will be free of disease affecting the organ or tissue to be transplanted. For example, a potential liver donor will generally have a healthy liver and be free of liver cancer, cirrhosis, sepsis, or infection with hepatitis A, B, or C virus or human immunodeficiency virus. A potential bone marrow or peripheral blood donor will generally be free of viral infection, blood cancer, or any type of hematologic disorders. A “preferred donor” is a donor that is matched to a recipient either by standard methods known in the art, such as blood typing, HLA typing, or by the methods described herein, or a combination thereof. Donors can be obtained from a registry of potential donors such as the National Cord Blood Program, United Network for Organ Sharing, National Marrow Donor Program, and any other public or private international, national, state, or local organ procurement organizations or organ donor registries. Information pertaining to potential donors can be entered into a database including name, age, sex, race, blood type, HLA type, and deletion variant typing, deletion variant antigen typing, or deletion variant pattern.

By “donor/recipient match” is meant a donor and a recipient that are identified as having (donor) and needing (recipient) the same organ, tissue, blood, or bone marrow and are immunocompatible. Donor/recipient matches need not be a perfect match but may have sufficiently matched criteria (e.g., blood type, HLA type, antigen type), which can be determined by the skilled artisan or the transplant physician. Preferably, a donor/recipient match will have the same blood type and will be identical for at least 1 deletion variant antigen, preferably 2 or more, 3 or more, 4 or more, 5 or more, and most preferably all of the deletion variant antigens for the biological sample being tested. A donor/recipient match will also preferably have an identical pattern for at least one HLA allele, preferably 2 or more, 3 or more, 4 or more, 5 or more, or all 6 commonly tested HLA alleles (e.g., 2 each for HLA-A, HLA-B, and HLA-DR). Donor/recipient matches can be further screened using additional medical criteria such as size of organ and urgency of need of organ, as well as geographic criteria and other health considerations.

By “expression” is meant the production by cells of a gene or polypeptide detectable by standard art known methods. For example, polypeptide expression is often detected by immunological methods, DNA expression is often detected by Southern blotting or polymerase chain reaction (PCR), and RNA expression is often detected by northern blotting, PCR, or RNAse protection assays.

By “gene” is meant a nucleic acid (e.g., DNA) sequence that comprises coding sequences necessary for the production of a polypeptide, precursor, or RNA (e.g., mRNA, rRNA, tRNA), as well as regulatory sequences that promote or restrict the expression of that gene. The term encompasses the coding region and the sequences located adjacent to the coding region on both the 5′ and 3′ ends. Sequences which regulate the expression of a gene's coding sequence are typically located close (e.g., within a distance of about 10 kb) to the coding sequence and are frequently called “promoter elements.” Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form contains the coding region (“exons”) interrupted with non-coding sequences termed “introns” or “intervening regions” or “intervening sequences.” Introns are segments of a gene that are transcribed into nuclear RNA (hnRNA); introns may contain regulatory elements such as enhancers. Exons are the segments of the DNA that encode the polypeptide. Introns are removed or “spliced out” from the nuclear or primary transcript; introns therefore are absent in the messenger RNA (mRNA) transcript. The mRNA functions during translation to specify the sequence or order of amino acids in a nascent polypeptide. As used herein, the term “nucleic acid” means a polynucleotide such as deoxyribonucleic acid (DNA) or ribonucleic acid (RNA) and encompasses both single-stranded and double-stranded nucleic acid. Total genomic DNA is a particularly useful nucleic acid with which to practice a method of the invention. When detecting a polymorphism in a coding region, mRNA or cDNA are also useful.

By “genome” is meant the complete genetic content of an organism. The genome includes both the genes and the non-coding sequences. By “a subset of the whole genome” is meant a substantial portion of the genome. For example, chromosomal DNA is a preferred subset of the whole genome. In another example, the DNA sequences encoding proteins is a preferred subset of the whole genome. In another example, the DNA sequences encoding proteins that are known to be expressed in a particular organ or tissue type of interest is a preferred subset of the whole genome. In another example, the DNA sequences encoding protein sequences that are known to be presented by the MHC or to elicit antibody responses are a preferred subset of the genome.

By “hematologic disorder” is meant any abnormal condition of any type of blood cell including erythrocytes (red blood cells), platelets, leukocytes, monocytes, granulocytes, lymphocytes. Examples of diseases of the blood include cancers such as Hodgkin's lymphoma, non-Hodgkin's lymphoma, leukemia, multiple myeloma, and myelodysplastic syndrome. Also included are diseases of the immune system, aplastic anemia (when bone marrow stops producing new blood cells), inherited diseases of the bone marrow such as sickle cell anemia, and some metabolic diseases.

By “hybridize” is meant pair to form a double-stranded molecule between complementary polynucleotide sequences, or portions thereof, under various conditions of stringency. (See, e.g., Wahl and Berger (1987) Methods Enzymol. 152:399; Kimmel, Methods Enzymol. 152:507, 1987.) For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “immunocompatibility,” “immunological compatibility,” or “histocompatibility” is meant a condition in which the cells or tissue of one subject do not elicit an immune response by the immune system of another subject. Generally, immunocompatibility is measured by determining the presence of antigens in the cells or tissue of one subject that are absent in the cells or tissue of another subject and would cause the second subject to elicit an immune response against the antigen(s). Examples of such antigens known in the art include the glycosyltransferase enzyme that modifies the carbohydrate content of the red blood cell antigens and determines the blood type of an individual (e.g., Type A, B, AB, or O), HLA antigens, and the Rh antigen. Immunocompatibility can be absolute or relative to another individual based on the number of antigens tested and found in the subjects tested. For example, if a first subject has the same blood type, Rh factor and 3 out of 6 HLA antigens that are identical to one individual and the same blood type, Rh factor antigen, and 5 out of 6 HLA antigens that are identical to the second individual, the first subject is said to be more immunocompatible with the second individual than with the first individual.

By “major histocompatibility complex” or “MHC” is meant a complex of genes encoding cell surface molecules that are required for antigen presentation to T cells. The MHC is a large genomic region or gene family found in most vertebrates containing many genes with important immune system roles. In humans, the MHC is also referred to as the Human Leukocyte Antigen (HLA) and spans almost 4 megabases of chromosome 6. The strongest antigens of the MHC are separated into two classes—class I and class II. Class I and II MHC molecules are found in nearly every cell in the body and are the major determinants used by the body's immune system for recognition and differentiation of self from non-self. The class I MHC molecules are encoded by three loci—HLA A, HLA B, and HLA C—and class II MHC molecules are encoded by three loci—HLA DR, HLA DP, and HLA DQ.

By “polymorphism” is meant the occurrence of different forms, stages, or types in individual organisms or in organisms of the same species, independent of sexual variations, for example, the DNA sequence variations that occur when a single nucleotide (A, T, C, or G) in the genome sequence is altered. One example of a polymorphism is a single nucleotide polymorphism (SNP).

By “predicting the immunocompatibility” is meant determining or identifying the genetic similarities between two individuals or between an individual and a cell, tissue, or organ to be transplanted into that individual.

By “recipient” is meant a mammal, preferably a human, in need of an organ or a tissue transplant. Recipients can also be entered into a registry or a waiting list of subjects in need of an organ or tissue transplant. Information pertaining to recipients that can be entered into a database includes name, age, sex, race, blood type, HLA tissue type, geographic location, and urgency of the needed organ or tissue donation.

By “subject” is meant a mammal, including, but not limited to, a human or non-human mammal, such as a bovine, equine, canine, ovine, or feline.

By “substantially prevents expression” is meant to cause a reduction in the expression of a gene or antigen by at least 50%, 60%, 70%, 80%, 90%, 95%, 99%, or 100% when compared to the expression of the gene or antigen in a sample that does not have a deletion variant in the gene or a deletion variant antigen. The term “substantially prevents expression” also includes a loss or reduction in the expression of a gene or antigen spatially or temporally during development when compared to the expression of the gene or antigen in a sample that does not have a deletion variant in the gene or a deletion variant antigen.

By “tolerize” is meant providing an antigen or nucleic acid sequence encoding an antigen to an individual to reduce or prevent antigen-specific immune responses.

By “transplantation” is meant the transfer of cells, tissues, blood, bone marrow, or organs from one area of the body to another area of the body or from one organism to another. Allogeneic transplantation refers to transplantation between genetically different members of the same species. Nearly all organ and bone marrow transplants are allografts. These may be between brothers and sisters, parents and children, or between donors and recipients who are not related to each other. Autologous transplantation refers to transplantation of an organism's own cell or tissues; autologous transplantation may be used to repair or replace damaged tissue; autologous bone marrow transplantation permits the usage of more severe and toxic cancer therapies by replacing bone marrow damaged by the treatment with marrow that was removed and stored prior to treatment. By xenogenic transplantation is meant transplantation between members of different species; for example, the transplantation of animal organs into humans. Transplantation can refer to the transfer of a healthy organ or tissue such as liver, kidney, heart, pancreas, skin, lungs, and cornea. Transplantation can also refer to the transfer or replacement of blood or bone marrow, for example in as bone marrow transplant (BMT), umbilical cord blood, or peripheral blood stem cell transplant (PBSCT), where diseased blood cells or stem cells can be restored or replaced.

We have discovered a number of common deletion variants in genes that encode for antigens expressed in tissues relevant to immunocompatibility. The conservation of these common deletion variants among multiple individuals, the presence of the antigens encoded by these polymorphic genes in relevant tissues, and the ability of the antigen to elicit an immune response, makes them ideal candidates for screening methods that determine immunocompatibility between two subjects in any situation where compatibility or histocompatibility is desired.

Other features and advantages of the invention will be apparent from the following Detailed Description, the drawings, and the claims.

### BRIEF DESCRIPTION OF THE DRAWINGS

FIG. 1 is a schematic diagram showing the use of SNP genotypes to discover segregating deletion variants. Segregating deletions leave a “footprint” in SNP genotype data by causing physically clustered patterns of null genotypes, apparent Mendelian inconsistencies, and apparent Hardy-Weinberg disequilibrium.

FIGS. 2A-2E show the spatially patterned aberrations in SNP genotypes. FIG. 2A is graph based on pairs of HapMap SNP markers that were typed using different genotyping technologies and showing the more frequent appearance of Mendelian-inconsistent SNP genotypes (“Mendel failures”) at genomic locations close to other Mendel failures when those earlier failures are observed in the same individuals (open circles) but not when they are observed in other individuals (filled circles). FIG. 2B is a graph showing the clustering of population patterns of null genotypes. FIGS. 2C and 2D show the spatially patterned failure of SNP genotype assays at the site of segregating deletions. Tracks show, for each SNP assay (triangles), the pattern of null genotypes across 90 individuals (green track), the pattern of Mendel failure across 60 pairs of relatives (blue track), and the ratio of observed to expected heterozygosity (red track). Physically clustered sets of similarly aberrant genotypes identify a common, 85 kb segregating deletion on chromosome 3q (FIG. 2C) and a common, 10 kb segregating deletion on chromosome 7q (FIG. 2D), both in a sample of 30 trios with European ancestry. FIG. 2E is a graph showing the size distribution of deletion variants identified from regional patterns of aberrant SNP genotypes. A few deletions larger than 100 kb (up to 845 kb) were also observed.

FIGS. 3A-3D show the existence of segregating deletions at the sites of clusters of aberrant genotypes. FIG. 3A is a series of photomicrographs showing fluorescent in situ hybridization (FISH) confirmation of the presence and Mendelian inheritance of an 85-kb deletion at chr4q13.2 at 70.4 MB. FIG. 3B is a graph showing two color-allele-specific fluorescence intensity measurements for a SNP underneath a common deletion on chr4 at 69.5 MB. The measurements show extra genotype clusters (beyond the 2-3 clusters typically observed for SNPs), corresponding to individuals who were subsequently determined to carry hemizygous and homozygous deletions of the locus. FIG. 3C is a series of photographs of gels showing confirmation by PCR of a predicted population pattern of homozygous deletion of sequence on chr8p23.3 at 2.4 MB. Yellow arrows indicate the individuals predicted (from having multiple null genotypes at the locus) to carry homozygous deletions. FIG. 3D is a graph showing measurements of copy number obtained by quantitative PCR (shown here for a deletion on chr4 at 70.5 MB) fall into three discrete clusters, allowing accurate inference of the deletion genotype in each individual.

FIG. 4 is a series of graphs showing inter-individual variation in gene expression due to gene copy number variation. Each graph shows the measure expression level of each gene (Monks et al., Am. J Hum. Genet. 75:1094-1105(2004)) in lymphoblastoid cell lines from individuals who were determined by quantitative PCR to have 0, 1, and 2 gene copies.

FIGS. 5A-5C show linkage disequilibrium of deletion variants with SNPs. FIG. 5A is a series of graphs showing linkage disequilibrium (r²) of gene deletion polymorphisms with SNPs. For each gene deletion, strong linkage disequilibrium is observed with SNPs to the left and right of the deletion breakpoints (red dotted lines). FIG. 5B is an image generated using the Bifurcator program (Fry in “Computational Information Design,” Doctoral thesis, MIT, Cambridge Mass., 2005) showing the residence of the UGT2B28 deletion allele on the same core haplotype in European (CEU) and Yoruba (YRI) populations. Letters indicate the consensus haplotype in each population. FIG. 5C is a graph showing haplotype homozygosity across flanking SNPs in individuals homozygous for 51 experimentally validated deletions (red); in randomly selected control individuals at the same deletion loci (black); in individuals homozygous for a frequency- and population-matched set of SNP variants (blue); and in randomly selected control individuals at these SNP loci (yellow).

FIGS. 6A-6D show physical clustering of patterns of apparent Mendelian inconsistency and null genotypes in the HapMap data. FIG. 6A shows “Mendel failure profiles.” Binary patterns of apparent Mendelian inconsistency across the 60 relative-pairs in a population, are more likely to be observed in the proximity of similar profiles at nearby SNPs. FIG. 6B shows “null genotype profiles.” Binary patterns of null genotypes across the 90 individuals in a population, are more likely to be observed in the proximity of similar profiles at nearby SNPs. FIG. 6C shows clustering p-values for Mendel failure profiles that show a generally uniform distribution with an excess of extremely low p-values from which candidate deletion variants were identified. FIG. 6D shows clustering p-values for null genotype profiles that show a generally uniform distribution, with an excess of extremely low p-values from which candidate variants were identified.

FIGS. 7A-7F show the linkage disequilibrium (r2) between gene deletion polymorphisms and nearby SNPs in three population samples. The predicted locations of the deletion breakpoints are shown by dotted lines. FIG. 7A shows the linkage disequilibrium of TRY6. FIG. 7B shows the linkage disequilibrium of LCE3C. FIG. 7C shows the linkage disequilibrium of UGT2B28. FIG. 7D shows the linkage disequilibrium of UGT2B17. FIG. 7E shows the linkage disequilibrium of GSTM1. FIG. 7F shows the linkage disequilibrium of GSTT1.

### DETAILED DESCRIPTION

The genetic sequences of different people are remarkably similar. When the chromosomes of two humans are compared, their DNA sequences can be identical for hundreds of bases. But at about one in every 1,200 bases, on average, the sequences will differ. Differences in individual bases are by far the most common type of genetic variation. These genetic differences are known as single nucleotide polymorphisms, or SNPs. The International HapMap Project is focused on identifying the basis for a large fraction of the genetic diversity in the human species by identifying most of the approximately 10 million SNPs estimated to occur commonly in the human genome.

For geneticists, SNPs act as markers to locate genes in DNA sequences. However, testing all of the 10 million common SNPs in a person's chromosomes would be extremely expensive. The development of the HapMap is a global collaboration designed to enable geneticists to take advantage of how SNPs and other genetic variants are organized on chromosomes. Genetic variants that are near each other tend to be inherited together. For example, all of the people who have an adenine rather than a guanine at a particular location in a chromosome can have identical genetic variants at other SNPs in the chromosomal region surrounding the adenine. These regions of linked variants are known as haplotypes.

In many parts of the human chromosomes, just a handful of haplotypes are found. For example, in a given population, 55% of people may have one version of a haplotype, 30% may have another, 8% may have a third, and the rest may have a variety of less common haplotypes. The International HapMap Project is identifying these common haplotypes in four populations from different parts of the world.

One type of human genetic variation consists of deletion variants—segments of the human genome that are present in some individuals and absent in others. The locations of common deletion variants in the human genome are largely unknown, as is the best way to determine the association of such variants with disease. To address these questions, we developed an approach for using the HapMap to discover, localize, and analyze common deletion variants. We found hundreds of deletion variants, 1 kb-745 kb in size, including common deletion variants that were observed as homozygous deletions in a number of expressed genes that are specifically expressed in organs relevant to transplantation, such as liver, prostate, kidney, intestine, and skin. These common deletion variants prevent the expression of the protein, or antigen, encoded by these genes.

The present invention features methods for identifying immunocompatible subjects by determining the presence or absence of deletion variants, preferably a deletion variant in all copies of the gene, that substantially prevents expression of either the gene or the antigen encoded by a gene. The lack of expression of the gene or of an antigen encoded by the gene, respectively, is used to identify subjects or subject samples that are immunocompatible. Screening subjects for immunocompatibility is used, for example, to identify donor/recipient matches for transplantation, to identify maternal/fetal compatibility issues in prospective parents, and to identify bone marrow donors that are not immunocompatible with a recipient and can be used to provoke an immune response to the tumor cells in a recipient having a blood cancer. Therefore, the present invention provides methods for immunocompatibility typing which can be used alone or together with previously known typing techniques to manage, measure, prevent, and provoke histoincompatibility.

Identification of Common Deletion Variants

We used data from the International HapMap Project, including about 1.3 million SNP assays in 270 individuals of European, Yoruban, and Chinese and Japanese ancestry, to identify clusters of regionally aberrant genotype patterns (see Examples below). We validated the presence of polymorphic deletions by fluorescence in situ hybridization (FISH), fluorescence allelic-intensity measurements, and PCR. Altogether, more than 80 common deletions were validated by one or more of these approaches

The deletion alleles were linked to the same SNP alleles in each population, suggesting that each deletion derived from an ancestral mutation that occurred before humans migrated from Africa to Europe and Asia. The observed levels of linkage disequilibrium indicates that these common deletion variants are highly conserved among individuals and that SNPs can be used to discover, analyze, and serve as markers for these variants.

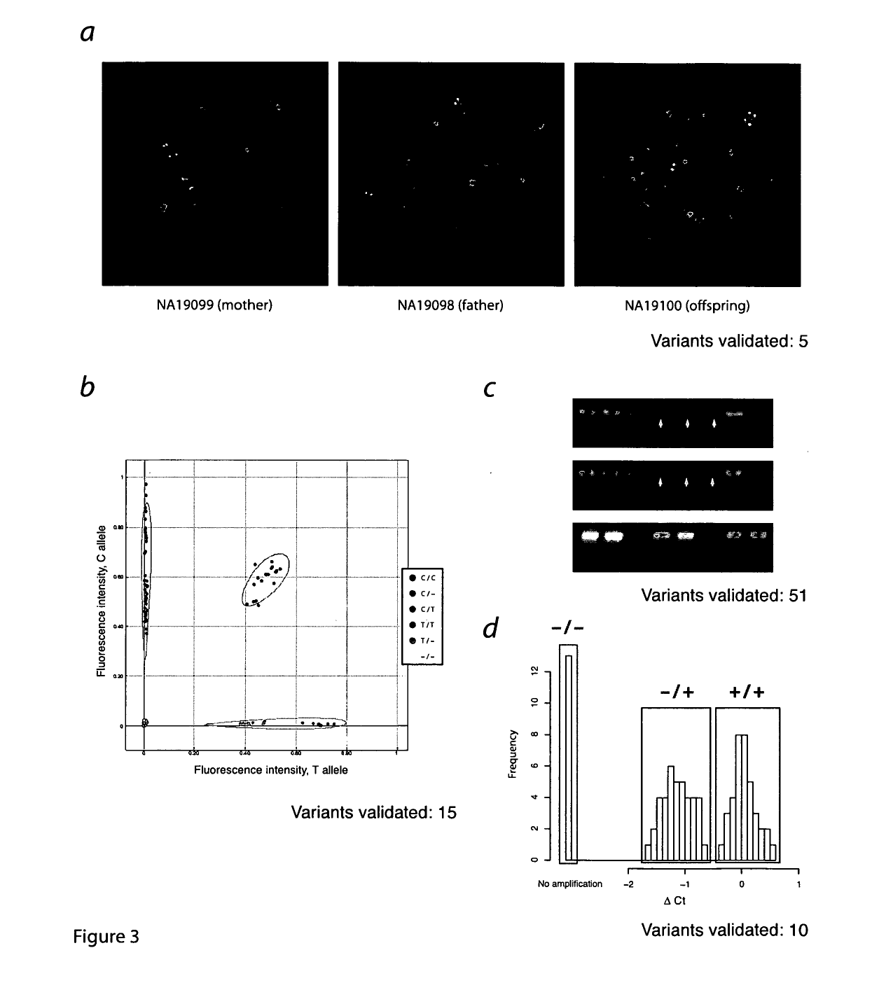

Thirteen protein-coding genes were disrupted or entirely removed by common deletions (Table 1). These common deletion variants were found in multiple genes with roles in drug response, olfaction, and sex steroid metabolism. To learn more about these common gene deletion variants, we developed quantitative PCR assays for distinguishing individuals with 0, 1, and 2 gene copies (FIG. 3A), and used these assays to type seven gene deletion variants in all the HapMap individuals. The resulting genotypes showed Mendelian inheritance, Hardy-Weinberg equilibrium, and stable transmission rates, suggesting that they behave as stable, segregating, germline genetic variants. The deletion variants were observed in individuals of European, Yoruban, and Chinese and Japanese ancestry, though the frequency of each deletion haplotype varied from population to population (Table 1).

TABLE 1
Common Deletion Variants.

| | | | | Population frequency of | | | Linkage disequilibrium | | |
|---|---|---|---|---|---|---|---|---|---|
| | | | | deletion variant | | | (tagging SNP R²) | | |
| | | | Exons | | Chinese/ | | | Chinese/ | |
| Gene | Function | Expression | deleted | European | Japanese | Yoruba | European | Japanese | Yoruba |
| UGT2B17 | Sex steroid hormone | liver, prostate | all | 30% | 84% | 22% | 1.00 | 0.96 | 0.63 |
| | metabolism | | | | | | | | |
| UGT2B28 | Sex steroid hormone | liver, kidney | all | 13% | 15% | 35% | 1.00 | 1.00 | 0.90 |
| | metabolism | | | | | | | | |
| TRY6 | Proteolysis | not known | all | 41% | 74% | 12% | 1.00 | 1.00 | 1.00 |
| LCE3C | Epidermal cornified | internal | all | 56% | 69% | 30% | 0.93 | 1.00 | 0.92 |
| | envelope | epithelia | | | | | | | |
| GSTM1 | Detoxification, drug | liver | all | 76% | 70% | 48% | 0.66 | 0.95 | 0.87 |
| | metabolism | | | | | | | | |
| GSTT1 | Detoxification, drug | glands, | all | 38% | 63% | 62% | 0.85 | 0.61 | 0.38 |
| | metabolism | kidney | | | | | | | |
| CYP2A6 | Detoxification, drug | liver | all | 25% | 55% | 18% | 0.22 | 0.47 | 0.09 |
| | metabolism | | | | | | | | |
| PRB1 | Secreted salivary | salivary | #1-2 of 4 | N.D. | N.D. | N.D. | N.D. | N.D. | N.D. |
| | proteoglycan | gland, trachea | | | | | | | |
| OR51A2 | Olfactory receptor | olfactory | all | 50% | 19% | 28% | 0.40 | 0.51 | 0.20 |
| | | epithelium | | | | | | | |
| OR4F5 | Olfactory receptor | olfactory | all | N.D. | N.D. | N.D. | N.D. | N.D. | N.D. |
| | | epithelium | | | | | | | |
| GNB1L | Guanine nucleotide | heart | all | N.D. | N.D. | N.D. | N.D. | N.D. | N.D. |
| | binding | | | | | | | | |
| MCEE | Methylmalonyl CoA | various | #3 of 3 | N.D. | N.D. | N.D. | N.D. | N.D. | N.D. |
| | epimerase | | | | | | | | |
| MGAM | Maltase glucoamylase | N.D. | N.D. | N.D. | N.D. | N.D. | N.D. | N.D. | N.D. |

N.D. = no data

These common deletion variants were detected in several tissues including liver, prostate, kidney, heart, and skin, all of which are important to immunocompatibility and transplantation. The expression products from these genes, also known as antigens, are absent in an individual having the common deletion variant. As a result, the immune system of that individual would not be exposed to the antigen, and, if exposed, would recognize the antigen as foreign and respond by mounting an immune response to the antigen. The conservation of these common deletion variants, particularly among people of a shared ancestry, and the ability of the encoded antigens to elicit an immune response indicates that these common deletion variants and the encoded antigens are an effective tool for screening individuals for immunocompatibility, particularly with respect to the organs or tissues in which the antigen is expressed.

Methods for the use of common deletion variants, for example, those identified in Table 1, to screen for and manage immunocompatibility are described in detail below. It should be understood by the skilled artisan that any deletion variant, particularly a common deletion variant, that affects the expression of any antigen can be used in the methods described herein to screen for and manage immunocompatibility or to provoke histoincompatibility, if desired. In addition, it should be understood that any methods for sequencing all or part of a subject's genome, or determining the deletion variant pattern or deletion variant antigen pattern for a subject can be used to identify additional deletion variants in expressed antigens and to screen for and manage immunocompatibility.

Methods for Deletion Variant Typing

Individual subjects can be typed for the presence or absence of common deletion variants in a biological sample using methods for detection of the deletion in the gene or methods for detection of the antigen encoded by the gene. The biological sample used to detect the gene or protein can be any biological material from the subject (e.g., the graft recipient, potential donor, mother, fetus, father, or prospective parent) that contains the antigen or nucleic acids encoding the antigen. For detection of deletion variant antigens, the biological sample is preferably a sample in which the antigen is normally expressed. Desirably the biological material is a bodily fluid, such as blood, serum, plasma, amniotic fluid, cerebrospinal fluid, saliva, urine, and semen, or a cell or tissue in which the antigen or nucleic acid encoding the antigen is expressed. In the case of an organ transplant, the biological sample is desirably a biopsy of the organ to be transplanted and the antigen or nucleic acid encoding the antigen is expressed in the organ. In the case of a bone marrow or peripheral blood transplant, the biological sample is preferably blood, serum, or plasma in which the antigen or nucleic acid encoding the antigen is expressed.

Methods of detecting deletion variants in a nucleic acid are well known to those skilled in the art. In one example, polymerase chain reaction (PCR) can be used to detect a deletion variant in a nucleic acid. Oligonucleotide PCR primers that flank a known deletion polymorphism can be used to amplify genomic DNA spanning the deletion breakpoints in individuals carrying the deletion allele; alternatively, oligonucleotide primers inside the deleted sequence can be used to amplify genomic DNA selectively in individuals carrying the other (non-deletion) allele. The amplified genomic DNA can then be sequenced, analyzed by fluorescence quantitation, resolved on a gel, or otherwise analyzed, and the presence or absence of a deletion variant can be determined. These PCR-based methods can be combined to identify individuals carrying 0, 1, or 2 copies of the deletion allele. Furthermore, quantitative PCR can be used to compare the abundance of a polymorphically deleted locus to the abundance of a control locus, and thereby infer copy number, and thereby infer the deletion status of an individual. Methods for PCR amplifying and sequencing a nucleic acid molecule are well known to those skilled in the art (see, for example, Sambrook et al., Molecular Cloning: A Laboratory Manual, 2nd ed., Cold Spring Harbor Press, Plainview, N.Y. (1989); Ausubel et al., Current Protocols in Molecular Biology (Supplement 47), John Wiley & Sons, New York (1999); Dieffenbach and Dveksler, PCR Primer: A Laboratory Manual, Cold Spring Harbor Press (1995)). The following are examples of PCR primers and quantitative fluorescent probes which we have used to successfully genotype deletion polymorphisms in DNA samples from individuals:

PMP22 (control)

| | | |
|---|---|---|
| primer1 | CCCTTCTCAGCGGTGTCATC | (SEQ ID NO: 1) |
| primer2 | ACAGACCGTCTGGGCGC | (SEQ ID NO: 2) |
| probe | VIC-TTCGCGTTTCCGCAAGAT- | (SEQ ID NO: 3) |
| | MGBNFQ | |

Note:
VIC is the fluorescent label commonly known as “VIC” (available, for example, from Applied Biosystems) and MGBNFQ is a non-fluorescent quencher molecule (available, for example, from Applied Biosystems).

| | | |
|---|---|---|
| GSTM1 | | |
| primer1 | CTGTGTCCACCTGCATTCG | (SEQ ID NO: 4) |
| primer2 | GAGACCGGGCACTCACTGT | (SEQ ID NO: 5) |
| probe | 6FAM-TCAGTCCTGCCATGAGCAGGC- | (SEQ ID NO: 6) |
| | BHQ1 | |

Note:
6FAM is the fluorescent label commonly known as “6FAM” (available, for example, from IDT) and BHQ-1 is a non-fluorescent quencher molecule (available, for example, from IDT).

| | | |
|---|---|---|
| GSTT1 | | |
| primer1 | GGGATGGAAAGTCACGTCCT | (SEQ ID NO: 7) |
| primer2 | AGAGACTGGGACAGCGTCAA | (SEQ ID NO: 8) |
| probe | 6FAM-CAGAATCTCAGCAGCTGGGCC | (SEQ ID NO: 9) |
| | A-BHQ1 | |
| CYP2A6 | | |
| primer1 | AGGATGGGGACTTTTCCTTT | (SEQ ID NO: 10) |
| primer2 | TCCTCATCTTCAGCTGTTGG | (SEQ ID NO: 11) |
| probe | 6FAM-CATTCAGGATTCTGGGCTTGC | (SEQ ID NO: 12) |
| | TCC-BHQ1 | |
| OR51A2 | | |
| primer1 | TGCCAATTGCCTACTGTTTG | (SEQ ID NO: 13) |
| primer2 | AGCAACAGTGGAAGGAGAGAA | (SEQ ID NO: 14) |
| probe | 6FAM-GACAACATAACCAAGTGGGGC | (SEQ ID NO: 15) |
| | TTATTTTC-BHQ1 | |
| PRB1 | | |
| primer1 | TGAAGGGACCTCAGTAGTTGG | (SEQ ID NO: 16) |
| primer2 | TGACAGGCATGGTTCTTCTG | (SEQ ID NO: 17) |
| probe | 6FAM-CTGACTTTCTAGCAAGG- | (SEQ ID NO: 18) |
| | MGBNFQ | |
| UGT2B17 | Applied Biosystems | |
| | Hs00854486_sH | |
| UGT2B28 | Applied Biosystems | |
| | Hs00852540_s1 | |
| LCE3C | Applied Biosystems | |
| | Hs00708773_s1 | |

Sequence analysis, which is any manual or automated process by which the order of nucleotides in a nucleic acid is determined, also can be useful for determining the presence or absence of a common deletion variant. It is understood that the term sequence analysis encompasses chemical (Maxam-Gilbert) and dideoxy enzymatic (Sanger) sequencing as well as variations thereof. Thus, the term sequence analysis includes capillary array DNA sequencing, which relies on capillary electrophoresis and laser-induced fluorescence detection and can be performed using, for example, the MegaBACE 1000 or ABI 3700. Also encompassed by the term sequence analysis are thermal cycle sequencing (Sears et al., Biotechniques 13:626-633 (1992)); solid-phase sequencing (Zimmerman et al., Methods Mol. Cell Biol. 3:39-42 (1992)) and sequencing with mass spectrometry such as matrix-assisted laser desorption/ionization time-of-flight mass spectrometry MALDI-TOF MS (Fu et al., Nature Biotech. 16: 381-384 (1998)). Sequence analysis can be used to determine the sequence of a particular genetic loci known to have a common deletion variant, an entire gene known to contain a common deletion variant, a chromosome, or the entire genome of a subject. The term sequence analysis also includes, for example, sequencing by hybridization (SBH), which relies on an array of all possible short oligonucleotides to identify a segment of sequences present in an unknown DNA (Chee et al., Science 274:61-614 (1996); Drmanac et al., Science 260:1649-1652 (1993); Drmanac et al., Nature Biotech. 16:54-58 (1998), Margulies et al., Nature 437:376-380 (2005) and Bentley, Curr. Opin. Genet. Dev. 16:545-552 (2006)). The whole genome approach to typing individual subjects for the presence or absence of common deletion variants is described in detail below.

Other methods for detecting the presence or absence of a deletion variant include electrophoretic analysis and restriction fragment length polymorphism (RFLP) analysis. Electrophoretic analysis, as used herein in reference to one or more nucleic acid molecules such as amplified fragments, means a process whereby charged molecules are moved through a stationary medium under the influence of an electric field. Electrophoretic migration separates nucleic acid molecules primarily on the basis of their charge, which is in proportion to their size. The term electrophoretic analysis includes analysis using both slab gel electrophoresis, such as agarose or polyacrylamide gel electrophoresis, and capillary electrophoresis. Capillary electrophoretic analysis is generally performed inside a small-diameter (50-100-μm) quartz capillary in the presence of high (kilovolt-level) separating voltages with separation times of a few minutes. Using capillary electrophoretic analysis, nucleic acids are conveniently detected by UV absorption or fluorescent labeling, and single-base resolution can be obtained on fragments up to several hundred base pairs. Such methods of electrophoretic analysis, and variants thereof, are well known in the art, as described, for example, in Ausubel et al., Current Protocols in Molecular Biology, John Wiley & Sons, Inc. New York (1999).

Restriction fragment length polymorphism (RFLP) analysis also can be useful for determining the presence or absence of a deletion variant (Jarcho et al., in Current Protocols in Human Genetics, Dracopoli et al., eds., pages 2.7.1-2.7.5, John Wiley & Sons, New York (1994); Innis et al., (Ed.), PCR Protocols, San Diego: Academic Press, Inc. (1990)). As used herein, restriction fragment length polymorphism analysis means any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site. For example, if the restriction enzyme recognizes a specific base sequence that is present in the nucleic acid sequence containing the deletion variant, then a subject having the deletion variant would not have cleavage at that restriction enzyme site and would therefore produce a different enzymatic cleavage pattern than a subject lacking the deletion variant and having the restriction enzyme site.

Other methods for detecting the presence or absence of a deletion variant at a polymorphic site include allele-specific oligonucleotide (ASO) hybridization. Allele-specific oligonucleotide hybridization is based on the use of a labeled oligonucleotide probe having a sequence perfectly complementary, for example, to a known or predicted deletion variant site. A heteroduplex mobility assay (HMA) is another well-known assay that can be used to detect a common deletion variant according to a method of the invention. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational polymorphism (SSCP) can also be used to detect the presence or absence of a deletion variant (see Hayashi, PCR Methods Applic. 1:34-38 (1991)). This technique can be used to detect deletions based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

SNP genotyping can also be used to detect the presence or absence of a deletion variant. We have observed that common deletion polymorphisms are generally in linkage disequilibrium with nearby SNPs, which suggests that specific SNP genotyping assays could be used to indirectly detect a deletion polymorphism. In this technique, a SNP that is known to be in linkage disequilibrium with a deletion polymorphism, such that individuals carrying the deletion almost always carry a particular variant of the SNP, is used as a marker for the presence of the deletion. Individuals can be typed for the SNP as a way of indirectly typing for the deletion. Techniques for deriving SNP genotypes include hybridization to allele-specific complementary sequences on microarrays or beads, as well as allele-specific primer extension.

We have further observed that genotyping of a SNP that is inside a deleted region can also be used to infer the presence of a deletion that removes the site of the SNP. In particular, the presence of the deletion causes particular SNP genotyping results, including null genotypes, apparent mendelian inconsistencies, and reductions in intensity measurements. Techniques for deriving SNP genotypes include hybridization to allele-specific complementary sequences on microarrays or beads, as well as allele-specific primer extension.

Denaturing gradient gel electrophoresis (DGGE) also can be used to detect a deletion variant. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

In addition to using DGGE as described above, other methods to detect heteroduplexes include temperature gradient gel electrophoresis (TGGE), constant denaturant gel electrophoresis (CDGE), and base excision sequence scanning (BESS) (Gupta, The Scientist 13:25-28 (1999)). Other methods include oligonucleotide ligation assay (OLA) in which a PCR-amplified target is hybridized to two oligonucleotides, one tagged, for example, with biotin, and the other with a reporter molecule and then ligated with DNA ligase. If the tag and reporter oligonucleotides are ligated, the tagged molecule can be used to isolate the ligated oligonucleotide and the reporter molecule can be detected.

Other well-known approaches for determining the presence or absence of a deletion variant include automated sequencing and RNAase mismatch techniques (Winter et al., Proc. Natl. Acad. Sci. 82:7575-7579 (1985)). In view of the above, one skilled in the art realizes that the methods of the invention for determining the presence or absence of a deletion variant in an individual can be practiced using any one of the well known assays described above, or another art-recognized assay for genotyping. Furthermore, one skilled in the art understands that individual alleles can be detected by any combination of molecular methods (see, in general, Birren et al. (Eds.) Genome Analysis: A Laboratory Manual Volume 1 (Analyzing DNA) New York, Cold Spring Harbor Laboratory Press (1997)).

Additional methods for determining the presence of deletion variants include fluorescence in situ hybridization (FISH) and fluorescence allelic-intensity measurements, examples of which are described in the Examples below. FISH is used to visualize the presence or absence of DNA sequence on chromosomes, via hybridization of a fluorescent probe to the chromosome in site.

In addition to the above methods for detecting the presence of a known human deletion polymorphism, additional methods, known to those versed in the art, can be used to scan the genome of one individual for deletions of DNA sequences which are present in other individuals. One such method is microarray hybridization, in which DNA from a subject is probed with a microarray of nucleic acids containing human genomic sequences, and the user identifies microarray probes which are not bound by that individual's genomic DNA. Another such method is whole-genome sequencing, in which the DNA from an individual is systematically sequenced. In this application, the practitioner could look for nucleic acid sequences which appear to be absent from that individual's sequence but which are known to be present in other individuals. Another such method is subtractive hybridization, in which two DNA samples are compared by molecular techniques which allow DNA sequences that are present in the first sample to be selectively removed from the second sample, leaving only those DNA sequences that are present in the second sample and not in the first sample. Such an approach could be used to identify genomic loci that were deleted in the individual from whom the first sample was obtained but present in the second individual from which the second sample was obtained.

Methods for detecting the presence or absence of a deletion variant antigen are also well known in the art and include, for example, immunoassays to detect the presence of an antigen in the biological sample of the subject. Polyclonal or monoclonal antibodies specific for each antigen can be used in any standard immunoassay format (e.g., ELISA, sandwich ELISA, Western blot, or RIA; see, e.g., Ausubel et al., supra) to determine the presence of the antigen. Standard methods for enzyme immunoassays can also be used to detect antigens that are present on enzymes, such as GSTM1, GSTT1, UGT2B17, UGT2B28, and CYP2A6. ELISA assays are the preferred method for measuring levels of any one or more of the following antigens: UGT2B17, UGT2B28, TRY6, LCE3C, GSTM1, GSTT1, CYP2A6, PRB1, OR51A2, ORF4F5, GNB1L, MGAM, and MCEE. Particularly preferred, for ease and simplicity of detection, and its quantitative nature, is the sandwich or double antibody ELISA of which a number of variations exist, all of which are contemplated by the present invention. For example, in a typical sandwich ELISA, unlabeled antibody that recognizes the antigen is immobilized on a solid phase, e.g. microtiter plate, and the sample to be tested is added. After a certain period of incubation to allow formation of an antibody-antigen complex, a second antibody, labeled with a reporter molecule capable of inducing a detectable signal, is added and incubation is continued to allow sufficient time for binding with the antigen at a different site, resulting with a formation of a complex of antibody-antigen-labeled antibody. The presence of the antigen is determined by observation of a signal, which may be quantitated by comparison with control samples containing known amounts of antigen.

Immunohistochemical techniques can also be utilized for detection of any of the antigens in a tissue biopsy sample. For example, a tissue sample can be obtained from a subject, sectioned, and stained for the presence of the antigen using an antibody that specifically binds the antigen and any standard detection system (e.g., one that includes a secondary antibody conjugated to an enzyme, such as horseradish peroxidase). General guidance regarding such techniques can be found in, e.g., Bancroft et al., Theory and Practice of Histological Techniques, Churchill Livingstone, 1982 and Ausubel et al., supra).

The methods described herein can be used to detect one or more deletion variants, preferably common deletion variants, in a single gene or in more than one gene. For example, an individual can be typed for the presence of one, two, three, four, five, six or more common deletion variants in nucleic acids encoding one, two, three, four, five, six or more different antigens (e.g., UGT2B17, UGT2B28, TRY6, LCE3C, GSTM1, GSTT1, CYP2A6, PRB1, OR51A2, ORF4F5, GNB1L, MGAM, and MCEE). The methods described herein can be used to detect one or more deletion variant antigens. For example, an individual can be typed for the presence or absence of one, two, three, four, five, six or more deletion variant antigens. While it is preferred that two subjects are a perfect match for each and every deletion variant or deletion variant antigen tested, individuals can be ranked for immunocompatibility depending on the number of matches and the relative importance of the antigen. For example, an individual in need of a liver transplant would seek a donor having a common deletion variant type match at the UGT2B17, UGT2B28, and GSTM1 loci, all of which are expressed in the liver, but may not be matched for common deletion variants at the OR51A2 loci, which is expressed in the olfactory epithelium. Two subjects can also be typed for deletion variant patters or deletion variant antigen patterns in which one or more genes, genomic loci, chromosome, or entire genome is assayed using the methods described herein to determine the presence or absence of deletion variants throughout the one or more genes, genomic loci, chromosome, or entire genome assayed. The information is then compiled into a deletion variant pattern for each subject and can be compared either for overall substantially identical patterns or for substantial identity within a defined set of genes or antigens, e.g., those expressed in an organ or tissue being transplanted. For example, a subject in need of a liver transplant may show deletion variants in 3 genes expressed in the kidney and 1 gene expressed in the liver and a potential donor has a deletion variant in 1 of the same genes expressed in the kidney and the same 1 gene expressed in the liver. The potential liver donor is identified as immunocompatible because of the 100% identity of the deletion variant pattern in the relevant tissue (i.e., the liver).

Methods for Whole Genome Sequence Analysis to Determine Immunocompatibility

As described above, sequence analysis, including any manual or automated process, can be used for determining the presence or absence of a common deletion variant. Such sequence analysis can also be used to analyze the genome, or a subset thereof, of an individual subject and to compare that subject's genome sequence, or subset thereof, to the genome sequence or the same subset thereof, in a second individual or a cell, tissue, or organ from the second individual. This type of whole genome, or subset thereof, sequence analysis can be used to search for or identify a deletion variant that is present in one individual and absent in a second individual. The deletion variant can vary in size from 100, 200, 300, 400, 500, 600, 700, 800, 900, or 1000 bp, or 2 kb, 3 kb, 4 kb, 5 kb, 7 kb, 8 kb, 9 kb, 10 kb, 100 kb, 200 kb, 300 kb, 400 kb, 500 kb, 600 kb, 700 kb, 745 kb, 800 kb, 900 kb, or 1000 kb in length. A deletion variant present at a particular loci in one individual and absent in a second individual is called a deletion mismatch loci.

The identification of a deletion mismatch loci between two individuals is predictive of histoincompatibility if:

(i) there is a homozygous deletion variant in a loci in the genome of a first subject (e.g., a candidate bone marrow donor) but not in a loci in the genome of the second subject (e.g., a candidate bone marrow recipient, (ii) there is a homozygous deletion in a loci in the genome of a first subject (e.g., a candidate organ recipient) but not in a loci in the genome of the second subject (e.g., a candidate organ donor), or (iii) there is a homozygous deletion in a loci in the genome of a first subject (e.g., a candidate mother) but not in a loci in the genome of the second subject (e.g., the candidate father, embryo, fetus, or miscarriage).

Alternatively or additionally, the sequence of the genome or subset thereof of the first subject can be compared to a reference genome DNA sequence, where the reference genome sequence can be the DNA sequence from a third subject or from a composite of multiple subjects. The identification of a deletion mismatch loci between the first subject and the reference genome DNA sequence is then carried out as described above and used to predict histoincompatibility as described above.

The whole-genome analysis can be performed using any sequencing technique known in the art or described herein. In one example, a whole genome sequencing approach can be used where millions of genome-wide sequence reads are obtained from the patient's DNA. Technologies available for massively parallel sequencing include sequencing by synthesis in arrays such as on fiber optic slides and single-molecule sequencing via nanopores (Margulies et al., Nature 437:376-380 (2005) and Bentley, Curr. Opin. Genet. Dev. 16:545-552 (2006)). Homozygous deletions are identified as loci which are not covered by any sequence reads, despite overall sequencing having been performed at a sufficient depth to have covered all genomic loci present in that individual.

Another technique useful for the whole genome sequence analysis is genomic hybridization. For this method, patient DNA is labeled with a suitable marker (typically a fluorescent molecule) with or without amplification, and hybridized to an array consisting of DNA probes. These probes can consist of oligonucleotides, plasmids, fosmids, or other genomic clones. Deletions are identified from probes for which the patient's DNA fails to yield the appreciable hybridization signal that is normally observed in DNA from other individuals or fails to yield hybridization signal beyond that would be expected from cross-hybridization to other genomic sequences.

Additional techniques for whole genome sequence analysis are described in Bentley, supra, (herein incorporated by reference in its entirety) and include microelectrophoresis and single molecule sequencing.

Immunocompatibility between two subjects can be determined by the identification of deletion mismatch loci, where two subjects would be considered not immunocompatible if there is at least one, two, three, four, five, six, seven, eight, nine, ten or more homozygous deletion mismatch loci identified between the two subjects; or when a scoring system, which combines information across multiple deletion mismatch loci, is determined to have an appropriately high mismatch score. Preferably, the one or more deletion mismatch loci would remove the protein-coding sequences and prevent expression of the encoded antigen in the individual homozygous for the deletion. Alternatively or additionally, a scoring system can be used to determine the relevance of each deletion mismatch locus identified between the two subjects. The scoring system would score each of the homozygous deletion mismatch loci for its potential contribution to antigenicity, and produce a composite score which combines information across all deletion mismatch loci, and potentially combines this with additional information relevant to histocompatibility, such as the subjects' sex and the subjects' HLA types. For example, a scoring system could assign points for deletions which remove protein-coding sequences for which the encoded proteins are generally expressed in tissues relevant to the immune response considered in the clinical application. For example, for kidney transplant, deletion variants in genes encoding proteins which are expressed in the kidney are assigned points. Additional points are awarded if those deletions affect protein-coding sequences which (i) encode peptide sequences known or predicted to be presented by that individual's HLA alleles or (ii) contain sequences which are particularly accessible to antibodies, such as sequences encoding extracellular domains of proteins.

In the scoring system described above, donor-recipient pairs with a high “deletion mismatch score” are interpreted to be more likely to have histoincompatibilities; such a diagnosis might recommend the use of a different donor, or the application of a tolerization regimen, or the further investigation of any particular deletion mismatches identified by this analysis. This further investigation could include testing the relevant donor or patient for pre-existing antibodies or pre-existing T-cell responses to the antigen encoded by the genomic region(s) identified as the deletion loci.

Statistical analysis or metrics for prioritization or comparison of genomic information are known in the art and can be applied to the methods herein to prioritize and compare the deletion mismatch loci between two subjects and to generate a composite mismatch score reflecting mismatches (including deletion mismatches) at multiple loci. Examples of such analytical methods include naïve Bayesian scoring, decision trees, and boosting; these and similar approaches are routinely applied to genome-scale data sets to derive focused predictions (Jansen et al., Science 302: 449-453, 2003; Calvo et al., Nat Genet 38: 576-582, 2006).

Uses of the Deletion Variants to Measure and Manage Immunocompatibility

We have discovered a number of common deletion variants, particularly among people of a shared ancestry, in genes that encode for antigens expressed in tissues relevant to immunocompatibility. The conservation of these common deletion variants among multiple individuals, the presence of the antigens encoded by these polymorphic genes in relevant tissues, and the ability of the antigen to elicit an immune response, makes them ideal candidates for screening methods that determine immunocompatibility in any situation where immunocompatibility or lack of immunocompatibility is desired.

For example, the methods described herein can be used to detect common deletion variants to determine immunocompatibility between a subject in need of a transplant (a recipient) and a potential donor. These methods can also be used to screen for maternal/fetal incompatibility in cases of spontaneous abortion or among prospective parents having difficulty conceiving. The methods for identifying common deletion variants can also be used to identify a bone marrow donor for a recipient having a blood cancer where the recipient and the donor are not immunocompatible. In this case, a donor's immune system would attack the cancer cells that remain in the recipients blood system thereby enabling the transplanted bone marrow to not only replace the host's bone marrow but also to aid in the treatment of the cancer by killing off any remaining cancer cells present in the recipient. All of these uses are described in detail below.

Organ, Bone Marrow, and Blood Transplantation

Despite the increased success of organ and bone marrow transplantation in recent decades, the overall success is limited by the likelihood of graft rejection and the potentially fatal effects of GVHD or HVGD. In GVHD, most commonly seen in bone marrow transplants, the immune cells in the donor's graft recognize the antigens in the recipient as foreign and mount an immune attack against the host cells. In HVGD, most commonly seen in organ transplants, the recipient's immune system recognizes the antigens expressed in the donor organ graft as foreign and mounts an immune attack against the graft. Although in some cases the immune response can be treated using immunosuppressive drugs, the problems that arise from these drugs presents additional health related complications.

Blood typing and tissue typing for HLA antigens are the most common screens used today for determining immunocompatibility between a recipient and a potential donor prior to transplantation. However, these methods, when used alone, are not always effective or sufficient due to the inadequacies of HLA typing methods and the presence of additional antigens that can elicit an immune response

The deletion variants identified using the methods described herein are useful for screening individuals for immunocompatibility prior to transplantation. In general, a biological sample is obtained from the recipient in need of a transplant and the potential donor. The biological sample can be any bodily fluid (e.g., blood, serum, plasma, amniotic fluid, cerebrospinal fluid, saliva, urine, or semen), tissue, or cell and the sample is tested for the presence or absence of a deletion variant either at the nucleic acid level or the antigen level using the methods described above. For organ transplants, a blood sample or a biopsy sample from the organ to be transplanted or both are preferred. For bone marrow transplants, a blood, serum, or plasma sample is preferred, although the particular of involvement of liver, intestine, and skin in typical GVHD suggests that antigens in liver, intestine, and skin are also relevant to histocompatibility.

Deletion variant, preferably common deletion variant, typing information can include a nucleic acid “type” or antigen “type” for a particular antigen identified by the methods described herein as having a common deletion variant or any combination of the antigens described in Table 1. Common deletion variant typing can also include whole genome sequences for an individual where common deletion variants can be identified and matched with potential donors based on genome sequencing and analysis as described herein. Deletion variant typing information can also include deletion variant pattern or deletion variant antigen pattern information for a subject.

An organ recipient and organ donor are said to match when the organ donor does not have any antigens that are deleted in the recipient. For histocompatibility between an organ or tissue donor and an organ or tissue recipient, one of three scenarios can occur: 1) both the recipient and the donor have a deletion variant in all copies of the gene, which prevents expression of the antigen in both the recipient and the donor; 2) both the recipient and the donor do not have a deletion variant and both express the antigen; and 3) the recipient does not have the deletion variant and expresses the antigen and the donor has a deletion variant in all copies of the gene that prevents expression of the antigen. In all of these scenarios, the immune system of the recipient would not be newly exposed to the antigen upon transplantation. For histocompatibility between a bone marrow or peripheral blood donor and a bone marrow or peripheral blood recipient, one of three scenarios can occur: 1) both the recipient and the donor have a deletion variant in all copies of the gene which prevents expression of the antigen in both the recipient and the donor; 2) both the recipient and the donor do not have a deletion variant and both express the antigen; and 3) the donor does not have the deletion variant and expresses the antigen and the recipient has the deletion variant in all copies of the gene that prevents expression of the antigen. In all of these scenarios, the immune system of the bone marrow donor would not be newly exposed to the antigen expressed by the recipient upon transplantation.

The methods described herein can be used to detect a deletion variant in a single gene or in more than one gene. For example, an individual can be typed for the presence of one, two, three, four, five, six or more common deletion variants in expressed antigens. Furthermore, an individual can be screened for deletion variants throughout her genome using whole genome sequencing techniques such as those described above (e.g., genomic hybridization to microarrays, microelectrophoresis, and single molecule sequencing). While it is preferred that two subjects are a perfect match for each and every common deletion variant tested, individuals can be ranked for immunocompatibility depending on the number of matches and the relative importance of the antigen expressed by the gene having the common deletion variant. Priority scoring systems, statistical analysis, and metrics can be used by the skilled artisan to rank the subjects for immunocompatibility. For example, an individual in need of a liver transplant would generally seek a donor having a common deletion variant type match at any, and preferably all, of the UGT2B17, UGT2B28, and GSTM1 loci, all of which are expressed in the liver, but may not be matched for common deletion variants at the OR51A2 locus, which is expressed in the olfactory epithelium. An individual in need of a kidney transplant would generally seek a donor having a common deletion variant type match at any, and preferably all, of the UGT2B28, GSTT1, and GSTM1 loci, all of which are expressed in the kidney. An individual in need of a bone marrow transplant would generally seek a donor having a common deletion variant type match at any, and preferably all, of the UGT2B17, UGT2B28, GSTM1, GSTM1, and CYP2A6 loci. Combinations of the above with any additional deletion variants either described herein or known in the art, or identified by whole genome sequencing analysis as described herein, can be used to further type the candidate transplant donor and recipients.

A transplant recipient can be screened or “typed” for deletion variants, preferably common deletion variants, in any one or more of the nucleic acids or antigens listed herein at any time after diagnosis of a disease or a propensity to develop a disease that would require an organ, tissue, blood, or bone marrow transplant. A transplant donor can be screened or “typed” for deletion variants, preferably common deletion variants, in any one or more of the antigens listed herein at any time after which the decision to donate or serve as a potential donor is made or after the donor's organ, tissue, blood or bone marrow become available. Information regarding the common deletion variant typing of the recipient and donor can be used to identify a histocompatibility match with an already identified individual (e.g., a sibling or a relative) or entered into a registry or waiting list for subjects in need of an organ or bone marrow transplant and potential donors along with additional pertinent information such as name, age, sex, race, blood type, HLA tissue type, geographic location, and urgency of the needed organ or tissue donation.

Procedures for matching transplant donors and recipients using transplant registries are known to the skilled artisan. Generally, when organs are donated, the procuring organization accesses the national transplant computer system, UNetsm, through the Internet, or contacts the UNOS Organ Center directly. In either situation, information about the donor is entered into UNetsm and a donor/recipient match is run for each donated organ. The resulting match list of potential recipients is ranked according to objective medical criteria (i.e. blood type, tissue type, common deletion variant or antigen type, size of the organ, medical urgency of the patient, as well as time already spent on the waiting list and distance between donor and recipient). Each organ has its own specific criteria.

Using the match of potential recipients, the local organ procurement coordinator or an organ placement specialist contacts the transplant center of the highest ranked patient, based on policy criteria, and offers the organ. If the organ is turned down, the next potential recipient's transplant center on the match list is contacted. Calls are made to multiple recipients' transplant centers in succession to expedite the organ placement process until the organ is placed. Once the organ is accepted for a patient, transportation arrangements are made and the transplant surgery is scheduled.

Antigen or nucleic acid typing using the deletion variants identified herein can also be used to determine the need for additional immunosuppressive medications such as purine analogs, corticosteroids, FK506, cyclosporine, rapamycin, mycophenolate mofetil, antithymocyte globulin, and anti-CD3 and anti-IL-2 receptor monoclonal antibodies during and after transplantation. For example, if the donor and recipient were not perfectly matched for antigens tested, the clinician may decide to use additional immunosuppressive medications than if donor and recipient had been a perfect match.

In addition, using the deletion variants described herein, immune rejection can also be monitored by assaying for the presence of antibodies directed against the common deletion variant antigen. Standard immunoassays using the antigen as a substrate to detect binding to antibodies present in the serum or blood sample from a subject are known in the art. Examples of kits in the art used to detect antibodies to a given antigen in serum include kits to detect Helicobacter pylori, Rubella, and cytomegalovirus.

In this example, a recipient, after transplantation, can be screened regularly for the presence of antibodies, or fragments thereof, that specifically bind any of the deletion variant antigens that are or are not matched for the donor and recipient samples. The increased presence of such antibodies as compared to a sample taken prior to transplantation is indicative of an immune response against the antigen and may suggest imminent graft rejection. In this case, the clinician can use the information to make decisions regarding the use of additional immunosuppressive medications or removal of the graft. The development of therapies for depleting such antibodies from a patient, or for masking or otherwise interfering with their ability to bind to antigen, is also contemplated in this invention.

Graft Versus Tumor Effect

An immune attack by donor-derived immune cells against cancerous host cells is frequently a desired feature of a bone marrow transplant. This “graft-versus-tumor” or “graft-versus-leukemia” effect has been an occasionally successful but highly unpredictable feature of bone marrow transplant. Bone marrow derived from individuals who are deleted for antigens that are generally expressed selectively in leukemic cells might be able to mount a graft-versus-leukemia response without causing a dangerous graft-versus-host risk to other tissues.

In this subset of bone marrow or peripheral blood transplantation, an immune response is actually desired in order to mount an attack against tumor cells present in the blood or bone marrow of the recipient. When a subject has a hematologic disorder, such as blood cell cancer, a bone marrow or peripheral blood transplant is used to introduce new marrow into the recipient's system in order to produce healthy red blood cells, white blood cells, and platelets. Bone marrow transplants are often used, for example, after high doses of chemotherapy or radiation which killing the cancer cells but also kill the patient's bone marrow.

In this example, common deletion variant typing of the nucleic acids of the invention or the antigens encoded by the invention is done to identify a bone marrow or blood donor that is not compatible with the recipient. Any one or more of the antigens or common deletion variants can be screened but it is most desirable to screen for antigens that are expressed by the cancer cells or progenitor cells. Alternatively or additionally, a whole genome sequence analysis can be performed to identify common deletion variants at a deletion mismatch loci. A donor is identified as incompatible with the recipient if the donor has a deletion variant in all copies of the gene that prevents expression of the antigen and the recipient does not have the deletion variant and expresses the antigen. Once a histoincompatible donor is identified for the recipient, the transplant is performed and desirably, results in an immune attack mounted by the donor's transplanted immune cells against the remaining cancer or disease cells in the host recipient. This desired outcome of transplantation is termed graft versus tumor and not only provides healthy blood cells to the patient but also aids in the treatment of the cancer by killing the remaining cancer cells.

Maternal/Fetal Immunocompatibility

The methods of the present invention are also useful for screening individuals for immunocompatibility to diagnose and understand maternal/fetal incompatibility issues that may contribute to spontaneous abortion or miscarriage. In some cases, fertility issues arise not because of fertility problems but because of immunocompatibility issues between the mother and the prospective father or sperm donor. One common example of such a case occurs when a mother is Rh negative and her partner is Rh positive. Rh factor is a protein present in the red blood cells of most people, capable of inducing intense antigenic reactions. If the mother has an Rh antibody titer after sensitization during a previous pregnancy or due to a previous incompatible transfusion, and the fetus is Rh positive, then the mother's immune system can mount an attack against the fetal cells expressing the Rh factor. Such an attack can result in spontaneous abortion or many lifelong complications for the baby before and after birth. Pregnant women or women interested in conceiving are often tested for the presence of antibodies for Rh as are fetuses in women who are Rh negative.

Despite this understanding of Rh compatibility issues, many spontaneous abortions and fertility problems still occur as a result of incompatibility of antigens that have not yet been identified. Using the methods of the present invention, a woman and a prospective man wanting to conceive can be tested for any one or more of the common deletion variants of the invention. Such typing can occur at the DNA level (either whole genome sequencing or to identify the presence or absence of known deletion variants) or using antigen typing for common deletion variant antigens other than MHC, Rh factor, or blood type. Antigen typing can occur as a preliminary screen or after fertility problems or one or more spontaneous abortions have occurred. Similarly, a woman intending to use a sperm donor can be screened and the sperm can be screened for deletion variants or expression of the deletion variant antigens encoded by the polymorphic genes. In the case of known incompatibility, the fetus can also be tested. Similarly, a woman undergoing in vitro fertilization could have several embryos tested for histocompatibility with her, to ensure that a histocompatible embryo is implanted and thereby maximize the probability of a successful pregnancy. Information gained from antigen or common deletion variant typing can be used to understand fertility issues, to identify problems with potential partners, or to monitor an at risk fetus when incompatibility is known.

For histocompatibility between a woman and a prospective father or sperm donor or an embryo or fetus, one of three scenarios can occur: 1) both the mother and the father, sperm, embryo or fetus have a deletion variant, preferably a have a deletion variant in all copies of the gene, which prevents expression of the antigen in both the recipient and the donor; 2) both the mother and the father, sperm, embryo or fetus do not have a deletion variant and both express the antigen; and 3) the mother does not have the deletion variant and expresses the antigen and the father, sperm, embryo or fetus has a deletion variant in all copies of the gene that prevents expression of the antigen. In all of these scenarios, the immune system of the mother would not be newly exposed to the antigen upon transplantation.

In one example, a pregnant woman presents at her OB/GYN office for a prenatal visit. Routine blood work determines that she has one or more common deletion variants resulting in non-expression of the encoded antigen. Examples of particular deletion variants that are useful in this method include UGT2B28, UGT2B17, and LCE3C, all of which are expressed in the placenta. Her partner does not have the common deletion variant and expresses the antigen. The pregnant woman is then further tested to determine if she has a serum antibody titer to the antigen. Fetal DNA or antigen typing using amniotic fluid can also be performed. If the fetus is determined to lack the common deletion variant or express the antigen, further monitoring of the fetus by the clinician or by sonography or amniocentesis can be performed. However, if the fetus is determined to have the common deletion variant, the fetus is judged to be at low risk for immune attacks by the maternal immune system and can be followed by non-invasive procedures such as sonography.

Combination Screening Methods

Although the methods described herein are effective for determining immunocompatibility between individuals, they can also be combined with additional known screens and tissue typing methods for the identification of compatible or incompatible individuals. Such methods are known in the art and include blood type matching, Rh factor typing, and HLA typing, both of which are known in the art. When immunocompatibility is desired, individuals matched for antigens can also be screened (either prior to or after antigen screening) for matching blood types and matching HLA types. When immunoincompatibility is desired, individuals that are identified as having different antigens types can also be screened (either prior to or after antigen screening) for the presence or absence of distinct blood types and HLA types.

For blood typing, an individual with Type A blood is compatible with an individual with Types A or O. An individual with Type B blood is compatible with an individual with Types B or O. An individual with Type O blood is only compatible with an individual with Type O. An individual with Type AB blood is compatible with an individual having any blood type. Blood types can also be measured for compatibility of Rh factor.

For HLA typing, the screen can include any number of the proteins encoded by the HLA region and generally includes from one to six of the proteins. The polymorphic proteins encoded by the HLA region have been designated HLA-A, -B, -C,-DR,-DQ, and -DP. HLA-A, -B, and -C consist of a single polymorphic chain. HLA-DR, -DQ, and -DP proteins contain two polymorphic chains, designated alpha and beta. These D-region proteins are encoded by loci designated DRA, DRB1, DRB3, DRB4, DQA1, DQB1, DPA1, and DPB1. (See Schwartz, Ann. Rev. Immunol. 3:27-261, 1985.) The products encoded by the polymorphic HLA loci are most commonly typed by serological or nucleic acid based typing methods. See for example, U.S. Pat. No. 6,194,147 for a description of methods for HLA typing.

Of the many HLA antigens, the National Marrow Donor Program (NMDP) sets minimum matching levels that must be met before a donor or cord blood unit from the NMDP Registry can be used for a transplant. These minimum requirements are based on research studies of transplant outcomes. The HLA antigens that are looked at for these minimum requirements are called HLA-A, -B and -DRB1. One set of these three antigens is inherited from the mother and another set is inherited from the father. This makes a total of six antigens to match. For cord blood units, the NMDP requires a match of at least four of these six HLA antigens. For adult marrow or peripheral (circulating) blood cell donors, the NMDP requires a match of at least five of these six HLA antigens.

Potential donors and recipients can also be tested for crossmatching in which the recipient's blood and the potential donor's blood are place together in a test tube and examined to see if there is cell death. If all the cells survive without death of the donor's cells, there is a negative crossmatch, which is indicative of immunocompatibility of the individuals. If the cells of the donor begin to die, a positive crossmatch results, which is indicative of immunoincompatibility.

Tolerization

For any of the immunocompatibility testing methods where compatibility is desired, if two subjects are found to be immunoincompatible due to the presence in one subject of a deletion variant in all copies of the gene that is not present in another subject (e.g., an organ donor and a recipient or a potential mother and father trying to conceive), but a transplant or fertilization must still take place between the two subjects, methods for tolerizing the subject having the common deletion variant and therefore lacking the expressed antigen can be used to reduce the risk of organ rejection or spontaneous abortion.

Tolerization regimens are intended to prepare the immune system of an individual (e.g., organ recipient or prospective mother) to accept an antigen that is not expressed in that individual due to a polymorphic deletion. For example, an individual awaiting an organ transplant could be treated to facilitate acceptance of antigens that are not expressed in that individual. In another example, if prospective parents are not compatible because the prospective mother does not express one or more of the antigens encoded by the common deletion variants and the prospective father does, the prospective mother can be treated to tolerize her to the presence of the antigen that may be expressed on the fetus.

Tolerization can be achieved through any gene therapy or protein therapy regimens known in the art for delivery of an antigen or a nucleic acid encoding an antigen to the individual in need of tolerization. The purified protein or nucleic acid encoding the antigen can be delivered directly to a target organ or systemically.

For protein therapy, purified forms of the antigen used for tolerization can be purchased from a commercial source or can be produced by recombinant methods known in the art (see, for example, Sambrook et al., Molecular Cloning: A Laboratory Manual, Vols. 1-3, Cold Spring Harbor Laboratory Press, 3 ed., 2001, or F. Ausubel et al., Current Protocols in Molecular Biology (Green Publishing and Wiley-Interscience: New York, 1987) and periodic updates.

The desired antigen can also be delivered via a nucleic acid encoding the antigen. The nucleic acid can be any nucleic acid (DNA or RNA) including genomic DNA, cDNA, and mRNA encoding the antigen. Methods for nucleic acid therapy are known in the art and can be found, for example, in Sambrook et al., supra, Ausubel et al., supra, and Watson et al., Recombinant DNA, Chapter 12, 2d edition, Scientific American Books, 1992).

In gene therapy applications, genes are introduced into cells in order to achieve in vivo synthesis of a therapeutically effective genetic product. “Gene therapy” includes both conventional gene therapy where a lasting effect is achieved by a single treatment, and the administration of gene therapeutic agents, which involves the one time or repeated administration of a therapeutically effective DNA or mRNA. Standard gene therapy methods typically allow for transient protein expression at the target site ranging from several hours to several weeks. Re-application of the nucleic acid can be utilized as needed to provide additional periods of tolerization.

An additional method for tolerizing immune cells from one individual to a known antigen is to “immunodeplete” those cells which bind to a particular antigen, or which bind to peptide fragments presented on cell surfaces by the MHC. Methods for immunodepletion are known in the art and are reviewed, for example, in Blazar and Murphy, Philos Trans R Soc Lond B Biol Sci. 360:1747-67 (2005).

### EXAMPLES

### Example 1

### Identification of Aberrant Genotype Patterns Across the Genome

The locations of common deletions in the human genome are largely unknown, as is the best way to determine the association of such variants with disease. To address these questions, we developed an approach for using the HapMap to discover, localize, and analyze common deletion variants. We found hundreds of deletion variants, 1 kb-745 kb in size, including more than 100 common deletions that were observed as homozygous deletions. Ten of these common deletion variants remove the coding regions of expressed genes thought to contribute to drug response, olfaction, and sex steroid hormone metabolism; the gene deletion variants also explained variation in gene expression at these loci. Most common deletions appear to result from ancestral mutations that have been inherited by descent; they are in linkage disequilibrium with nearby single-nucleotide polymorphisms (SNPs), such that their association to disease could be discovered in whole-genome association studies.

SNPs have long been appreciated as common, potentially phenotype-causing genetic variants and as markers for other, undiscovered variants via linkage disequilibrium. Genome-wide SNP discovery efforts, and the construction of a map of human SNP variation (HapMap consortium), allow for the use of whole-genome SNP genotyping to discover common ancestral mutations that affect disease risk.

Recently, it has been recognized that structural variation—including duplications, deletions, and inversions—is common and extensive. (See, for example, Sebat et al., Science 305:525-8 (2004); Iafrate et al., Nat. Genet. 36:949-51 (2004); Tuzun, et al., Nat. Genet. 37:727-32 (2005); and Sharp et al., Am. J. Hum. Genet. 77:78-88 (2005)). Of all forms of structural rearrangement of a locus, the form with the most obvious potential functional relevance is that which removes the DNA sequence altogether. However, little is known about the location of common deletion polymorphisms on the scale of specific exons and regulatory elements; even less is known about which deletion variants may be sufficiently common to appear as homozygous deletions in many individuals.

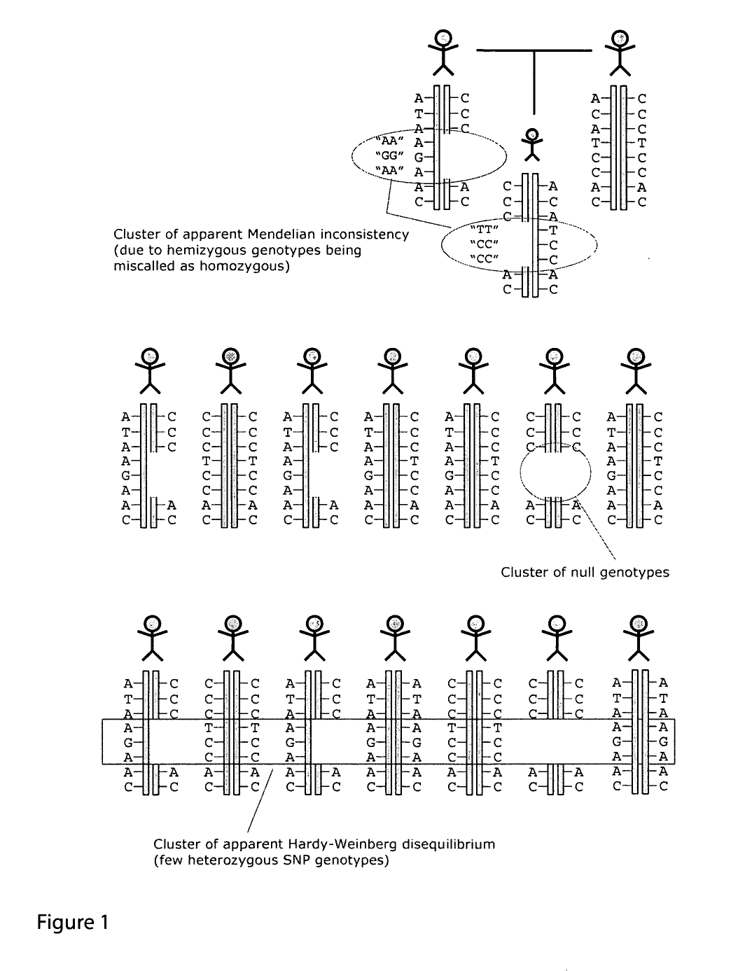

To identify, catalog, and enable study of deletion variants across the human genome, we set out to develop and validate a method for discovering deletions from SNP genotypes. We hypothesized that a segregating deletion would leave “footprints” in SNP genotypes, including null genotypes, apparent deviations from Mendelian inheritance, and apparent deviations from Hardy-Weinberg equilibrium (FIG. 1). This is complicated by the fact that technical artifacts and genotyping errors also give rise to these three “failure modes” at individual markers. In fact, because of the likelihood that such deviations are errors, genotype assays have long been discarded from medical genetic studies when such failures are observed.

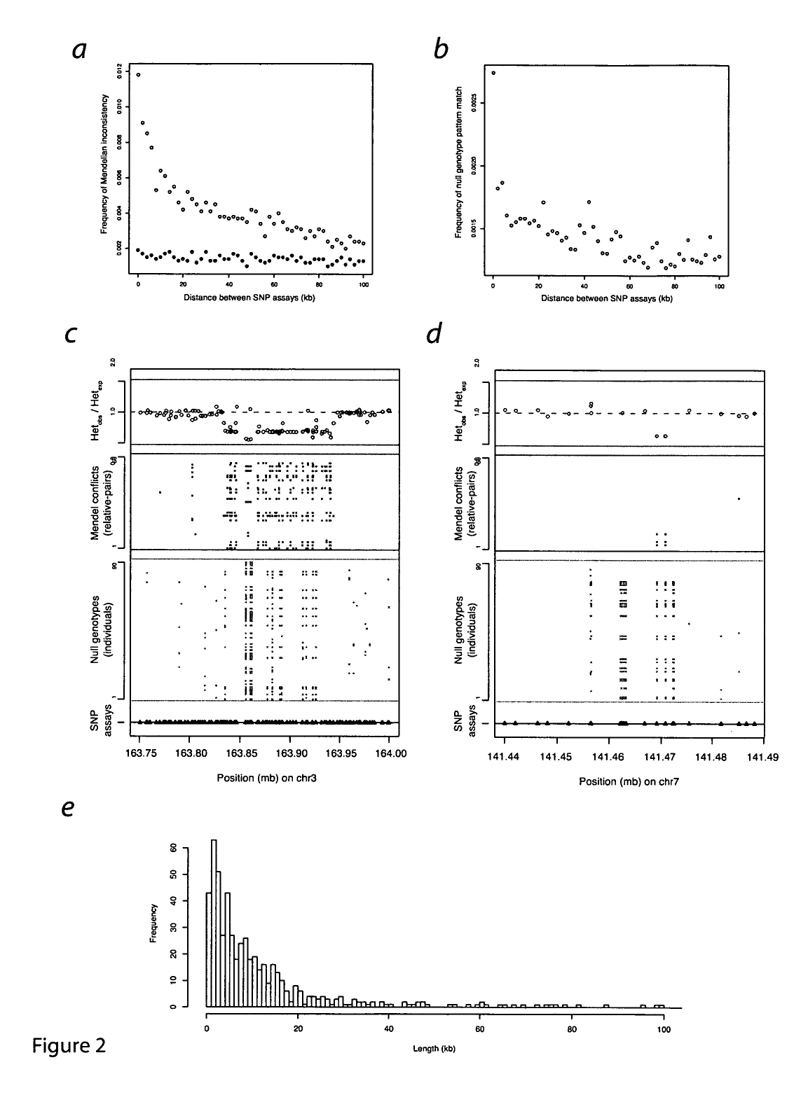

To determine whether a subset of “failed” SNP genotyping assays in the HapMap data might reflect structural variation, we asked whether such failures are physically clustered in a manner that is specific to individuals. Consistent with this hypothesis, the rate of Mendelian-inconsistent genotypes was elevated near other Mendelian-inconsistent genotypes in the same individual (regardless of whether the same genotyping platform was used for both assays), but was unrelated to Mendelian inconsistencies in other individuals (FIG. 2A). A similar relationship was observed for null genotypes (FIG. 2B). Thus, such clustering is a property of individual variation in local sequence, rather than the local sequence per se.

We used data from the International HapMap Project to identify clusters of aberrant genotype patterns across the genome. We used the unfiltered genotypes from release 16 of the HapMap, which we downloaded from http://hapmap.org. These consisted of separate genotype files for four population samples: 90 CEPH individuals (30 trios) of European ancestry; 90 individuals (30 trios) of Yoruban ancestry sampled in Ibadan, Nigeria; 45 unrelated individuals of Han Chinese ancestry sampled in Beijing; and 45 unrelated individuals of Japanese ancestry sampled in Tokyo. The population samples are described in detail in Altshuler et al. Nature 437:1299-1320 (2005)). We combined the data from the Chinese and Japanese population samples and thereafter treated the data set as three population samples of 90 individuals each.

A complication is that this data had been generated at ten different genotyping centers, using seven different genotyping technologies, each of which showed distinct rates of each type of “failed assay.” We noted that the background rates of Mendel failure, null genotypes, and Hardy-Weinberg disequilibrium differed greatly from technology to technology, and even for the same technology when used by different centers. Furthermore, there were many sample-by-batch interactions, in which particular samples were associated with elevated rates of null genotypes or Mendel failures in particular experimental batches. To distinguish physically clustered patterns of aberrant genotypes from sporadically appearing patterns, we developed a set of statistical thresholds, tailored to each genotype pattern, genotyping center, and genotyping technology, for identifying significantly clustered patterns.

Because we sought to identify multi-assay patterns in the data from independent genotype assays, we did not combine data from multiple assays that potentially used the same sequence features for amplification, labeling, or restriction digest. Thus, we excluded all the Perlegen assays, because the use of 10-kb amplicons on that platform potentially caused long-range patterns of aberrant genotypes wherever an undiscovered SNP altered either primer-binding site. We also excluded data from any experiments whose batch structure corresponded to physical regions of the genome, because this design potentially allowed batch-specific experimental artifacts to appear as regional patterns in the data.

We looked for clustering of aberrant genotype patterns in each of the populations separately as described below.

Null Genotypes

For each genotype assay and population sample, we defined the “null genotype pattern” of that assay as the binary vector (length 90) of null genotype calls across the 90 individuals in that population sample. For each such pattern that was observed on any genotyping platform, we considered each pattern together with its close neighbors (R²>0.8) in pattern space. (This fuzzy clustering was necessary because genotype assays do not consistently obtain 100% complete calls, even in euploid samples.) We determined the background frequency of that set of patterns on the combination of genotyping technology, genotyping center, and (wherever possible) on the specific experimental batch in question. Using that background frequency, we defined a statistical threshold for clustering by finding numbers x and y such that the binomial probability of observing 2 occurrences in x physically consecutive assays, or 3 occurrences in y physically consecutive assays, was sufficiently small that, after testing (num_patterns×num_assays) hypotheses, we would expect fewer than two chance discoveries per platform. We identified all genomic segments (runs of two or three examples of the pattern) where the clustering of this pattern exceeded the statistical threshold, and clustered any segments that overlapped.

Mendel Failures

For each genotype assay and population sample (CEPH and Yoruba samples only), we defined the “Mendel failure pattern” of that assay as the binary vector (length 60) of null genotype calls across the 60 parent-offspring pairs in that population sample. For each such pattern that was observed, we considered each pattern together with its close neighbors (R²>0.8) in pattern space. This fuzzy clustering was desirable because the same deletion segregating in a population can give rise to non-identical patterns of Mendel failure at different SNPs, due to the fact that the non-deletion SNP haplotypes that are segregating in a trio (whose conflicts result in the Mendel conflicts) may not disagree at all SNPs.

Assessment of Clustering of “Failure Profiles”

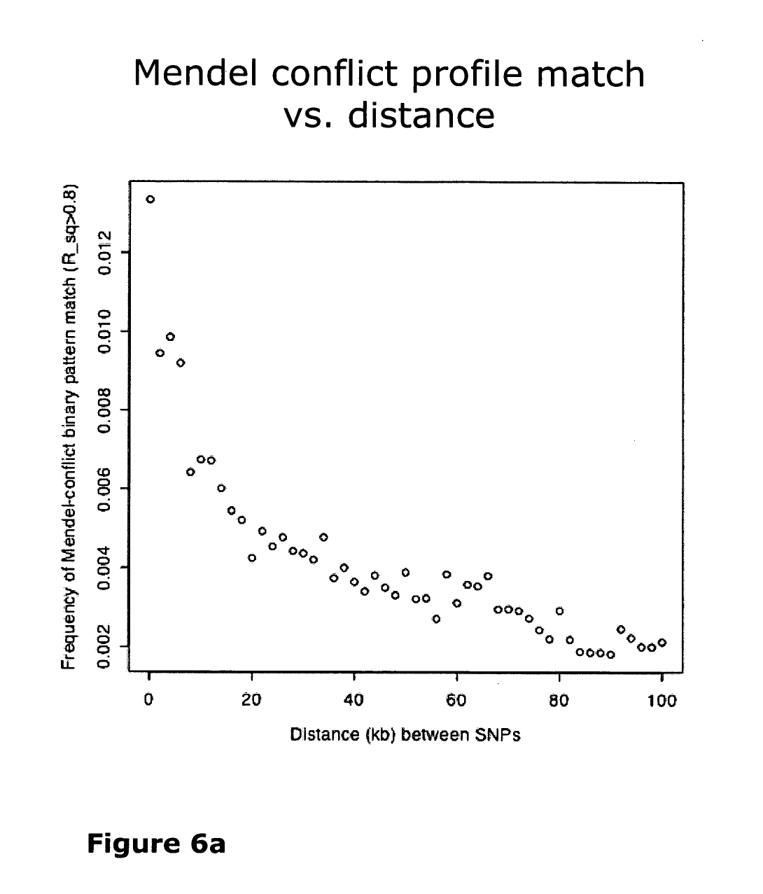

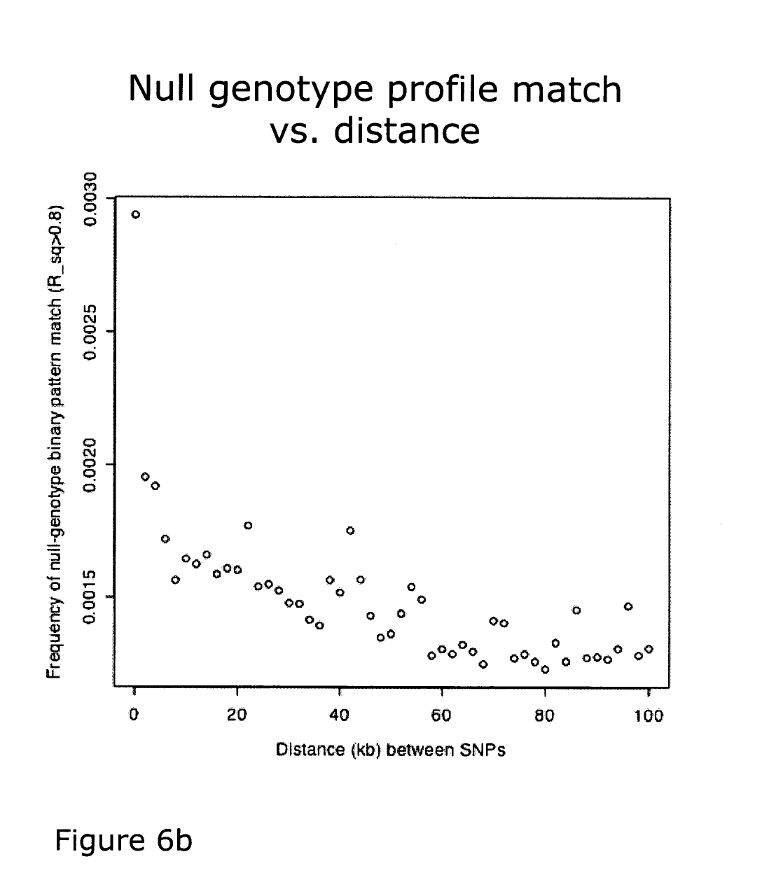

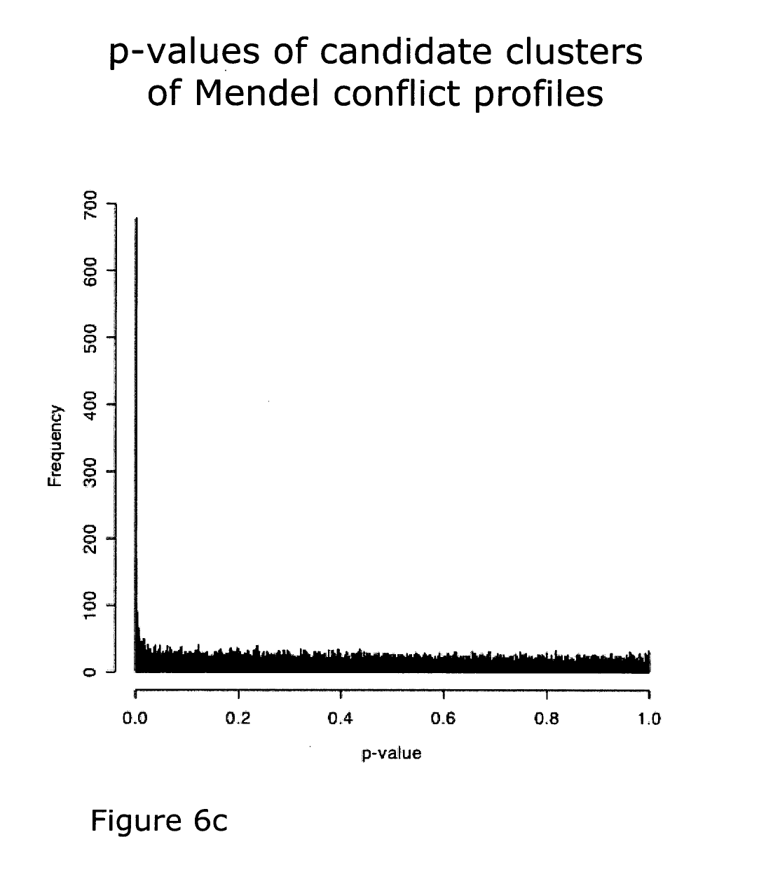

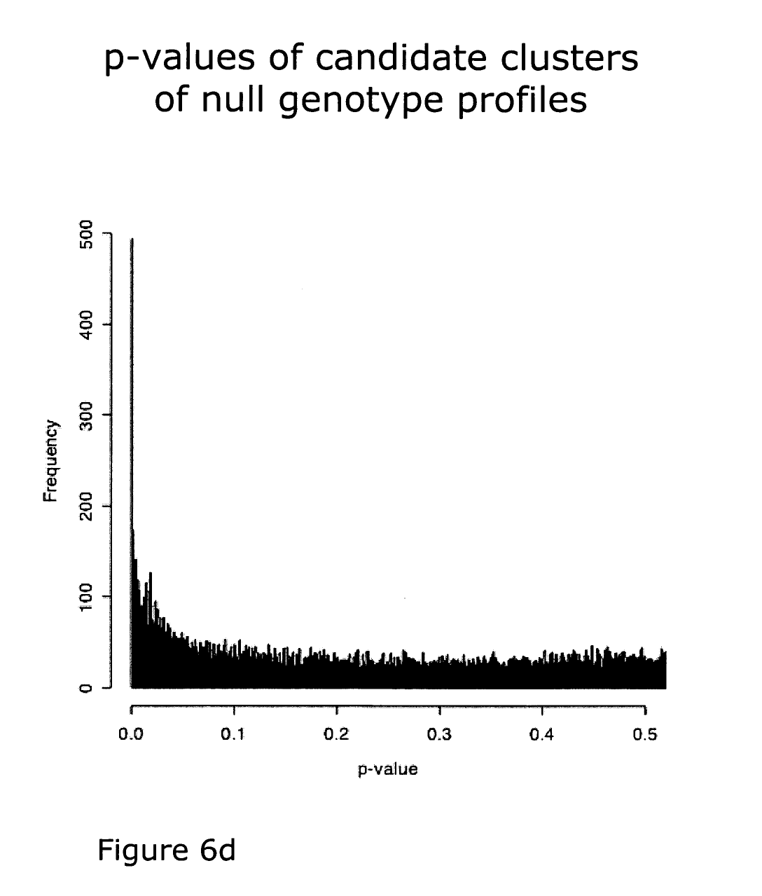

For both Mendel failure profiles and null genotype profiles, we observed that highly similar (R²>0.8) profiles tended to be physically clustered in the genome. More specifically, we observed that the probability of observing a “match” to any particular profile was a decreasing function of physical distance from that profile, even when we considered only pairs of SNP assays that were typed using different technology platforms (FIGS. 6A and 6B).

The Phase I HapMap data was produced by ten different genotyping centers, with each chromosome arm primarily genotyped by one particular center (HapMap Consortium, Altshuler et al. Nature 437:1299-1320 (2005)). Approximately 120 thousand SNP assays were performed by centers outside of their primary regions, or on genome-wide platforms such as Affymetrix 100K SNP arrays, allowing cross-platform analyses like those in FIG. 2A and FIGS. 6A and 6B. However, because the overwhelming majority of assays in any particular region were performed at a single genotyping center, any effort to identify local multi-marker features in the HapMap data must of necessity compare many SNP assays that were produced by the same center and genotyping technology. It was therefore critical to control for center- and platform-specific patterns in the data. An initial survey of the data suggested that such patterns were potentially abundant; for example, the background rates of Mendel failures varied from center to center (with additional examples of center-by-sample interaction), and particular DNA samples tended to have low genotype conversion rates (high null genotype rates) at particular centers. Thus, it was important that the clustering of each pattern be assessed in a format that controlled for such center- and platform-specific patterns in the data.

We therefore analyzed the data from each genotyping center separately. For each genotyping center, we first ordered all of the SNP assays from that center by genomic position. For each pattern (clustered set of highly similar profiles) that was observed multiple times, we determined that pattern's background frequency at that center, and wherever possible on the specific experimental batch in question. (Batch information was obtained from the International HapMap Consortium.) We then analyzed the physical distribution of all observations of that pattern relative to all of the SNP assays from that center (ordered by genomic position). A list of “candidate clusters” was determined by considering every consecutive pair and consecutive trio of observations of that pattern, together with any other, intervening SNP assays from that center. To assess the tightness of each such candidate cluster, a “clustering p-value” was calculated to assess the probability of observing a cluster at least as tight (in consecutive-assay space) as that cluster, given (i) the background frequency of the pattern, (ii) the number of SNP assays spanned by the cluster, and (iii) the total number of SNP assays performed by that center. The distribution of these p-values is shown in FIGS. 6C and 6D. These figures show a generally uniform distribution of p-values from zero to one, but with an excess of very low p-values. The region of excess low p-values can be thought to identify a set of candidate clusters in which the alternate hypothesis (non-random degree of clustering) is likely to be true; this region is separated by a “knee” from the rest of the distribution, which is organized as a generally uniform distribution (FIGS. 6C and 6D). We chose a significance threshold for promoting potential clusters, based on the goal of capturing as many true discoveries as possible, while maintaining a false discovery rate of no greater than 10% of all discoveries. This required selecting a significance threshold somewhat to the left of the “knee” in the distribution, where the height of the distribution was at least ten times greater than the average height of the distribution to the right of the knee. In FIGS. 6C and 6D, this corresponds to the leftmost bars of the histogram (a p-value of 1.8×10⁻³ for Mendel patterns, and 9×10⁻⁴ for null-genotype patterns). The large additional region of excess low p-values, not captured by this threshold, suggests a significant type II error rate (because no gold-standard data set exists, the true type II error rate is not known). As the density of markers in the HapMap increases during subsequent phases of the project, many of the clusters in this region may be either confirmed by additional assays (increasing the clustering and promoting the cluster beyond the threshold) or not confirmed (reducing the level of clustering and increasing the p-value).

We clustered all overlapping genomic segments that were identified by this analysis, into 702 genomic loci.

We were concerned that multiplexed batches of SNP assays that were performed together could also give rise to potential patterns in the data, which (if distributed non-randomly in genomic space with respect to that center's other SNP assays) could give rise to potential batch artifacts. We therefore excluded those clusters that consisted entirely of SNP assays from the same experimental batch. This resulted in a set of 541 predictions.

Hardy-Weinberg Disequilibrium

We observed that a deletion tended to reduce the ratio of observed heterozygosity to expected heterozygosity (het_{obs}/hetₑₓₚ) by a uniform amount (FIG. 2C), this amount being determined by the population frequency of the deletion haplotype. We thus looked for genomic regions in which (het_{obs}/hetₑₓₚ) consistently fell underneath some cutoff (we used cutoffs of 0.7 and 0.4). We included only those assays with a minor allele frequency greater than 10 percent. For each genotyping platform, we determined the background frequency of assays for which (het_{obs}/hetₑₓₚ) was less than the cutoff, and used this frequency to determine statistical thresholds for clustering as described above.

Wherever the resulting genomic segments overlapped with clusters of Mendel failure or null genotypes as discovered above, we clustered those segments together. (Because heterozygosity can show regional correlations due to haplotype structure, selection, and potentially duplicated sequence, we did not promote loci based on (het_{obs}/hetₑₓₚ) alone unless confirmed by one of the other lines of evidence; however, the (het_{obs}/hetₑₓₚ) deviations were useful for extending clusters discovered by Mendel failures, because the Mendel failures themselves may not be observed at every marker in the deleted region (FIG. 2C).)

More specifically, we defined as the “failure profile” of an assay its pattern of Mendel failure across the 60 pairs of relatives in a population, its pattern of null genotypes across the 90 individuals in a population, and its deviation from the expected level of heterozygosity in that population. We looked for regions of the genome in which highly similar “failure profiles” appeared at nearby markers (FIGS. 2C and 2D), to an extent not explained by center-, platform-, or batch-specific patterns in the data. To assess the statistical significance of each candidate cluster, we calculated the binomial probability of observing each pattern n times in m markers (based on the empirically observed rate for each platform). We identified a candidate deletion when that probability was smaller than a threshold expected to result in fewer than 20 false discoveries across the genome.

Using these methods we identified 541 candidate polymorphic deletions 1-200 kB in size (as shown in Appendix A). 120 of these loci generated null genotypes in multiple individuals, suggesting the existence of common, homozygous deletions. More than 90% of the discovered deletion variants were novel. Half of these loci were 1-7 kb in size and were therefore not detectable by earlier approaches; 98% were 1-30 kb in size and would have had little chance of detection by commonly used hybridization-based approaches.

It was critical to validate the presence of segregating deletions at the predicted sites, given their origin in data that fails typical quality control standards and the statistical nature of the inference. We used four methods: fluorescent in situ hybridization (FISH), two-color fluorescence allele-intensity measurements, PCR amplification, and comparison to previous work. These methods are described below in the Materials and Methods section.

First, we performed fluorescent in situ hybridization (FISH) on four candidate deletions that completely contained available FISH probes. The FISH assays confirmed the existence of segregating deletions at each site, and confirmed their Mendelian inheritance wherever suitable cell lines were available (FIG. 3A).

Second, we examined two-color fluorescence data from the assays that had been used to genotype SNPs on chromosomes 4q, 7q, and 18p at the Broad Institute. Specifically, this method associates a quantitative fluorescence signal with each allele at each typed SNP in each individual. At most SNPs, individuals' fluorescence-intensity measurements cluster into two or three discrete groups corresponding to homozygous and heterozygous genotypes. At SNPs under 15 candidate deletion loci, fluorescence intensity data instead clustered into as many as six groups (FIG. 3B). When we compared these measurements to imputed genotypes for individuals with hemizygous and homozygous deletions, these segregated into the observed clusters (FIG. 3B).

Third, we selected 60 loci for which the pattern of genotypes suggested the existence of multiple individuals with homozygous deletions, and confirmed the existence of homozygous deletions at 51 of these loci by PCR assays that failed in the suspected homozygous-null individuals but succeeded in all other individuals tested (FIG. 3C and Tables 2a and 2b).

Fourth, quantitative PCR was performed in all 269 HapMap DNA samples for 11 candidate deletions that overlapped the coding exons of genes (described below) and were discovered in many individuals: at 10/11 loci, three discrete clusters were observed, identifying individuals with 0, 1, and 2 gene copies (FIG. 3D).

TABLE 2a
Validation of candidate deletion variants.
Experimental validation

| | | | Variants | Variants |
|---|---|---|---|---|
| Technique | Candidates screened | Criterion applied | screened | confirmed |
| FISH | 5 large candidate variants that | Absence of FISH signal in the specific individuals predicted to | 5 | 5 |
| | cover available FISH probes | harbor the deletion variant (but not in control individuals) | | |
| Two-color, | All 17 candidate common | Observation of extra genotype classes, well-separated from | 17 | 15 |
| allele-specific | variants on 4q, 7q, 17p | others clusters at at least one SNP, that contain all individuals | | |
| fluorescence | that covered at least 3 | predicted to be aneuploid at locus. | | |
| | HapMap SNPs typed at | | | |
| | the Broad Institute | | | |
| PCR | 60 candidate variants | Confirmation of predicted pattern of successful and | 60 | 51 |
| | predicted to be homozygous | unsuccessful amplification across 12-24 individuals including at | | |
| | null in at least two individuals | least 2 with each predicted result | | |
| Quantitative PCR | 11 candidate common | Clustering of measurements of DNA copy-number into three | 11 | 10 |
| | variants that affected the | discrete groups | | |
| | coding exons of genes | | | |

TABLE 2b
Comparison to earlier work.

| Earlier approach | Ref. | Variants considered | Criterion applied | Confirmations |
|---|---|---|---|---|
| ROMA | 1 | 55 “copy number polymorhpisms” (potentially | Aberrant SNPs spanned at least 20% of the | 4 |
| (Sebat et al., 2004) | | deletions or duplications) | region identified earlier | |
| BAC array CGH | 2 | 255 “large copy number variants” (potentially | Aberrant SNPs spanned at least | 3 |
| (Iafrate et al., 2004) | | deletions or duplications) | 20% of the BAC probe identified earlier | |
| BAC array CGH | 3 | 119 “copy number polymorhpisms” (potentially | Aberrant SNPs spanned at least 20% | 6 |
| (Sharp et al., 2005) | | deletions or duplications) | of the BAC probe identified earlier | |
| Fosmid end pair | 4 | 102 deletion variants | Aberrant SNPs fell completely inside the | 28 |
| sequencing | | | fosmid(s) identified earlier | |
| (Tuzun et al., 2005) | | | | |

We also tested an additional 56 loci that were not among our core predictions, but met a more-relaxed set of statistical thresholds; the confirmation rate among these other candidate variants was considerably lower, suggesting that relaxation of the statistical thresholds would be unwarranted.

Finally, we compared the locations of the candidate deletions to results from an earlier study, in which the approximate genomic locations of 102 candidate deletions in a single individual were discovered by the existence of fosmid end pair sequence reads from that individual that map more than 48 kb apart on the reference human genome sequence. (Tuzun et al., supra). Twenty-eight of our candidate deletions resided within these fosmids; in each case, the location of the aberrant genotypes further refined the localization of the deletion variant.

In sum, 90 predicted deletion variants (including 68 of 120 predicted common homozygous deletions) were validated by one or more of these approaches. Based on the experimental results, we estimate that 15% of the still-untested candidate deletion loci may be false positives.

We found thirteen genes for which exons were deleted at an appreciable frequency (Table 1). Of these genes, eight were observed as homozygous deletions. These common gene deletion polymorphisms included two genes involved in the metabolism of sex steroid hormones (UGT2B28 and UGT2B17). Common deletions also removed two genes encoding olfactory receptors (OR51A2 and OR4F5) and three genes (CYP2A6, GSTT1, and GSTM1) with roles in detoxification and drug metabolism. (For information on previously identified deletions in some of these genes see Seidgard et al., Proc. Natl. Acad. Sci. 85:7293-7297 (1988), Nunoya et al., Pharmacogenetics 8:239-249 (1998); and Pemble et al. Biochem. J. 300 Pt1:271-276 (1994).)

To assess the frequencies and inheritance of these gene deletions in different populations, we developed quantitative PCR assays for accurately genotyping individuals as carrying 0, 1, or 2 gene copies, and used these assays to successfully genotype eight of the ten gene deletion variants in all the HapMap individuals (Table 1). The resulting genotypes showed Mendelian inheritance, Hardy-Weinberg equilibrium, and expected transmission rates, suggesting that each behaves as a stable, heritable genetic variant. The gene deletion variants were observed in individuals of European, Yoruba, and Chinese and Japanese ancestry, though the frequency of each deletion varied from population to population (Table 1).

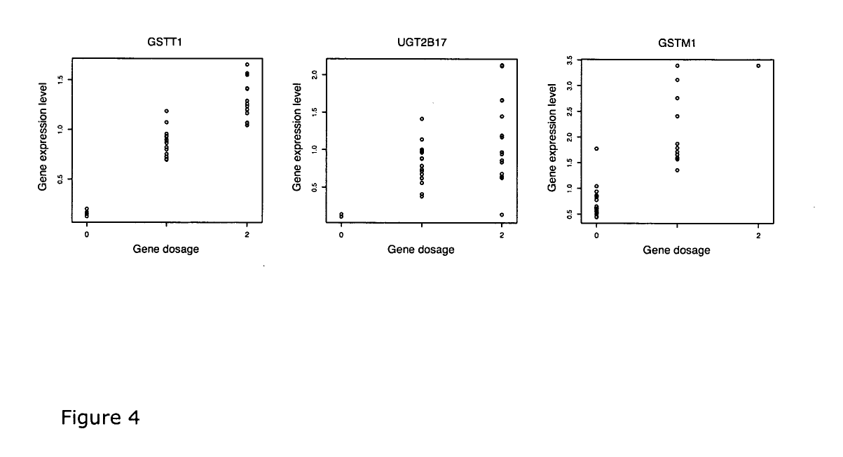

Assessing functional relevance requires testing for association to phenotype. A simple phenotype is the level of expression for each transcript. Based on global profiles of gene expression in a subset of the samples, we found that three commonly deleted genes (Table 1) are expressed at appreciable levels in the lymphoblastoid cell lines used to measure individual variation in gene expression. (Monks et al., supra and Morley et al., Nature 430:743-747 (2004)). We compared published expression measurements from these cell lines to deletion genotypes that we obtained experimentally. Variation in gene dosage explained respectively 88%, 26%, and 75% of the observed variation in expression of the three genes (FIG. 4); individuals with one copy showed 30%, 35%, and 38% less expression respectively than individuals with two gene copies. Individuals homozygous for deletion variants showed little or no expression. Individuals with one gene copy showed less expression than individuals with two gene copies, suggesting that feedback regulation had not normalized transcript level.

For medical genetics, a key question is whether one must discover each deletion variant in every patient, using dedicated technology, or can rely on linkage disequilibrium by using nearby SNPs as proxies for common deletions. The answer to this question depends on the linkage disequilibrium properties of common deletion variants: if common deletion of a locus is due to recurrent mutation there, then deletions must be discovered independently in every patient; if common deletion of a locus results from an ancestral mutation that has been inherited by descent, then it will often segregate on an ancestral haplotype and be in linkage disequilibrium with nearby SNPs.

In addition, to the extent that deletions result from unique ancestral mutational events, they will often be in linkage disequilibrium with nearby SNPs, and ancestral SNP haplotypes can serve as proxy in disease studies as well as immunocompatibility assays.

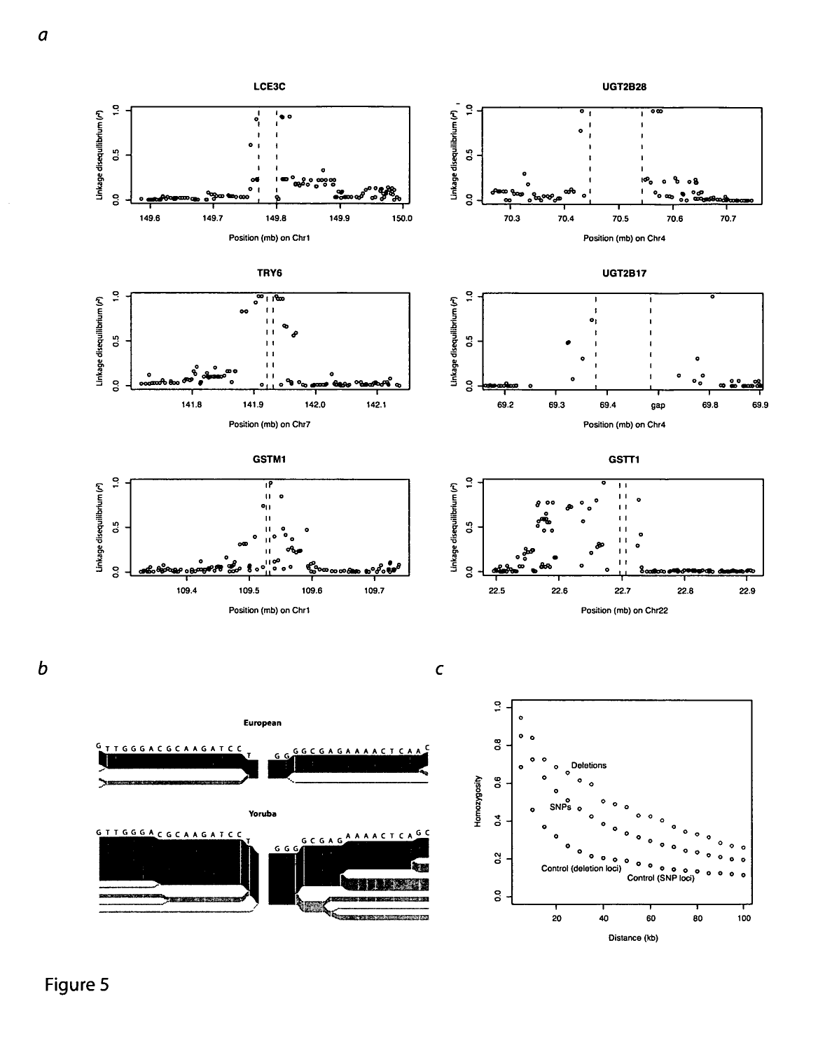

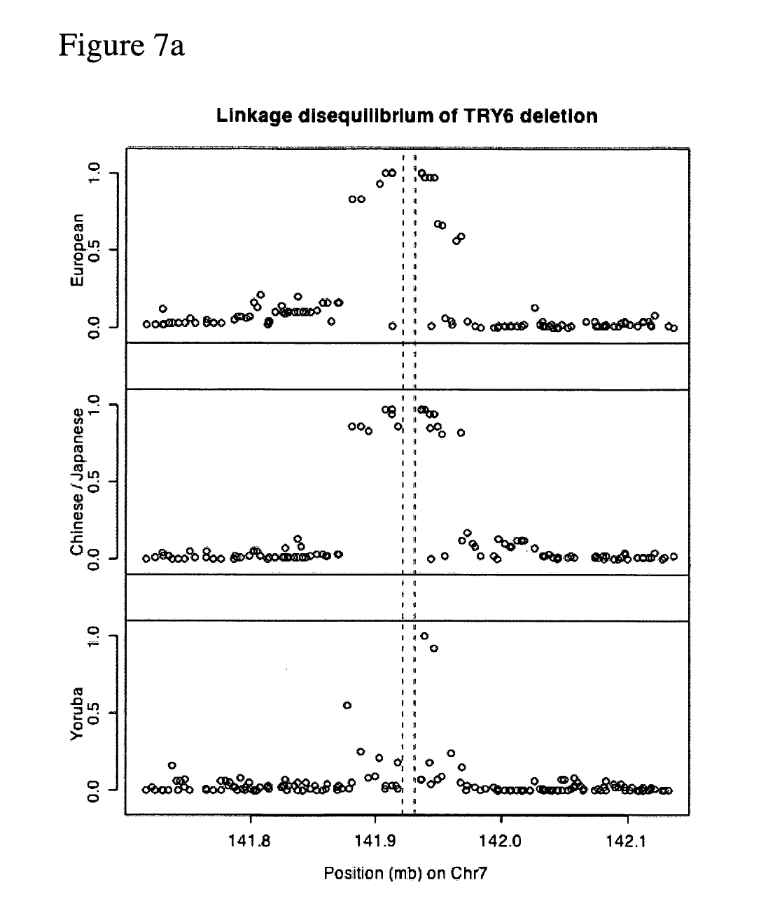

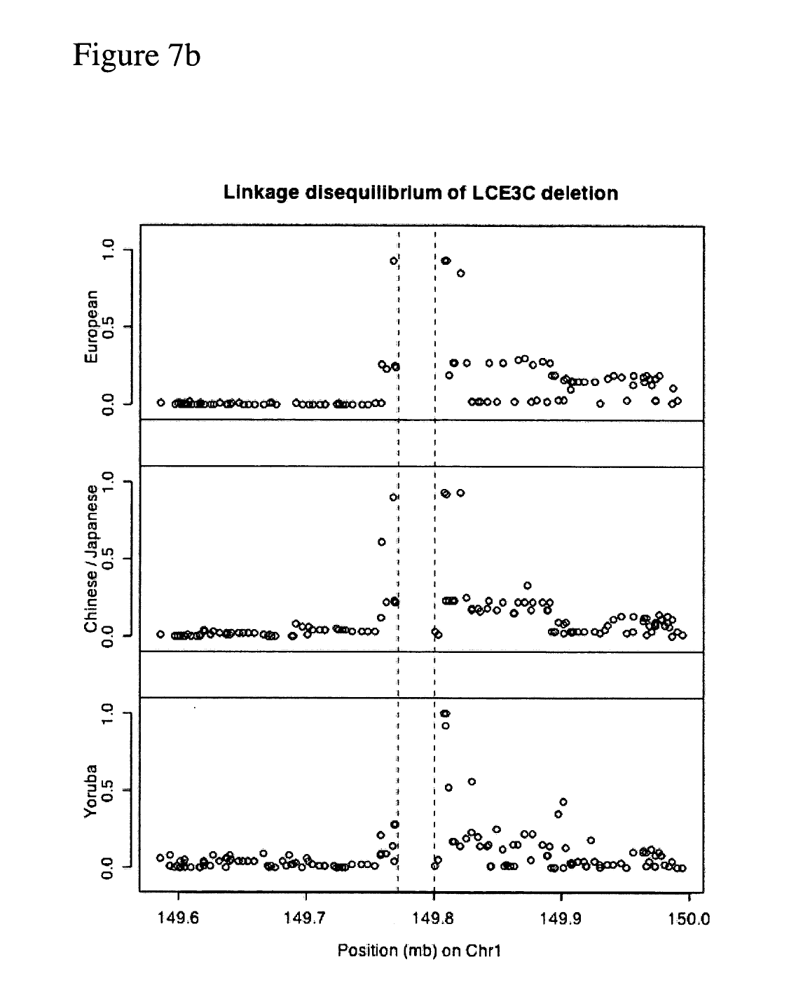

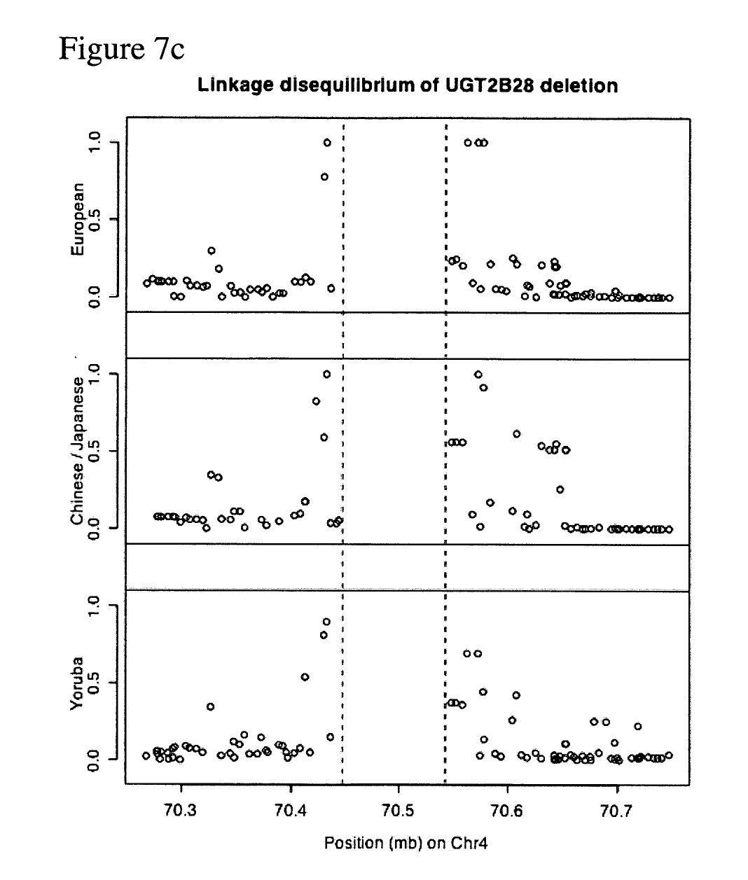

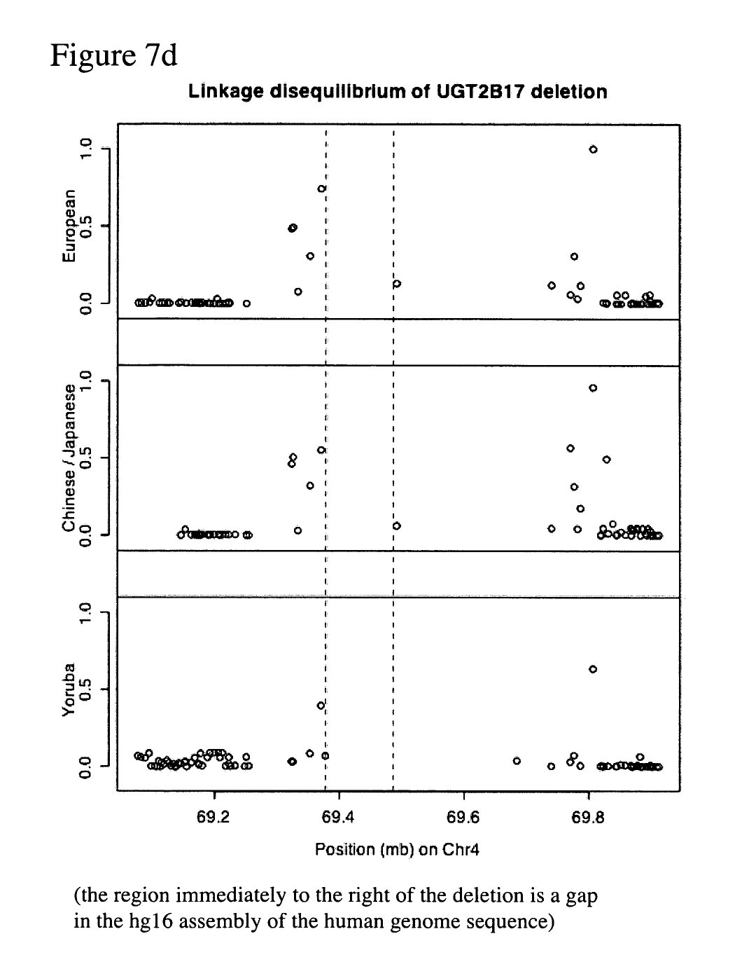

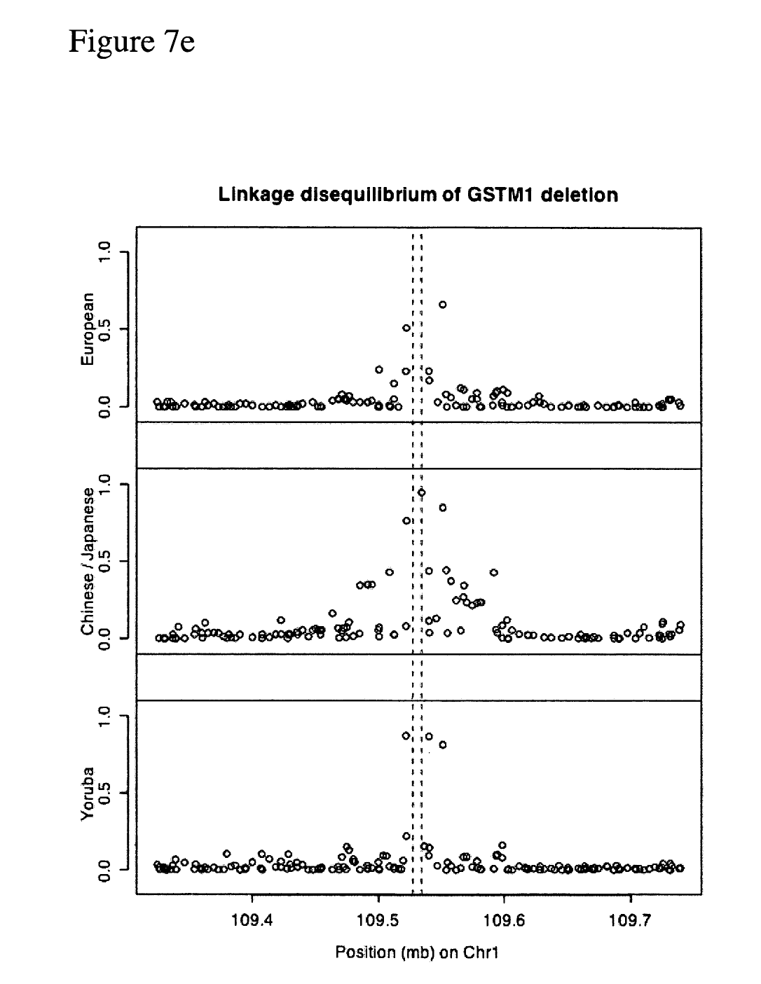

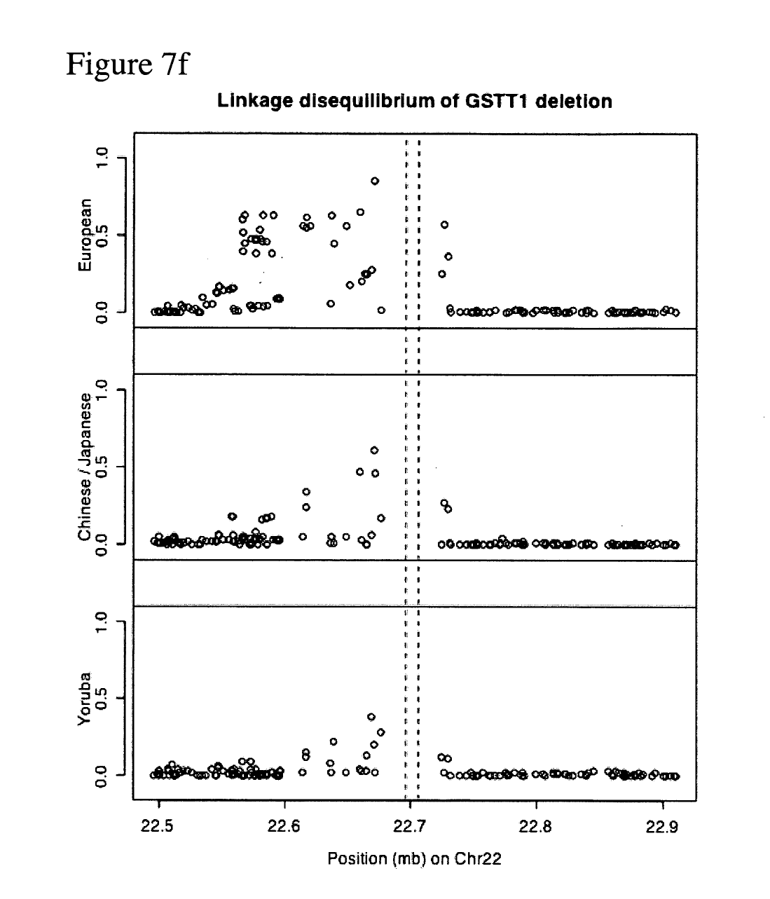

We observed strong LD between SNPs from HapMap and validated deletions. For example, nine of the ten gene deletions (for which we had designed accurate quantitative PCR genotyping assays) showed significant LD with nearby SNPs, and six of the ten had a perfect SNP proxy (r²=1) in one or more populations (see, for example FIG. 5A and FIGS. 7A-7F). In each case the deletion was associated to the same SNP allele(s) in each population (FIG. 5B and Table 3), indicating an ancestral mutation that occurred before humans migrated from Africa to Europe and Asia. In the larger collection of 51 deletion variants validated by PCR, we found elevated homozygosity at SNPs flanking the homozygous deletions (relative to randomly-selected individuals at the same loci), indicating that the deletion alleles travel on specific SNP haplotypes (FIG. 5C). On average, the rate of decay of haplotype homozygosity around deletion alleles was similar to that observed for a frequency- and population-matched set of SNP alleles (FIG. 5C).

TABLE 3
SNP alleles that tag common gene deletion alleles, for potential use
in medical genetic studies.

| | | | Tagging SNP allele (R²) | | |
|---|---|---|---|---|---|
| Gene | | | | Chinese/ | |
| deletion | SNP | SNP position | European | Japanese | Yoruba |
| UGT2B28 | rs4590108 | 70,430,000 | C(0.78) | | C(0.81) |
| | rs11249532 | 70,432,487 | T(1.0) | T(1.0) | T(0.90) |
| | rs12501393 | 70,562,708 | G(1.0) | | G(0.69) |
| | rs12501953 | 70,572,663 | C(1.0) | C(1.0) | C(0.69) |
| | rs12507041 | 70,577,410 | G(1.0) | G(0.91) | |
| UGT2B17 | rs2708666 | 69,370,214 | A(0.74) | A(0.55) | A(0.40) |
| | rs3100645 | 69,806,739 | C(1.0) | C(0.96) | C(0.63) |
| LCE3C | rs4112788 | 149,767,858 | C(0.93) | C(0.90) | |
| | rs1886734 | 149,807,724 | G(0.93) | G(0.93) | G(1.0) |
| | rs6700158 | 149,809,410 | G(0.93) | G(0.92) | G(1.0) |
| | rs4845459 | 149,820,424 | A(0.85) | A(0.93) | |
| TRY6 | rs13230029 | 141,907,602 | G(1.0) | G(0.97) | |
| | rs4726581 | 141,912,822 | C(1.0) | C(0.94) | |
| | rs4726582 | 141,912,983 | T(1.0) | T(0.97) | |
| | rs4726583 | 141,912,987 | T(1.0) | T(0.97) | |
| | rs2734212 | 141,936,451 | G(1.0) | G(0.97) | |
| | rs2734213 | 141,936,908 | C(1.0) | C(0.97) | |
| | rs2855983 | 141,939,191 | A(0.97) | A(0.97) | A(1.0) |
| | rs2734218 | 141,946,713 | C(0.97) | C(0.94) | C(0.92) |
| GSTM1 | rs2071487 | 109,531,808 | | C(1.0) | |
| | rs448934 | 109,534,259 | | A(1.0) | |
| | rs1858749 | 109,540,179 | | | G(0.97) |
| | rs366631 | 109,551,199 | T(0.76) | T(0.85) | T(0.91) |
| GSTT1 | rs5760147 | 22,659,502 | C(0.80) | C(0.83) | |
| | rs407257 | 22,671,104 | G(1.0) | G(1.0) | G(0.48) |

Our results indicate that the human genome has hundreds of common, multi-kilobase deletion variants, including some that remove genes, and that SNPs can be used to discover, analyze, and serve as markers for these variants. While we have used this approach on the HapMap, the same approach can be used to search for deletion variants in any set of SNP genotypes, such as data from imminent whole-genome association studies. Discarded, “failed” assays from earlier medical genetics studies could also be re-examined to search for the spatially patterned signature of a segregating deletion. Such an approach could be used together with intensity data from genotyping assays (FIG. 3A and Zhao et al., Cancer Res. 64:3060-3071 (2004)) to routinely identify deletions in genetic studies.

We describe an initial catalog of common deletion variants, but it is just a first draft toward a complete catalog. We have detected only those deletions large enough to affect multiple, independent HapMap SNP assays; most deletions smaller than 5 kb would not be detected at the current HapMap marker density. Phase 2 of the HapMap, with an assay every 1 kb, will considerably increase this resolution. The low density of HapMap assays in very-recently-duplicated regions of the genome has also impeded our discovery of deletions there; thus, our findings are limited to deletions of relatively unique sequences. Other types of structural variants, such as multi-copy duplications, may be more susceptible to recurrent structural mutation and therefore show less linkage disequilibrium. The application of diverse methods for finding structural variants (Sebat et al., supra; Iafrate et al., supra; Tuzun et al., supra; Sharp et al., supra; and Fredman et al., Nat. Genet. 36:861-866 (2004)), together with the development of follow-on genotyping assays, will allow more-complete catalogs of structural variants and their linkage disequilibrium properties.

Most importantly, an integrated view of structural variation and SNP variation is critical to medical genetics. To the extent that common deletion variants are in linkage disequilibrium, their association to disease can be discovered by the kinds of strategies proposed for SNP association studies (HapMap consortium, Altshuler et al. Nature 437:1299-1320 (2005)). In the future, medical genetics will benefit from a full catalog of common variants, since all types of alleles must be considered in an unbiased search for the causes of disease.

Materials and Methods

Fluorescent in situ Hybridization (FISH)

Fosmid clones with end sequences mapped to locations within predicted deletion intervals were obtained from the BAC/PAC resource, and DNA was isolated from each fosmid with the Maxi DNA plasmid kit (Qiagen). Fosmid DNAs were then labeled by nick translation with Spectrum Green-11-dUTP (G248P89259F2 and G248P87989C3 on chromosome 4) or Spectrum Orange-11-dUTP [Vysis, Inc.] (G248P87609A7 on chromosome 8 and G248P81036F4 on chromosome 18). We co-hybridized the test probes with appropriate positive control probes: Spectrum Orange-11-dUTP-labeled BAC clone RP11-363G1 (BAC/PAC; chromosome 4p15.1), and biotin-16-dUTP-labeled chromosome 8 and 18 paint probes (Roche). FISH experiments were performed using standard hybridization conditions on metaphase chromosome preparations derived from lymphoblastoid cell lines obtained from the Coriell Institute for Medical Research. Cy5-labeled streptavidin was used for detection of the biotin labeled chromosome 8 and 18 paint probes. Images were captured on an Olympus AX70 fluorescent microscope equipped with a CCD camera (Photometrics KAF 1400) with appropriate fluorescent filters and analyzed with Applied Imaging's Genus software.

The chromosome 4 fosmids used for FISH validation (G248P89259F2 and G248P87989C3) are mapped to segmental duplication-containing regions (Sebat et al., supra). Sequences with >94% nucleotide similarity are located <1 Mb (on chromosome 4) from each fosmid (http://genome.ucsc.edu). We considered the possibility that these probes could hybridize to a segmental duplication and yield a positive FISH signal, even if the target sequence were deleted. To investigate this, we repeated these experiments six times under various hybridization conditions, including once with an extended hybridization of 48 hours. In four out of these six experiments for a given probe and in a minimum of 25 metaphase spreads examined per individual, we consistently observed zero fluorescent probe signals (e.g., for fosmid probe G248P89259F2: NA19098), one signal (NA19100, NA19200, NA19202), or two fluorescent probe signals (NA19099, NA19201) per individual. Furthermore, in these experiments we included parent-offspring trios and FISH results were consistent with Mendelian inheritance of deletions. In two experiments (including the 48 hour hybridization protocol), those individuals believed to be homozygous for the deletion, heterozygous for the deletion, and homozygous for the non-deletion allele were observed in a minimum of 25 metaphase spreads per individual to have two faint signals (e.g., for fosmid probe G248P89259F2: NA19098), one faint and one strong signal (NA19100, NA19200, NA19202), and two strong signals (NA19099, NA19201), respectively. FIG. 6C shows such a signal intensity difference in an individual heterozygous for the chromosome 4 deletion containing fosmid G248P87989C3.

PCR Validation of Homozygous Deletion Variants

To validated predicted homozygous deletions by PCR, we selected 60 candidate deletion loci for which the pattern of genotypes predicted the existence of at least two individuals with homozygous deletions in at least one population. The criterion for validation was confirmation of a precise predicted pattern of amplification success and amplification failure across at least 12 samples that included at least two predicted examples of each result. Any deviation from that pattern was classified as a confirmation failure. The predictions (about which individuals harbored homozygous deletions) were derived from the SNP genotypes—the individuals in whom multiple null genotypes had given rise to the predicted deletion variant (Appendix A) were predicted to be homozygous null; all other individuals were predicted to have genetic material at that locus. Importantly, we chose PCR amplification sites that were distinct from any of the sequences used in the SNP genotyping assays, so that this would be an independent confirmation of a predicted result. Table 4 includes a list of PCR primers that were used in PCR assays for each deletion variant.

TABLE 4

PCR assays for homozygous deletions.

Deletion variant

| | | | |
|---|---|---|---|
| SEQ | Left | Right | PCR primers used |

| | | | | |
|---|---|---|---|---|
| ID | supporting | supporting | Forward primer | Reverse primer |

| | | | | | |
|---|---|---|---|---|---|
| NO | Chr | marker | marker | sequence | sequence |

| | | | | | |
|---|---|---|---|---|---|
| 19 | chr2 | 11,042,732 | 11,043,694 | TAGAGGCAGGGGC | CTAAATGCCATATT |
| | | | | TCTAAAAGTTC | TTTGTATGGAGGGC |
| 20 | chr2 | 89,093,935 | 89,175,498 | TGTGGTTGCAAGG | TTCCTCTTCCCGTG |
| | | | | CTGATTTCC | AACTCTAATTTTC |
| 21 | chr2 | 123,575,969 | 123,577,446 | AGTGGCATGGCAG | CCAATCATTTTACA |
| | | | | CAGGG | TTTGTAAACTCAAC |
| | | | | | TACGTC |
| 22 | chr2 | 129,734,701 | 129,735,219 | CACCTTCCAGATG | AACAATGCATCACT |
| | | | | GGAATGCAC | ACCACCACCC |
| 23 | chr2 | 238,086,689 | 238,093,885 | GCTGAATAATAGC | CGCTCCATATCAAA |
| | | | | GTAAGGGACAGGC | GAGTAAGCAATC |
| 24 | chr3 | 99,731,950 | 99,732,552 | CTCATGGCACCCA | CACCTATTTGAGGA |
| | | | | TATAAAAAGCTCC | TACAGACCAAACCT |
| | | | | | ACC |
| 25 | chr3 | 133,032,620 | 133,033,926 | AAACACCCTGATT | CTTTCTAGAGCTCA |
| | | | | TCGTGTGCAGG | TCTTCTACCAAGGG |
| | | | | | C |
| 26 | chr3 | 133,312,348 | 133,312,893 | GATTTGAGGAACC | TTACACAACAGAGC |
| | | | | TCCTTTGAGAGAC | CTTTCTCCGGC |
| | | | | AC | |
| 27 | chr3 | 163,538,907 | 163,550,497 | GAGTGATTTGTAT | AGTTCTTTCTAATA |
| | | | | CATATAGGCAGCC | AATAACCAATAAAT |
| | | | | CTTC | CAAACC |
| 28 | chr3 | 190,685,007 | 190,688,562 | TTACAACTGGTTA | AAATATGGGGCCAT |
| | | | | CTCCAGTAGAAGT | GGCATTCG |
| | | | | ATGTAGGC | |
| 29 | chr3 | 192,388,157 | 192,390,897 | AGCAGAGTTAGTT | CCACATCCCCCACA |
| | | | | TAGCAAAATGTCC | TACACACATAC |
| | | | | CC | |
| 30 | chr4 | 9,969,524 | 9,980,122 | GGTGATTATACTT | AGACCACTTGCCAA |
| | | | | TCCACCTGCTCAT | AACTCCCAG |
| | | | | C | |
| 31 | chr4 | 21,123,929 | 21,126,700 | ACAGGTAGGTGGG | TCTGCCCAATAATC |
| | | | | TAAATATGAAACT | TAGTAGGCATCCAG |
| | | | | TAGCTG | TC |
| 32 | chr4 | 34,677,422 | 34,724,191 | GTGTGCTCGGAGA | AACCATATAACCAC |
| | | | | AGAGCTCTAATTG | TGGGCCAACC |
| 33 | chr4 | 64,701,164 | 64,713,008 | ATGGAAAGCAGCA | AAAACACAAAATAT |
| | | | | GCCAAACTC | ACAATTATAATACC |
| | | | | | CCCTCC |
| 34 | chr4 | 115,637,062 | 115,641,110 | CATAAGGGCTGCC | TCCCACTGCATCAC |
| | | | | ATGTGCG | CCCAG |
| 35 | chr4 | 138,551,715 | 138,556,685 | TTCAACAGAAATG | ATGCATTCTAGAAC |
| | | | | TAATTGCTTACAT | AATGAACATGGGCT |
| | | | | GAG | G |
| 36 | chr4 | 152,458,392 | 152,461,345 | CTGAGTTATGTAT | AATATGGGGTGAGG |
| | | | | AGCCACTTTGGCC | ATAGAGGGGTGTG |
| | | | | G | |
| 37 | chr4 | 189,917,097 | 189,929,184 | AAGGGTTCAATGT | GAAGCCCGTCCAGT |
| | | | | TCAAAGGCATC | CCTGC |
| 38 | chr6 | 19,151,529 | 19,155,942 | GCAAAGGATCTTT | TTTATCATAAAAAG |
| | | | | ACTGAGCCAACC | TGTAGGGCAAGTAG |
| | | | | | ATG |
| 39 | chr7 | 89,422,556 | 89,424,327 | CATTGTTCTTAAT | CAGATGTGACAAAG |
| | | | | ATTGTTATCAAGG | TCTGGAAGG |
| | | | | GTCTC | |
| 40 | chr7 | 97,008,440 | 97,012,729 | CCTAAATCTTAAA | TGCCAACAAGCTGA |
| | | | | TAAGTTTGGTAGG | GTCCAAAGAC |
| | | | | AAAAAGGG | |
| 41 | chr7 | 125,601,054 | 125,603,762 | TTGGAATGGCTTA | ATTTTCTTAAATAA |
| | | | | CATCTTCTGTG | CCTTTAAGCGTGTG |
| | | | | | TC |
| 42 | chr7 | 141,188,320 | 141,199,669 | GTAAAGGGTCTTT | AACACACATGCCCT |
| | | | | GTAAATTTGGGTG | AAGTGTTTCTCC |
| | | | | G | |
| 43 | chr7 | 141,456,537 | 141,472,285 | GGCTGGATGACTA | CACACACACTACAT |
| | | | | GAGCCTACATC | ACTTGGATATATAC |
| | | | | | GTTTC |
| 44 | chr7 | 141,921,685 | 141,931,471 | ACAGGTGATAAAA | GCCCTCAGATGATT |
| | | | | GCCCGAGCC | ATTGGTGAAGTG |
| 45 | chr8 | 2,242,110 | 2,250,519 | CAACAAGCATGCA | AACCAGGGCAAGAC |
| | | | | AACATCAGTGTC | CCGC |
| 46 | chr8 | 39,250,107 | 39,404,547 | AGAAGAATATTTA | TTTGCAAGTTATGA |
| | | | | AACTCCTTATTGT | ATGCTGAGATG |
| | | | | GGCATAC | |
| 47 | chr8 | 54,202,942 | 54,211,318 | AAAGTTCCCCCTT | TTGATCCTTGGTCT |
| | | | | TGGGGAGG | TAGTTTAGCCTATT |
| | | | | | CC |
| 48 | chr8 | 55,414,544 | 55,423,847 | AAAGAAAATCTGT | TTTGTGTTGGTTTT |
| | | | | TCTGGAGATTCGT | TGAGGGCAAC |
| | | | | ATATTG | |
| 49 | chr8 | 59,355,794 | 59,368,409 | GAGTGCTTAAGAT | CACATCCCACCCAG |
| | | | | CCTGATATGAGTG | GCAGTTC |
| | | | | GC | |
| 50 | chr8 | 65,165,838 | 65,167,766 | AACCTCTGCCCCA | TACCTTCTCAGCAA |
| | | | | ACCCACC | GGCCATC |
| 51 | chr8 | 103,010,682 | 103,011,802 | CCTTGGGAAAGGT | GCATCCACAAAGTC |
| | | | | TTAGGGAGCG | TTCCAAACTGTAGG |
| 52 | chr8 | 115,591,865 | 115,599,078 | TGCACTGCAATGC | GATAGGATCTGATC |
| | | | | CACCTTTG | AGAAATGCACTCAG |
| | | | | | C |
| 53 | chr9 | 4,006,814 | 4,009,224 | TCCCTACCAGCCA | CAATCCAAGATTCG |
| | | | | AGGGGG | AAAATGCACG |
| 54 | chr9 | 32,991,449 | 33,014,917 | AATGCCTTTCCCA | CATGAGCAGCTTCC |
| | | | | GTTTCCTGTCC | TACCTGCC |
| 55 | chr9 | 124,896,840 | 124,898,353 | AGGAGAGGGTCAA | TGTTGCGTCACAGA |
| | | | | CCACCATAAGC | GGGGGAG |
| 56 | chr9 | 133,660,972 | 133,664,954 | CAGGCCCTGGAAG | CTGGTGCTTTCATT |
| | | | | TCCCC | GCGGAAAAC |
| 57 | chrX | 3,700,009 | 3,708,183 | GCAAATATCTAAT | TTTAACTACGATTA |
| | | | | TTTTCAAGATCCA | ATGATGGAATAGAG |
| | | | | GCCC | AAAGAC |
| 58 | chrX | 15,834,905 | 15,839,616 | AAAAACAAATAAA | GGTATGAAGTTGTG |
| | | | | CAATGGTGCCC | AAGTAGTGCCAG |
| 59 | chrX | 83,958,997 | 83,971,986 | CAACCTTCAAACT | CAGAGGAGTGTGTG |
| | | | | TTTGGGATAGAAG | ACTGCCCC |
| | | | | C | |
| 60 | chrX | 107,659,218 | 107,737,812 | GTTCCAAGAGATT | AGATTTTAAGGAAG |
| | | | | AAACCGATAAGAA | CACTCCACCCC |
| | | | | TACCTG | |
| 61 | chrX | 114,373,214 | 114,373,881 | CAGTAGGACAATG | ATCCTGGCCTTAGG |
| | | | | ACAAAGTAGGCTT | ATGAGGACG |
| | | | | AGCAG | |
| 62 | chrX | 140,165,208 | 140,166,897 | GACCACAGCACAT | AGCATAGCGACAAC |
| | | | | TCAACACATCC | CTATGCCTTCTG |
| 63 | chr12 | 27,539,977 | 27,545,038 | CCATGGCATTTTA | TGTTCACCTTCAAT |
| | | | | TGGCTCCC | ATCTCTCCACTCTT |
| | | | | | G |
| 64 | chr12 | 39,104,527 | 39,106,948 | AGAAAACAGCTAT | TCTTAGTCGGAGTC |
| | | | | ATGGTGGTTGAAC | ATGGGTATTTGC |
| | | | | TATG | |
| 65 | chr12 | 117,847,706 | 117,853,165 | GGTTTAAACTATT | GCTGTGCATGGCTA |
| | | | | TCCAGGTGGAAGA | GATGACTTGTACTC |
| | | | | GGC | |
| 66 | chr14 | 33,107,807 | 33,110,015 | ATTCCCACCAGCT | AGGTCAGAGAAGTG |
| | | | | CCCTTCACCAG | ACTGAAAAATGTGG |
| 67 | chr14 | 68,010,231 | 68,011,603 | TTTCTCCCTTCCT | GGCCTTGGCCATAG |
| | | | | CCAAACACGC | TCAACAAG |
| 68 | chr18 | 1,907,900 | 1,922,838 | GACCCCAAGGAGA | ATGTTTTCATAAAT |
| | | | | TTGCTGACC | TAACCCTAACCCAG |
| 69 | chr18 | 36,512,513 | 36,518,187 | CCTGGCTTCCTTA | AAAAGGCCTAACTA |
| | | | | TCAAACTCACTTA | GTGAACCCATGTAA |
| | | | | AC | TC |
| 70 | chr18 | 45,353,989 | 45,369,896 | GATCGAGCAGAAT | ACTTCACCCGGATG |
| | | | | GCCACCAAC | CGCC |
| 71 | chr18 | 46,252,952 | 46,257,058 | TCTGTAGAGTGTG | AAACCTCTGGCTCA |
| | | | | CCAGTTAGTACAG | GTGTTGG |
| | | | | CC | |
| 72 | chr18 | 64,895,177 | 64,904,477 | TTCTTCTCCTATA | GCTAAATAGTGGCA |
| | | | | TACCTGCTAATGC | AAGACCTCGTCTAC |
| | | | | TCAGTTC | C |
| 73 | chr22 | 47,865,647 | 47,867,222 | TCTCTATGCCAAT | GGACTCTCCTGATT |
| | | | | AAACAAATTGAAA | TTCAATCCAACC |
| | | | | TTAAC | |

Illumina (Two-Color, Allele-Specific Fluorescence) Validation of Deletion Variants

Seventeen candidate deletion variants covered at least three SNPs that had been assayed on the Illumina platform at the Broad Institute. The Illumina platform generates a quantitative allele-specific intensity measurement for each allele in each individual in a population. The normalized allele-specific intensity measurements are comparable across individuals and generally fall into two or three discrete clusters, corresponding to individuals homozygous for allele 1, individuals homozygous for allele 2, and individuals heterozygous for alleles 1 and 2. For SNPs covered by predicted deletion variants, we observed additional genotype classes corresponding to individuals hemizygous for allele 1, individuals hemizygous for allele 2, and individuals homozygous for the deletion allele. We considered a deletion variant validated if (i) we observed one or more of these additional, well-separated genotype clusters, and (ii) all of the individuals predicted (from multi-marker genotype patterns) to be hemizygous or homozygous deleted in fact fell into the appropriate additional cluster.

Quantitative PCR

Individuals' deletion genotypes cannot be unambiguously inferred from SNP genotypes data (see, for example, FIG. 2B). Therefore it was necessary to develop assays for accurately typing the deletion variants. We performed two-color TaqMan assays, using a FAM-labeled probe for the test gene and a HEX-labeled probe for PMP22, a euploid control gene. TaqMan amplification reagents were purchased from Applied Biosystems together with the following assays.

| | | |
|---|---|---|
| PMP22 | | |
| (control) | | |
| primer1 | CCCTTCTCAGCGGTGTCATC | (SEQ ID NO: 1) |
| primer2 | ACAGACCGTCTGGGCGC | (SEQ ID NO: 2) |
| probe | VIC-TTCGCGTTTCCGCAAGAT- | (SEQ ID NO: 3) |
| | MGBNFQ | |
| GSTM1 | | |
| primer1 | CTGTGTCCACCTGCATTCG | (SEQ ID NO: 4) |
| primer2 | GAGACCGGGCACTCACTGT | (SEQ ID NO: 5) |
| probe | 6FAM-TCAGTCCTGCCATGAGCAG | (SEQ ID NO: 6) |
| | GC-BHQ1 | |
| GSTT1 | | |
| primer1 | GGGATGGAAAGTCACGTCCT | (SEQ ID NO: 7) |
| primer2 | AGAGACTGGGACAGCGTCAA | (SEQ ID NO: 8) |
| probe | 6FAM-CAGAATCTCAGCAGCTGGG | (SEQ ID NO: 9) |
| | CCA-BHQ1 | |
| CYP2A6 | | |
| primer1 | AGGATGGGGACTTTTCCTTT | (SEQ ID NO: 10) |
| primer2 | TCCTCATCTTCAGCTGTTGG | (SEQ ID NO: 11) |
| probe | 6FAM-CATTCAGGATTCTGGGCTT | (SEQ ID NO: 12) |
| | GCTCC-BHQ1 | |
| OR51A2 | | |
| primer1 | TGCCAATTGCCTACTGTTTG | (SEQ ID NO: 13) |
| primer2 | AGCAACAGTGGAAGGAGAGAA | (SEQ ID NO: 14) |
| probe | 6FAM-GACAACATAACCAAGTGGG | (SEQ ID NO: 15) |
| | GCTTATTTTC-BHQ1 | |
| PRB1 | | |
| primer1 | TGAAGGGACCTCAGTAGTTGG | (SEQ ID NO: 16) |
| primer2 | TGACAGGCATGGTTCTTCTG | (SEQ ID NO: 17) |
| probe | 6FAM-CTGACTTTCTAGCAAGG-M | (SEQ ID NO: 18) |
| | GBNFQ | |
| UGT2B17 | Applied Biosystems | |
| | Hs00854486_sH | |
| UGT2B28 | Applied Biosystems | |
| | Hs00852540_s1 | |
| LCE3C | Applied Biosystems | |
| | Hs00708773_s1 | |

Small (60-90 nt) amplicons from the test and control loci were simultaneously amplified in the same tube, in 96-well plates (one plate per population, including five replicate samples and one blank sample) on a Bio-Rad iCycler. The threshold cycle (Ct) was calculated for each fluorophore separately, and the difference between the threshold cycles for the two fluorophores (delta_Ct) was used as a measurement of relative copy number that could be compared from sample to sample on the same plate. For each assay, the delta_Ct measurements clustered into three discrete groups (with one group typically showing no amplification of the test locus at all). For some assays, these groups were initially incompletely separated; in these cases, averaging of the delta_Ct measurements across 3-5 replicates resulted in discrete, well-separated clusters of average measurements. For each assay, we treated these three clusters as “+/+,” “+/−,” and “−/−” genotypes. In each case, the resulting genotype calls for replicate samples agreed completely, and the resulting genotypes showed Mendelian inheritance and Hardy-Weinberg equilibrium.

### Example 2

### Use of Common Deletion Variants for Determining Immunocompatibility in Bone Marrow Transplant

The non-MHC factors which determine histocompatibility are generally unknown. As a consequence, allogeneic transplantations carry risk due to unforeseen incompatibilities between donor and host. The human genome has recently been shown to exhibit large-scale deletion polymorphism, including many large common deletion variants that appear as homozygous deletions in a significant fraction of the population. In the following example of the methods of the invention we investigated whether deletion mismatches for common deletion variants (homozygous deletion in donor but not in host) were associated with graft-versus-host disease (GVHD) following allogeneic hematopoetic stem cell transplantation (aHSCT).

Using the methods described below, we evaluated 500 aHSCT cases involving HLA-identical sibling donor-recipient pairs. We typed donors and patients for the presence of six gene deletions, and assessed whether aGVHD and cGVHD occurrence were associated with mismatch for these gene deletions. We found that mismatch for two common deletion variants, UGT2B28 and UGT2B17, was associated with chronic GVHD, and, for UGT2B17, was also associated with acute GVHD. These results demonstrate that large deletion variants may contribute to histoincompatibilities among individuals, and validate the usefulness of the invention described in this application. GVHD risk might be reduced by prospectively typing donors and patients for deletion variants in UGT2B17 and UGT2B28 genes.

Patients

The main study population consisted of 500 aHSCT recipients and their HLA-identical sibling donors. Inclusion criteria were the use of full myeloablative aHSCT. All recipients and donors gave written informed consent according to protocols approved by the institutional review boards of Helsinki University Central Hospital and the Dana Farber Cancer Institute (protocol 01-206).

The aGVHD replication study population consisted of 336 aHSCT recipients and their HLA-identical sibling donors, collected as described previously (Nichols et al., Blood. Dec. 15, 1996;88(12):4429-34).

Genotyping of Deletion Variants

We developed a quantitative PCR assay for typing each deletion variant in each donor and patient. In this assay, the locus of interest and a control, two-copy locus (PMP22) are simultaneously amplified in a 20 μl reaction containing TaqMan Master Mix (Applied Biosystems) together with a forward primer, a reverse primer, and a dual-labeled probe for each locus. The probe for the test locus (gene deletion polymorphism) is labeled with FAM and a BHQ-1 quencher (IDT); the probe for the control locus is labeled with VIC and an MGB quencher (Applied Biosystems). The simultaneous amplification of the test and control loci is monitored by real-time PCR and a threshold cycle (Ct) is determined separately for each locus by separation of the FAM and VIC spectra.

A sample was determined to be homozygous deleted for the test locus if the control locus showed robust amplification (Ct<32) while the test locus failed to amplify after 40 cycles. The quantity δCt =Ct_{—control}−Ct_{gene} showed a discrete, bimodal distribution across the remaining, non-homozygous deleted samples; samples from the higher δCt cluster were determined to have two copies of the gene, and samples from the lower δCt cluster were determined to have one copy. As a quality-control check, we verified that both of the following were true: (i) membership in the three genotype classes (corresponding to 0, 1, 2 copies) showed Hardy-Weinberg equilibrium and (ii) sibling genotypes were correlated across the cohort: regression of patient genotypes against the genotypes of their sibling donors yielded a regression coefficient that was not significantly different from 0.5.

Determination of Mismatches

Transplants were determined to involve a donor-recipient “deletion mismatch” for a deletion variant if the donor was homozygous deleted for that gene, and the recipient had a positive number (1 or 2) of gene copies. Transplants were considered to involve a “sex mismatch” if they involved a female donor and a male recipient.

Statistical Analysis

Acute and chronic GVHD were diagnosed and graded according to standard criteria. Acute GVHD cases were those with grades 2-4 aGVHD; controls were those with grades 0-1 GVHD. Chronic GVHD cases were those with “limited” or “extensive” cGVHD; cGVHD controls were those with “no” cGVHD.

The relationship between deletion variant mismatches and GVHD status was first assessed by association analysis of mismatch at each individual locus with aGVHD and cGVHD, using the 360 donor-recipient pairs who had no known mismatch risk factors (no sex mismatch). We performed a one-sided chi-square test.

The Michigan aGVHD cohort was used for replication analysis of the single locus showing positive association for aGVHD in the initial analysis, assessed by association analysis of mismatch at each individual locus with aGVHD. We performed a one-sided chi-square test.

Two loci (UGT2B17 and UGT2B28) were found to show positive associations in the initial analysis and were then assessed in the full cohort of 836 donor-recipient pairs using a regression model for GVHD risk whose terms included age, transplantation year, sex mismatch, UGT2B17 mismatch, UGT2B28 mismatch, and the interaction terms sex+UGT2B17 mismatch and sex+UGT2B28 mismatch.

These results demonstrate that deletion variants may contribute to histoincompatibilities among individuals. GVHD risk might be reduced by prospectively typing donors and patients for UGT2B17 and UGT2B28 gene deletions.

### Example 3

### Use of Deletion Variants for Determining Immunocompatibility in Organ Transplant

As described for Example 2, the non-MHC factors which determine histocompatibility are generally unknown. As a consequence, allogeneic organ transplantations carry risk due to unforeseen incompatibilities between donor and host. This study was designed to investigate whether mismatch for common deletion variants (homozygous deletion in donor but not in host) is associated with host-versus-graft disease (HVGD) following kidney transplantation.

Patients

The first study population consists of 500 renal allograft recipients and their HLA-identical sibling donors. The second study population consists of 700 renal allograft recipients and their unrelated donors. All recipients and sibling donors provided written informed consent according to protocols approved by the institutional review boards of Massachusetts General Hospital, Helsinki University Central Hospital, and Hospital do Rim o Hipertensao Sao Paulo.

Genotyping of Deletion Variants

Samples from each of the patient populations have been collected and will be used for the genotyping of deletion variants as described in Example 2. Methods for analysis of the samples after genotyping of deletion variants is performed are described below.

Determination of Deletion Variant Mismatches

Transplants are determined to involve a donor-recipient “mismatch” for a deletion variant if the recipient had a deletion variant in all copies of the gene (i.e., homozygous deletion) and the donor had a positive number (1 or 2) of gene copies.

Statistical Analysis

Renal allograft rejection was diagnosed and graded according to standard criteria. The primary diagnostic categories used in this study are “rejection,” “no rejection,” and “days to rejection.”

For the first (sibling-donor) study population, the relationship between gene deletion mismatches and rejection status is assessed by association analysis of mismatch at each individual locus with risk of rejection. A one-sided chi-square test is used to assess whether mismatch is associated with increased risk of rejection.

Any two loci found to show positive associations in the initial analysis are then assessed using a regression model for rejection risk whose terms include age, donor sex, recipient sex, transplantation year, cold ischemia time, and mismatch for each of the deletion variants.

For the second (unrelated-donor) study population, we assess the contribution of gene deletion mismatches using using a regression model for rejection risk whose terms include age, donor sex, recipient sex, transplantation year, cold ischemia time, mismatch for each of the gene deletion polymorphisms, and the numbers of HLA-AB and HLA-DR mismatches.

### Appendix A: Predicted Deletion Variants and Supporting SNP Evidence

This table lists 541 predicted deletion variants identified from patterns of SNP assay failures in the Phase 1 Hapmap, as described in this study. The three leftmost columns show the location of the predicted deletion variant (the genomic coordinates spanned by all SNPs that supported the prediction). The five rightmost columns describe the evidence supporting each prediction: the locations of SNP assays, the population and type of supporting evidence, and the individuals in whose genotypes that evidence was observed.

Key to populations:
CEU 90 individuals (30 trios) of European ancestry, sampled in Utah, USA
YRI 90 individuals (30 trios) of Yoruba ancestry, sampled in Ibadan, Nigeria
JCH 45 unrelated individuals of Han Chinese ancestry, sampled in Beijing, plus
   44 unrelated individuals of Japanese ancestry, sampled in Tokyo

All physical coordinates are shown on the hg16 build of the human genome.

| | Predicted deletion | | | | | | |
|---|---|---|---|---|---|---|---|
| | variant | | Supporting evidence | | | | |
| | Leftmost | Rightmost | Leftmost | Rightmost | | | |
| | supporting | supporting | supporting | supporting | | | |
| Chr | marker | marker | marker | marker | Pop | Type of evidence | Individuals |
| chr1 | 55,871 | 68,941 | 55,871 | 68,941 | CEU | Mendelian inconsistencies | (NA12155, NA10831), (NA12044, NA10857) |
| | | | 55,871 | 68,941 | YRI | Mendelian inconsistencies | (NA18912, NA18914), (NA19207, NA19208) |
| chr1 | 10,084,725 | 10,087,962 | 10,084,725 | 10,087,962 | YRI | Mendelian inconsistencies | (NA18502, NA18500), (NA18912, NA18914), |
| | | | | | | | (NA19130, NA19132) |
| chr1 | 10,415,637 | 10,427,143 | 10,415,637 | 10,427,143 | CEU | Mendelian inconsistencies | (NA12146, NA10847) |
| chr1 | 12,541,839 | 12,549,327 | 12,541,839 | 12,549,327 | YRI | Mendelian inconsistencies | (NA19101, NA19103) |
| chr1 | 16,392,736 | 16,405,381 | 16,395,820 | 16,405,381 | CEU | Mendelian inconsistencies | (NA12815, NA12802) |
| | | | 16,392,736 | 16,400,201 | YRI | Mendelian inconsistencies | (NA19138, NA19139) |
| chr1 | 16,634,909 | 16,646,447 | 16,634,909 | 16,646,447 | YRI | Mendelian inconsistencies | (NA19207, NA19208), (NA19238, NA19240) |
| chr1 | 34,606,761 | 34,610,715 | 34,606,761 | 34,610,715 | CEU | Mendelian inconsistencies | (NA12154, NA10830), (NA11839, NA10854), |
| | | | | | | | (NA12892, NA12878), (NA12763, NA12753) |
| chr1 | 43,330,703 | 43,346,983 | 43,330,703 | 43,346,983 | YRI | Mendelian inconsistencies | (NA18853, NA18854) |
| chr1 | 61,811,705 | 61,813,163 | 61,811,705 | 61,813,163 | YRI | Mendelian inconsistencies | (NA19203, NA19205), (NA19222, NA19221), |
| | | | | | | | (NA19098, NA19100) |
| chr1 | 72,137,668 | 72,176,870 | 72,142,473 | 72,161,849 | CEU | Mendelian inconsistencies | (NA11832, NA10855), (NA12154, NA10830), |
| | | | | | | | (NA07000, NA07029), (NA07022, NA07019), |
| | | | | | | | (NA12043, NA10857), (NA12873, NA12864), |
| | | | | | | | (NA12874, NA12865), (NA06985, NA06991), |
| | | | | | | | (NA12751, NA12740) |
| | | | 72,139,345 | 72,176,870 | CEU | Mendelian inconsistencies | (NA12003, NA10838), (NA12006, NA10839), |
| | | | | | | | (NA12057, NA10851), (NA11993, NA10860), |
| | | | | | | | (NA11994, NA10861), (NA12145, NA10846), |
| | | | | | | | (NA12716, NA12707), (NA12874, NA12865), |
| | | | | | | | (NA11881, NA10859) |
| | | | 72,137,668 | 72,147,489 | YRI | Mendelian inconsistencies | (NA18501, NA18500), (NA18508, NA18506), |
| | | | | | | | (NA18516, NA18515), (NA19200, NA19202), |
| | | | | | | | (NA19160, NA19161), (NA19143, NA19145) |
| | | | 72,139,345 | 72,176,870 | CEU | Hardy-Weinberg | population |
| | | | 72,137,668 | 72,178,467 | YRI | Hardy-Weinberg | population |
| | | | 72,139,345 | 72,178,467 | JCH | Hardy-Weinberg | population |
| chr1 | 85,825,062 | 85,827,089 | 85,825,062 | 85,827,089 | CEU | Mendelian inconsistencies | (NA12762, NA12753) |
| chr1 | 87,059,987 | 87,062,651 | 87,059,987 | 87,062,651 | CEU | Mendelian inconsistencies | (NA11831, NA10855) |
| chr1 | 87,784,378 | 87,792,857 | 87,784,378 | 87,792,857 | CEU | Null genotypes | NA10839 |
| chr1 | 91,257,677 | 91,268,008 | 91,257,677 | 91,268,008 | CEU | Mendelian inconsistencies | (NA07000, NA07029) |
| chr1 | 94,609,885 | 94,625,063 | 94,622,667 | 94,623,588 | YRI | Mendelian inconsistencies | (NA18505, NA18503), (NA19141, NA19142) |
| | | | 94,609,885 | 94,625,063 | YRI | Mendelian inconsistencies | (NA18505, NA18503) |
| chr1 | 102,291,054 | 102,291,746 | 102,291,054 | 102,291,746 | CEU | Mendelian inconsistencies | (NA11995, NA10861), (NA07055, NA07048) |
| chr1 | 109,527,309 | 109,534,259 | 109,527,309 | 109,534,259 | CEU | Null genotypes | NA12043, NA12264 |
| chr1 | 110,677,705 | 110,681,790 | 110,679,447 | 110,681,702 | CEU | Null genotypes | NA12264, NA12812, NA12801, NA12814 |
| | | | 110,677,705 | 110,681,790 | CEU | Mendelian inconsistencies | (NA11992, NA10860), (NA11882, NA10859), |
| | | | | | | | (NA12249, NA10835) |
| chr1 | 114,106,286 | 114,107,358 | 114,106,286 | 114,107,358 | CEU | Mendelian inconsistencies | (NA12044, NA10857), (NA12760, NA12752) |
| chr1 | 119,469,803 | 119,473,878 | 119,469,803 | 119,473,878 | YRI | Mendelian inconsistencies | (NA18501, NA18500) |
| chr1 | 142,902,233 | 142,921,305 | 142,902,233 | 142,921,305 | CEU | Mendelian inconsistencies | (NA12814, NA12802) |
| chr1 | 146,404,349 | 146,405,202 | 146,404,349 | 146,405,202 | CEU | Mendelian inconsistencies | (NA12044, NA10857) |
| chr1 | 146,563,653 | 146,580,520 | 146,569,781 | 146,580,520 | CEU | Mendelian inconsistencies | (NA12044, NA10857),(NA07055, NA07048) |
| | | | 146,563,653 | 146,572,086 | YRI | Mendelian inconsistencies | (NA18508, NA18506), (NA18856, NA18857) |
| chr1 | 146,591,613 | 146,605,848 | 146,591,613 | 146,605,848 | YRI | Mendelian inconsistencies | (NA19099, NA19100) |
| chr1 | 148,535,315 | 148,561,285 | 148,535,315 | 148,561,285 | YRI | Mendelian inconsistencies | (NA18853, NA18854) |
| chr1 | 149,296,571 | 149,300,621 | 149,296,571 | 149,300,621 | CEU | Mendelian inconsistencies | (NA12716, NA12707) |
| chr1 | 149,771,758 | 149,800,260 | 149,785,060 | 149,797,102 | YRI | Mendelian inconsistencies | (NA18505, NA18503), (NA18517, NA18515), |
| | | | | | | | (NA19128, NA19129), (NA19238, NA19240) |
| | | | 149,786,102 | 149,798,424 | YRI | Mendelian inconsistencies | (NA18505, NA18503), (NA18517, NA18515), |
| | | | | | | | (NA18912, NA18914), (NA19200, NA19202), |
| | | | | | | | (NA19207, NA19208), (NA19160, NA19161), |
| | | | | | | | (NA19143, NA19145), (NA19128, NA19129), |
| | | | | | | | (NA19238, NA19240) |
| | | | 149,786,102 | 149,800,260 | CEU | Mendelian inconsistencies | (NA12057, NA10851), (NA11829, NA10856), |
| | | | | | | | (NA12155, NA10831), (NA07056, NA07019), |
| | | | | | | | (NA12145, NA10846), (NA12717, NA12707), |
| | | | | | | | (NA12891, NA12878), (NA12760, NA12752), |
| | | | | | | | (NA12761, NA12752), (NA12763, NA12753), |
| | | | | | | | (NA07034, NA07048), (NA07055, NA07048), |
| | | | | | | | (NA06993, NA06991), (NA06985, NA06991), |
| | | | | | | | (NA11882, NA10859) |
| | | | 149,771,758 | 149,798,424 | YRI | Mendelian inconsistencies | (NA18502, NA18500), (NA18505, NA18503), |
| | | | | | | | (NA18517, NA18515), (NA19200, NA19202), |
| | | | | | | | (NA19207, NA19208), (NA19160, NA19161), |
| | | | | | | | (NA19119, NA19120), (NA19143, NA19145), |
| | | | | | | | (NA19128, NA19129), (NA19238, NA19240) |
| | | | 149,771,758 | 149,798,424 | YRI | Hardy-Weinberg | population |
| | | | 149,774,642 | 149,800,260 | JCH | Hardy-Weinberg | population |
| chr1 | 149,977,953 | 149,986,389 | 149,977,953 | 149,986,389 | CEU | Mendelian inconsistencies | (NA12057, NA10851), (NA11829, NA10856), |
| | | | | | | | (NA11832, NA10855), (NA07034, NA07048) |
| | | | 149,977,953 | 149,982,821 | YRI | Mendelian inconsistencies | (NA19210, NA19211) |
| | | | 149,977,953 | 149,986,389 | CEU | Hardy-Weinberg | population |
| chr1 | 155,706,737 | 155,707,243 | 155,706,737 | 155,707,243 | CEU | Mendelian inconsistencies | (NA12249, NA10835) |
| chr1 | 155,737,529 | 155,738,184 | 155,737,529 | 155,738,184 | YRI | Mendelian inconsistencies | (NA19102, NA19103), (NA19207, NA19208) |
| chr1 | 160,080,024 | 160,080,653 | 160,080,024 | 160,080,653 | CEU | Mendelian inconsistencies | (NA12751, NA12740) |
| | | | 160,080,024 | 160,080,653 | YRI | Mendelian inconsistencies | (NA18508, NA18506), (NA18871, NA18872), |
| | | | | | | | (NA19116, NA19120), (NA19127, NA19129), |
| | | | | | | | (NA19131, NA19132), (NA19238, NA19240) |
| chr1 | 172,037,243 | 172,041,015 | 172,037,243 | 172,041,015 | CEU | Mendelian inconsistencies | (NA12154, NA10830), (NA12144, NA10846), |
| | | | | | | | (NA12248, NA10835) |
| chr1 | 187,000,430 | 187,069,828 | 187,022,238 | 187,058,520 | CEU | Mendelian inconsistencies | (NA12892, NA12878) |
| | | | 187,000,430 | 187,069,828 | CEU | Mendelian inconsistencies | (NA12892, NA12878) |
| chr1 | 194,080,727 | 194,081,468 | 194,080,727 | 194,081,468 | YRI | Mendelian inconsistencies | (NA18502, NA18500), (NA18505, NA18503), |
| | | | | | | | (NA18523, NA18521), (NA18852, NA18854), |
| | | | | | | | (NA18862, NA18863), (NA19102, NA19103), |
| | | | | | | | (NA19159, NA19161), (NA19140, NA19142), |
| | | | | | | | (NA19152, NA19154) |
| chr1 | 206,205,376 | 206,209,672 | 206,205,376 | 206,209,672 | CEU | Mendelian inconsistencies | (NA12056, NA10851), (NA11995, NA10861), |
| | | | | | | | (NA12044, NA10857), (NA12264, NA10863), |
| | | | | | | | (NA12234, NA10863), (NA06993, NA06991), |
| | | | | | | | (NA12751, NA12740) |
| chr1 | 226,783,490 | 226,788,594 | 226,783,490 | 226,788,594 | CEU | Mendelian inconsistencies | (NA12004, NA10838) |
| chr1 | 240,005,624 | 240,011,152 | 240,005,624 | 240,011,152 | CEU | Mendelian inconsistencies | (NA11830, NA10856), (NA11993, NA10860), |
| | | | | | | | (NA07022, NA07019), (NA12762, NA12753), |
| | | | | | | | (NA11881, NA10859) |
| chr2 | 3,782,046 | 3,786,654 | 3,782,046 | 3,786,654 | CEU | Null genotypes | NA12873, NA12248 |
| chr2 | 11,042,732 | 11,043,694 | 11,042,732 | 11,043,694 | CEU | Null genotypes | NA07000, NA12813 |
| chr2 | 17,207,641 | 17,216,891 | 17,207,641 | 17,216,891 | CEU | Null genotypes | NA12812, NA12801 |
| | | | 17,210,418 | 17,212,022 | CEU | Mendelian inconsistencies | (NA12812, NA12801) |
| chr2 | 18,156,325 | 18,177,354 | 18,156,325 | 18,177,354 | YRI | Null genotypes | NA19094, NA19093, NA19206 |
| | | | 18,160,633 | 18,171,634 | YRI | Mendelian inconsistencies | (NA19093, NA19094) |
| chr2 | 18,374,137 | 18,374,728 | 18,374,137 | 18,374,728 | CEU | Mendelian inconsistencies | (NA11994, NA10861) |
| chr2 | 24,576,974 | 24,580,587 | 24,576,974 | 24,580,587 | YRI | Mendelian inconsistencies | (NA18507, NA18506), (NA18852, NA18854), |
| | | | | | | | (NA19204, NA19205) |
| chr2 | 29,386,658 | 29,401,473 | 29,386,658 | 29,401,473 | JCH | Null genotypes | NA18579 |
| chr2 | 29,611,575 | 29,629,488 | 29,611,575 | 29,629,488 | JCH | Null genotypes | NA18579 |
| chr2 | 30,137,687 | 30,155,192 | 30,137,687 | 30,155,192 | JCH | Null genotypes | NA18579 |
| chr2 | 30,230,147 | 30,242,577 | 30,230,147 | 30,242,577 | JCH | Null genotypes | NA18579 |
| chr2 | 30,381,437 | 30,392,829 | 30,381,437 | 30,392,829 | JCH | Null genotypes | NA18579 |
| chr2 | 34,688,262 | 34,696,094 | 34,688,262 | 34,696,094 | CEU | Null genotypes | NA12005, NA10855, NA11994, NA12155, |
| | | | | | | | NA07019, NA12716, NA12891, NA12812, |
| | | | | | | | NA12813, NA12801, NA12874, NA12865, |
| | | | | | | | NA06993, NA06985, NA06991, NA12751 |
| chr2 | 35,560,170 | 35,589,400 | 35,560,170 | 35,589,400 | YRI | Null genotypes | NA18501, NA18506, NA18507, NA18913, |
| | | | | | | | NA19221, NA19222, NA19240, NA19239 |
| chr2 | 41,213,645 | 41,221,389 | 41,213,645 | 41,220,036 | YRI | Null genotypes | NA18504, NA18862, NA19201, NA19130 |
| | | | 41,213,645 | 41,221,389 | JCH | Null genotypes | NA18612 |
| | | | 41,216,804 | 41,220,036 | YRI | Hardy-Weinberg | population |
| chr2 | 46,697,753 | 46,700,744 | 46,697,753 | 46,700,744 | CEU | Mendelian inconsistencies | (NA12146, NA10847) |
| chr2 | 52,726,065 | 52,757,084 | 52,726,065 | 52,756,887 | CEU | Null genotypes | NA10856, NA11993, NA07056, NA07019, |
| | | | | | | | NA12717, NA12891, NA12878, NA12812, |
| | | | | | | | NA12815, NA12762, NA12753, NA11881, |
| | | | | | | | NA10859, NA12249 |
| | | | 52,726,065 | 52,757,084 | JCH | Null genotypes | NA18524, NA18547, NA18609, NA18608, |
| | | | | | | | NA18564, NA18545, NA18542, NA18561, |
| | | | | | | | NA18537, NA18579, NA18570, NA18571, |
| | | | | | | | NA18620, NA18621, NA18637, NA18526, |
| | | | | | | | NA18953, NA18968, NA18964, NA18940, |
| | | | | | | | NA18951, NA18947, NA18949, NA18948, |
| | | | | | | | NA18952, NA18975, NA18991, NA18994, |
| | | | | | | | NA18992, NA19007, NA18990, NA18976, |
| | | | | | | | NA18978, NA18995, NA18981 |
| | | | 52,726,065 | 52,757,084 | JCH | Hardy-Weinberg | population |
| chr2 | 54,667,738 | 54,688,600 | 54,667,738 | 54,688,600 | YRI | Null genotypes | NA19192 |
| chr2 | 55,301,625 | 55,307,769 | 55,301,625 | 55,307,769 | YRI | Null genotypes | NA19142, NA19141 |
| chr2 | 57,382,183 | 57,389,886 | 57,382,183 | 57,389,886 | CEU | Null genotypes | NA12155, NA10831 |
| chr2 | 59,598,122 | 59,600,156 | 59,598,122 | 59,600,156 | JCH | Null genotypes | NA18542, NA18561, NA18621 |
| chr2 | 71,306,841 | 71,317,129 | 71,306,841 | 71,317,129 | YRI | Null genotypes | NA18505, NA18857, NA18855, NA18913, |
| | | | | | | | NA19139, NA19138 |
| chr2 | 75,341,474 | 75,346,125 | 75,341,474 | 75,346,125 | CEU | Null genotypes | NA12154 |
| chr2 | 75,819,164 | 75,862,697 | 75,819,164 | 75,862,697 | JCH | Null genotypes | NA18555 |
| chr2 | 77,951,799 | 77,968,752 | 77,951,799 | 77,968,752 | JCH | Null genotypes | NA18998 |
| chr2 | 87,388,752 | 87,418,656 | 87,391,624 | 87,418,656 | CEU | Mendelian inconsistencies | (NA12146, NA10847), (NA12239, NA10847) |
| | | | 87,388,752 | 87,399,350 | CEU | Mendelian inconsistencies | (NA12239, NA10847) |
| chr2 | 87,448,659 | 87,465,271 | 87,448,659 | 87,465,271 | CEU | Mendelian inconsistencies | (NA12239, NA10847), (NA07055, NA07048) |
| chr2 | 89,039,268 | 89,049,267 | 89,039,268 | 89,049,267 | CEU | Null genotypes | NA12005, NA10839, NA10855, NA11992, |
| | | | | | | | NA11993, NA11994, NA12236, NA06994, |
| | | | | | | | NA07000, NA11839, NA12044, NA12707, |
| | | | | | | | NA12872, NA06993, NA06985, NA12248, |
| | | | | | | | NA10835 |
| chr2 | 89,093,935 | 89,175,498 | 89,093,935 | 89,175,498 | CEU | Null genotypes | NA12005, NA10839, NA10856, NA10855, |
| | | | | | | | NA12236, NA10830, NA06994, NA11839, |
| | | | | | | | NA12872, NA12873, NA12760, NA07048, |
| | | | | | | | NA06993, NA12248, NA10835 |
| chr2 | 89,796,705 | 90,026,105 | 89,866,750 | 89,993,748 | CEU | Null genotypes | NA10831 |
| | | | 89,826,086 | 89,981,417 | YRI | Null genotypes | NA18500, NA18505 |
| | | | 89,796,705 | 90,026,105 | YRI | Null genotypes | NA18500, NA18505, NA19137 |
| | | | 89,803,197 | 89,848,524 | YRI | Null genotypes | NA18505 |
| | | | 89,823,734 | 89,997,623 | CEU | Mendelian inconsistencies | (NA12814, NA12802), (NA12875, NA12865), |
| | | | | | | | (NA06993, NA06991) |
| | | | 89,878,485 | 89,992,924 | CEU | Mendelian inconsistencies | (NA06993, NA06991) |
| chr2 | 98,493,439 | 98,507,325 | 98,493,439 | 98,507,325 | YRI | Mendelian inconsistencies | (NA18523, NA18521) |
| chr2 | 108,165,482 | 108,177,918 | 108,165,482 | 108,177,918 | YRI | Null genotypes | NA18872 |
| chr2 | 108,209,110 | 108,228,500 | 108,209,110 | 108,228,500 | YRI | Null genotypes | NA18872 |
| chr2 | 112,154,976 | 112,156,194 | 112,154,976 | 112,156,194 | CEU | Mendelian inconsistencies | (NA07345, NA07348) |
| chr2 | 123,575,969 | 123,577,446 | 123,575,969 | 123,577,446 | JCH | Null genotypes | NA18545, NA18558, NA18537, NA18540, |
| | | | | | | | NA18633, NA18635, NA18577, NA18594, |
| | | | | | | | NA19000, NA18971 |
| chr2 | 129,734,701 | 129,735,219 | 129,734,701 | 129,735,219 | JCH | Null genotypes | NA18564, NA18566, NA18579, NA18570, |
| | | | | | | | NA18968, NA18952 |
| chr2 | 132,397,741 | 132,437,832 | 132,397,741 | 132,437,832 | YRI | Null genotypes | NA19140 |
| chr2 | 147,075,728 | 147,086,685 | 147,075,728 | 147,086,685 | CEU | Null genotypes | NA11829, NA11832, NA12154, NA12155, |
| | | | | | | | NA07348, NA12144, NA10846, NA12234, |
| | | | | | | | NA12878, NA12875, NA12865, NA12760, |
| | | | | | | | NA07034, NA07055, NA07048, NA11882, |
| | | | | | | | NA10859 |
| | | | 147,075,728 | 147,085,187 | JCH | Null genotypes | NA18547, NA18609, NA18550, NA18564, |
| | | | | | | | NA18542, NA18532, NA18603, NA18540, |
| | | | | | | | NA18566, NA18579, NA18582, NA18633, |
| | | | | | | | NA18570, NA18612, NA18571, NA18620, |
| | | | | | | | NA18621, NA18573, NA18953, NA18969, |
| | | | | | | | NA18961, NA18972, NA18964, NA18956, |
| | | | | | | | NA18943, NA18948, NA18966, NA18975, |
| | | | | | | | NA18994, NA18992, NA18990, NA18987, |
| | | | | | | | NA18980, NA18995, NA18971, NA18974, |
| | | | | | | | NA19003 |
| | | | 147,075,728 | 147,085,187 | JCH | Hardy-Weinberg | population |
| chr2 | 150,928,553 | 150,929,911 | 150,928,553 | 150,929,911 | CEU | Mendelian inconsistencies | (NA07055, NA07048) |
| chr2 | 152,066,616 | 152,072,353 | 152,066,616 | 152,072,353 | YRI | Mendelian inconsistencies | (NA19203, NA19205) |
| chr2 | 152,524,640 | 152,533,963 | 152,524,640 | 152,533,963 | YRI | Mendelian inconsistencies | (NA19203, NA19205) |
| chr2 | 185,330,664 | 185,336,780 | 185,330,664 | 185,336,780 | YRI | Mendelian inconsistencies | (NA19137, NA19139) |
| chr2 | 185,454,384 | 185,455,058 | 185,454,384 | 185,455,058 | YRI | Mendelian inconsistencies | (NA18501, NA18500), (NA18505, NA18503) |
| chr2 | 196,183,303 | 196,184,993 | 196,183,303 | 196,184,993 | YRI | Null genotypes | NA18858, NA18854, NA18853, NA18863, |
| | | | | | | | NA18914, NA19202, NA19207, NA19140, |
| | | | | | | | NA19098, NA19192 |
| | | | 196,183,303 | 196,184,993 | JCH | Null genotypes | NA18542, NA18570 |
| chr2 | 203,499,611 | 203,511,609 | 203,499,611 | 203,511,609 | YRI | Mendelian inconsistencies | (NA18505, NA18503), (NA18507, NA18506), |
| | | | | | | | (NA19201, NA19202), (NA19172, NA19173), |
| | | | | | | | (NA19203, NA19205), (NA19206, NA19208), |
| | | | | | | | (NA19223, NA19221) |
| chr2 | 208,555,606 | 208,560,381 | 208,555,606 | 208,560,381 | YRI | Mendelian inconsistencies | (NA18852, NA18854) |
| chr2 | 209,437,916 | 209,442,285 | 209,437,916 | 209,442,285 | YRI | Mendelian inconsistencies | (NA18502, NA18500) |
| chr2 | 233,430,372 | 233,455,176 | 233,430,372 | 233,455,176 | JCH | Null genotypes | NA18622 |
| chr2 | 238,086,689 | 238,093,885 | 238,086,689 | 238,093,885 | JCH | Null genotypes | NA18592, NA18637 |
| chr2 | 241,763,283 | 241,775,378 | 241,763,283 | 241,775,378 | JCH | Null genotypes | NA18942 |
| chr2 | 243,342,997 | 243,363,765 | 243,342,997 | 243,363,765 | JCH | Null genotypes | NA18605 |
| chr3 | 4,063,576 | 4,076,356 | 4,063,576 | 4,076,356 | CEU | Mendelian inconsistencies | (NA12873, NA12864) |
| chr3 | 5,073,705 | 5,078,825 | 5,073,705 | 5,078,825 | JCH | Null genotypes | NA18961, NA18981 |
| chr3 | 6,196,030 | 6,211,038 | 6,196,030 | 6,211,038 | YRI | Mendelian inconsistencies | (NA18859, NA18860) |
| chr3 | 15,366,898 | 15,373,051 | 15,366,898 | 15,373,051 | JCH | Null genotypes | NA18582, NA18966, NA18990 |
| chr3 | 16,246,804 | 16,248,562 | 16,246,804 | 16,248,562 | CEU | Mendelian inconsistencies | (NA12144, NA10846) |
| chr3 | 22,058,201 | 22,060,774 | 22,058,201 | 22,060,774 | YRI | Mendelian inconsistencies | (NA18505, NA18503) |
| chr3 | 22,323,109 | 22,338,851 | 22,323,109 | 22,338,851 | YRI | Mendelian inconsistencies | (NA18912, NA18914) |
| chr3 | 30,167,383 | 30,170,752 | 30,167,383 | 30,170,752 | JCH | Null genotypes | NA18529, NA18579, NA18570, NA18945, |
| | | | | | | | NA18978 |
| chr3 | 35,935,059 | 35,957,498 | 35,935,059 | 35,957,498 | YRI | Null genotypes | NA19194 |
| chr3 | 35,977,058 | 36,016,014 | 35,977,058 | 36,016,014 | JCH | Null genotypes | NA18547, NA18943, NA18947, NA18944 |
| chr3 | 46,758,432 | 46,807,284 | 46,758,432 | 46,807,284 | YRI | Mendelian inconsistencies | (NA18501, NA18500), (NA18505, NA18503), |
| | | | | | | | (NA18859, NA18860), (NA19152, NA19154) |
| | | | 46,758,432 | 46,807,284 | YRI | Hardy-Weinberg | population |
| chr3 | 52,991,144 | 52,995,043 | 52,991,144 | 52,995,043 | CEU | Mendelian inconsistencies | (NA12812, NA12801) |
| chr3 | 60,783,767 | 60,860,449 | 60,816,034 | 60,842,631 | CEU | Null genotypes | NA11992, NA10835 |
| | | | 60,783,767 | 60,860,449 | CEU | Mendelian inconsistencies | (NA11992, NA10860) |
| | | | 60,806,084 | 60,844,635 | CEU | Mendelian inconsistencies | (NA11992, NA10860) |
| | | | 60,833,986 | 60,838,247 | CEU | Mendelian inconsistencies | (NA11992, NA10860) |
| chr3 | 62,136,076 | 62,139,994 | 62,136,076 | 62,139,994 | JCH | Null genotypes | NA18576, NA18960, NA18980 |
| chr3 | 65,155,877 | 65,169,617 | 65,155,877 | 65,169,617 | CEU | Mendelian inconsistencies | (NA12043, NA10857), (NA12248, NA10835) |
| | | | 65,156,319 | 65,158,131 | CEU | Mendelian inconsistencies | (NA07055, NA07048), (NA12248, NA10835) |
| chr3 | 68,023,881 | 68,025,905 | 68,023,881 | 68,025,905 | JCH | Null genotypes | NA18940, NA18967 |
| chr3 | 84,621,613 | 84,622,376 | 84,621,613 | 84,622,376 | JCH | Null genotypes | NA18576, NA18961, NA18964, NA18948, |
| | | | | | | | NA18987, NA19003 |
| chr3 | 89,323,763 | 89,337,965 | 89,323,763 | 89,337,965 | JCH | Null genotypes | NA19007 |
| | | | 89,328,157 | 89,337,965 | CEU | Mendelian inconsistencies | (NA12761, NA12752) |
| | | | 89,168,782 | 89,401,166 | JCH | Hardy-Weinberg | population |
| chr3 | 89,594,730 | 89,596,771 | 89,594,730 | 89,596,771 | YRI | Mendelian inconsistencies | (NA19238, NA19240) |
| chr3 | 99,731,950 | 99,732,552 | 99,731,950 | 99,732,552 | CEU | Null genotypes | NA12006, NA10839, NA11831, NA11995, |
| | | | | | | | NA12155, NA11840, NA12239, NA12891, |
| | | | | | | | NA12892, NA12878, NA12814, NA12802, |
| | | | | | | | NA12875, NA12761, NA12763, NA07034, |
| | | | | | | | NA07048 |
| | | | 99,731,950 | 99,732,552 | JCH | Null genotypes | NA18609, NA18564, NA18561, NA18579, |
| | | | | | | | NA18635, NA18636, NA18593, NA18621, |
| | | | | | | | NA18942, NA18968, NA18969, NA18951, |
| | | | | | | | NA18945, NA18949, NA18948, NA18952, |
| | | | | | | | NA18999, NA19007, NA18990, NA18987, |
| | | | | | | | NA18976, NA18971 |
| | | | 99,731,950 | 99,732,552 | YRI | Mendelian inconsistencies | (NA19160, NA19161) |
| chr3 | 100,265,427 | 100,268,749 | 100,265,427 | 100,268,749 | CEU | Mendelian inconsistencies | (NA11831, NA10855) |
| | | | 100,264,911 | 100,268,749 | CEU | Hardy-Weinberg | population |
| chr3 | 105,453,223 | 105,468,789 | 105,453,223 | 105,468,789 | JCH | Null genotypes | NA18966 |
| chr3 | 105,563,293 | 105,578,251 | 105,563,293 | 105,578,251 | JCH | Null genotypes | NA18966 |
| chr3 | 105,679,895 | 105,708,632 | 105,679,895 | 105,708,632 | JCH | Null genotypes | NA18966 |
| chr3 | 105,741,486 | 105,749,182 | 105,741,486 | 105,749,182 | JCH | Null genotypes | NA18966 |
| chr3 | 105,820,171 | 105,864,826 | 105,820,171 | 105,864,826 | JCH | Null genotypes | NA18966 |
| chr3 | 105,938,874 | 105,952,504 | 105,938,874 | 105,952,504 | JCH | Null genotypes | NA18966 |
| chr3 | 106,031,726 | 106,098,859 | 106,031,726 | 106,098,859 | JCH | Null genotypes | NA18966 |
| chr3 | 115,979,707 | 115,988,797 | 115,979,707 | 115,988,797 | YRI | Null genotypes | NA18515, NA18517, NA18516, NA18872, |
| | | | | | | | NA18871, NA19205, NA19203, NA19161, |
| | | | | | | | NA19160, NA19132, NA19130, NA19194, |
| | | | | | | | NA19192 |
| | | | 115,979,707 | 115,981,957 | YRI | Mendelian inconsistencies | (NA18507, NA18506) |
| chr3 | 127,035,541 | 127,042,413 | 127,035,541 | 127,042,413 | YRI | Null genotypes | NA19132, NA19131, NA19130 |
| chr3 | 131,109,842 | 131,114,005 | 131,109,842 | 131,114,005 | JCH | Null genotypes | NA18572, NA18545, NA18635, NA18621, |
| | | | | | | | NA18594, NA18622, NA18949 |
| chr3 | 131,453,760 | 131,454,529 | 131,453,760 | 131,454,529 | CEU | Mendelian inconsistencies | (NA12234, NA10863) |
| chr3 | 133,032,620 | 133,033,926 | 133,032,620 | 133,033,926 | CEU | Null genotypes | NA12003, NA12154, NA10830, NA07348, |
| | | | | | | | NA07000, NA12891, NA12872, NA12874, |
| | | | | | | | NA12865, NA12763, NA07034, NA07048 |
| chr3 | 133,312,348 | 133,312,893 | 133,312,348 | 133,312,893 | JCH | Null genotypes | NA18550, NA18558, NA18633 |
| chr3 | 138,351,092 | 138,352,662 | 138,351,092 | 138,352,662 | YRI | Mendelian inconsistencies | (NA19102, NA19103) |
| chr3 | 143,931,519 | 143,932,566 | 143,931,519 | 143,932,566 | YRI | Mendelian inconsistencies | (NA18913, NA18914), (NA19238, NA19240) |
| chr3 | 150,285,601 | 150,288,244 | 150,285,601 | 150,288,244 | CEU | Mendelian inconsistencies | (NA12234, NA10863) |
| chr3 | 153,671,436 | 153,675,234 | 153,671,436 | 153,675,234 | CEU | Mendelian inconsistencies | (NA11992, NA10860) |
| chr3 | 156,501,087 | 156,505,963 | 156,501,087 | 156,505,963 | YRI | Mendelian inconsistencies | (NA18516, NA18515), (NA18870, NA18872), |
| | | | | | | | (NA19137, NA19139), (NA19200, NA19202), |
| | | | | | | | (NA19206, NA19208), (NA19160, NA19161) |
| chr3 | 160,095,253 | 160,113,199 | 160,095,253 | 160,113,199 | CEU | Null genotypes | NA07348 |
| chr3 | 163,450,906 | 163,462,798 | 163,450,906 | 163,462,798 | CEU | Mendelian inconsistencies | (NA11840, NA10854) |
| chr3 | 163,538,907 | 163,550,497 | 163,538,907 | 163,550,497 | JCH | Null genotypes | NA18540, NA18968, NA18969, NA18966, |
| | | | | | | | NA18991 |
| chr3 | 163,833,596 | 163,943,569 | 163,860,609 | 163,861,908 | CEU | Null genotypes | NA12003, NA12005, NA12006, NA10839, |
| | | | | | | | NA12056, NA12057, NA10851, NA11830, |
| | | | | | | | NA11831, NA10860, NA11994, NA10861, |
| | | | | | | | NA10831, NA07357, NA07345, NA07348, |
| | | | | | | | NA06994, NA07029, NA07056, NA11839, |
| | | | | | | | NA12044, NA12145, NA12264, NA12716, |
| | | | | | | | NA12717, NA12707, NA12891, NA12892, |
| | | | | | | | NA12878, NA12812, NA12801, NA12872, |
| | | | | | | | NA12874, NA12875, NA12865, NA12752, |
| | | | | | | | NA12763, NA12750, NA12751, NA12740, |
| | | | | | | | NA11882, NA12248, NA12249, NA10835 |
| | | | 163,882,205 | 163,926,256 | CEU | Null genotypes | NA12003, NA12005, NA12057, NA10851, |
| | | | | | | | NA07357, NA07056, NA12264, NA12891, |
| | | | | | | | NA12801, NA12874, NA12752, NA12750, |
| | | | | | | | NA12740 |
| | | | 163,833,596 | 163,943,569 | YRI | Null genotypes | NA18505, NA18860, NA18857, NA18855, |
| | | | | | | | NA18863, NA18861, NA19103, NA19101, |
| | | | | | | | NA19139, NA19138, NA19200, NA19204, |
| | | | | | | | NA19211, NA19209, NA19208, NA19207, |
| | | | | | | | NA19161, NA19221, NA19222, NA19223, |
| | | | | | | | NA19116, NA19154, NA19152, NA19100, |
| | | | | | | | NA19098 |
| | | | 163,857,897 | 163,861,908 | JCH | Null genotypes | NA18524, NA18572, NA18547, NA18609, |
| | | | | | | | NA18550, NA18552, NA18611, NA18555, |
| | | | | | | | NA18542, NA18532, NA18561, NA18603, |
| | | | | | | | NA18540, NA18605, NA18566, NA18563, |
| | | | | | | | NA18624, NA18579, NA18632, NA18582, |
| | | | | | | | NA18633, NA18635, NA18592, NA18636, |
| | | | | | | | NA18593, NA18576, NA18570, NA18612, |
| | | | | | | | NA18571, NA18620, NA18621, NA18594, |
| | | | | | | | NA18622, NA18573, NA18623, NA18637, |
| | | | | | | | NA18526, NA18942, NA18953, NA18968, |
| | | | | | | | NA18959, NA18960, NA18972, NA18965, |
| | | | | | | | NA18956, NA18940, NA18951, NA18943, |
| | | | | | | | NA18947, NA18944, NA18945, NA18949, |
| | | | | | | | NA18948, NA18966, NA18975, NA18991, |
| | | | | | | | NA18992, NA18997, NA18998, NA19005, |
| | | | | | | | NA18999, NA19007, NA18990, NA18987, |
| | | | | | | | NA18967, NA18976, NA18978, |
| | | | | | | | NA18995, NA18981, NA18971, NA18974, |
| | | | | | | | NA19003 |
| | | | 163,837,185 | 163,940,699 | JCH | Null genotypes | NA18524, NA18572, NA18547, NA18609, |
| | | | | | | | NA18550, NA18552, NA18555, NA18542, |
| | | | | | | | NA18532, NA18561, NA18603, NA18540, |
| | | | | | | | NA18605, NA18566, NA18563, NA18624, |
| | | | | | | | NA18579, NA18632, NA18582, NA18633, |
| | | | | | | | NA18635, NA18592, NA18636, NA18593, |
| | | | | | | | NA18576, NA18570, NA18612, NA18571, |
| | | | | | | | NA18620, NA18621, NA18594, NA18622, |
| | | | | | | | NA18573, NA18623, NA18637, NA18953, |
| | | | | | | | NA18968, NA18959, NA18960, NA18972, |
| | | | | | | | NA18965, NA18956, NA18940, NA18951, |
| | | | | | | | NA18943, NA18944, NA18945, NA18949, |
| | | | | | | | NA18948, NA18991, NA18992, NA18997, |
| | | | | | | | NA18998, NA19005, NA19007, NA18990, |
| | | | | | | | NA18987, NA18967, NA18976, NA18978, |
| | | | | | | | NA18995, NA18981, NA18971, NA18974, |
| | | | | | | | NA19003 |
| | | | 163,835,144 | 163,922,881 | CEU | Mendelian inconsistencies | (NA07000, NA07029) |
| | | | 163,835,144 | 163,891,949 | YRI | Mendelian inconsistencies | (NA19140, NA19142), (NA19238, NA19240) |
| | | | 163,837,185 | 163,917,427 | CEU | Mendelian inconsistencies | (NA12003, NA10838), (NA07000, NA07029), |
| | | | | | | | (NA07056, NA07019), (NA12264, NA10863), |
| | | | | | | | (NA12813, NA12801), (NA12760, NA12752), |
| | | | | | | | (NA12761, NA12752) |
| | | | 163,875,766 | 163,940,699 | CEU | Mendelian inconsistencies | (NA12005, NA10839), (NA12056, NA10851), |
| | | | | | | | (NA07357, NA07348), (NA07056, NA07019), |
| | | | | | | | (NA12264, NA10863), (NA12891, NA12878), |
| | | | | | | | (NA12874, NA12865), (NA12760, NA12752), |
| | | | | | | | (NA12761, NA12752), (NA12751, NA12740) |
| | | | 163,840,486 | 163,939,798 | CEU | Mendelian inconsistencies | (NA12005, NA10839), (NA12056, NA10851), |
| | | | | | | | (NA07357, NA07348), (NA07000, NA07029), |
| | | | | | | | (NA12891, NA12878), (NA12812, NA12801), |
| | | | | | | | (NA12874, NA12865), (NA12751, NA12740) |
| | | | 163,860,189 | 163,934,963 | CEU | Mendelian inconsistencies | (NA12146, NA10847), (NA07034, NA07048), |
| | | | | | | | (NA06985, NA06991) |
| | | | 163,833,596 | 163,892,785 | CEU | Mendelian inconsistencies | (NA07000, NA07029) |
| | | | 163,837,185 | 163,943,569 | YRI | Mendelian inconsistencies | (NA19140, NA19142), (NA19238, NA19240) |
| | | | 163,888,838 | 163,903,159 | YRI | Mendelian inconsistencies | (NA19144, NA19145), (NA19238, NA19240) |
| | | | 163,889,909 | 163,912,703 | YRI | Mendelian inconsistencies | (NA19140, NA19142), (NA19144, NA19145) |
| | | | 163,833,596 | 163,943,569 | CEU | Hardy-Weinberg | population |
| | | | 163,837,185 | 163,943,569 | YRI | Hardy-Weinberg | population |
| | | | 163,826,348 | 163,940,699 | JCH | Hardy-Weinberg | population |
| | | | 163,835,144 | 163,922,881 | CEU | Hardy-Weinberg | population |
| | | | 163,835,144 | 163,922,881 | JCH | Hardy-Weinberg | population |
| chr3 | 164,983,304 | 164,985,198 | 164,983,304 | 164,985,198 | CEU | Mendelian inconsistencies | (NA07056, NA07019) |
| chr3 | 166,377,266 | 166,386,073 | 166,377,266 | 166,386,073 | CEU | Mendelian inconsistencies | (NA11881, NA10859) |
| chr3 | 166,635,288 | 166,641,054 | 166,635,288 | 166,641,054 | CEU | Null genotypes | NA07056, NA11840, NA10863, NA12801, |
| | | | | | | | NA12740 |
| chr3 | 176,402,880 | 176,404,312 | 176,402,880 | 176,404,312 | JCH | Null genotypes | NA18622, NA18974 |
| chr3 | 177,217,491 | 177,250,587 | 177,217,491 | 177,250,587 | YRI | Null genotypes | NA18523 |
| chr3 | 180,609,192 | 180,611,006 | 180,609,192 | 180,611,006 | CEU | Mendelian inconsistencies | (NA12057, NA10851) |
| chr3 | 187,710,472 | 187,716,485 | 187,710,472 | 187,716,485 | CEU | Mendelian inconsistencies | (NA12813, NA12801) |
| | | | 187,710,472 | 187,711,540 | CEU | Mendelian inconsistencies | (NA12813, NA12801) |
| chr3 | 190,685,007 | 190,688,562 | 190,685,007 | 190,688,562 | YRI | Null genotypes | NA18503, NA18504, NA19211 |
| | | | 190,685,007 | 190,688,562 | JCH | Null genotypes | NA18547, NA18609, NA18570, NA18612, |
| | | | | | | | NA18944, NA18949 |
| chr3 | 191,058,267 | 191,060,542 | 191,058,267 | 191,060,542 | YRI | Null genotypes | NA18858, NA18857, NA18914, N |
| | | | | | | | NA19205, NA19127, NA19132, NA19240 |
| | | | 191,058,267 | 191,060,542 | JCH | Null genotypes | NA18524, NA18611, NA18537, NA18563, |
| | | | | | | | NA18624, NA18579, NA18633, NA18592, |
| | | | | | | | NA18620, NA18623, NA18953, NA18969, |
| | | | | | | | NA18991, NA19000, NA18976 |
| chr3 | 192,388,157 | 192,390,897 | 192,388,157 | 192,390,897 | YRI | Null genotypes | NA19101 |
| | | | 192,388,776 | 192,390,897 | YRI | Mendelian inconsistencies | (NA19159, NA19161), (NA19193, NA19194) |
| | | | 192,388,157 | 192,390,897 | CEU | Hardy-Weinberg | population |
| chr3 | 193,920,249 | 193,930,629 | 193,920,249 | 193,930,629 | YRI | Null genotypes | NA19210 |
| chr3 | 194,196,286 | 194,205,086 | 194,196,286 | 194,201,998 | YRI | Null genotypes | NA18860, NA18858, NA18859, NA18521, |
| | | | | | | | NA18522, NA18854, NA18852, NA18853, |
| | | | | | | | NA18857, NA19102, NA19139, NA19137, |
| | | | | | | | NA19138, NA19204, NA19211, NA19120, |
| | | | | | | | NA19142, NA19140, NA19145, NA19143, |
| | | | | | | | NA19128, NA19132, NA19131, NA19100, |
| | | | | | | | NA19238 |
| | | | 194,196,286 | 194,201,998 | JCH | Null genotypes | NA18609, NA18550, NA18608, NA18552, |
| | | | | | | | NA18611, NA18564, NA18545, NA18542, |
| | | | | | | | NA18558, NA18562, NA18537, NA18603, |
| | | | | | | | NA18540, NA18563, NA18624, NA18579, |
| | | | | | | | NA18632, NA18582, NA18633, NA18635, |
| | | | | | | | NA18592, NA18636, NA18593, NA18577, |
| | | | | | | | NA18570, NA18612, NA18571, NA18620, |
| | | | | | | | NA18594, NA18622, NA18623, NA18637, |
| | | | | | | | NA18526, NA18942, NA18953, NA18959, |
| | | | | | | | NA18969, NA18961, NA18972, NA18965, |
| | | | | | | | NA18973, NA18964, NA18940, NA18951, |
| | | | | | | | NA18943, NA18947, NA18944, NA18945, |
| | | | | | | | NA18948, NA18952, NA18966, NA18975, |
| | | | | | | | NA18991, NA18994, NA18992, NA18997, |
| | | | | | | | NA18998, NA19000, NA19005, NA19007, |
| | | | | | | | NA18990, NA18987, NA18967, NA18976, |
| | | | | | | | NA18978, NA18970, NA18980, NA18981, |
| | | | | | | | NA18971, NA18974 |
| | | | 194,200,830 | 194,204,450 | CEU | Null genotypes | NA12005, NA12056, NA10851, NA11830, |
| | | | | | | | NA11831, NA11832, NA10855, NA11993, |
| | | | | | | | NA12155, NA07357, NA06994, NA07000, |
| | | | | | | | NA07029, NA07022, NA07019, NA12043, |
| | | | | | | | NA10857, NA12144, NA12146, NA12239, |
| | | | | | | | NA10847, NA12264, NA12234, NA10863, |
| | | | | | | | NA12716, NA12717, NA12707, NA12891, |
| | | | | | | | NA12812, NA12801, NA12872, NA12864, |
| | | | | | | | NA12875, NA12752, NA12763, NA07034, |
| | | | | | | | NA07048, NA12750, NA12248, NA10835 |
| | | | 194,196,286 | 194,205,086 | CEU | Mendelian inconsistencies | (NA12004, NA10838), (NA12144, NA10846), |
| | | | | | | | (NA12813, NA12801), (NA12875, NA12865), |
| | | | | | | | (NA12760, NA12752) |
| | | | 194,196,286 | 194,201,998 | YRI | Hardy-Weinberg | population |
| chr3 | 194,457,389 | 194,459,618 | 194,457,389 | 194,459,618 | JCH | Null genotypes | NA18637, NA18944, NA18945, NA18992, |
| | | | | | | | NA19000, NA18987 |
| chr4 | 9,877,861 | 9,879,029 | 9,877,861 | 9,879,029 | CEU | Mendelian inconsistencies | (NA12154, NA10830) |
| chr4 | 9,969,524 | 9,980,122 | 9,969,524 | 9,980,122 | CEU | Null genotypes | NA12003, NA12004, NA10838, NA12005, |
| | | | | | | | NA10839, NA12056, NA12057, NA10851, |
| | | | | | | | NA11829, NA11830, NA10856, NA11992, |
| | | | | | | | NA11993, NA10860, NA11994, NA11995, |
| | | | | | | | NA10861, NA12156, NA10831, NA07345, |
| | | | | | | | NA07022, NA07056, NA07019, NA11839, |
| | | | | | | | NA12146, NA12239, NA10847, NA12234, |
| | | | | | | | NA12716, NA12717, NA12707, NA12892, |
| | | | | | | | NA12812, NA12815, NA12873, NA12761, |
| | | | | | | | NA12762, NA12763, NA12753, NA07055, |
| | | | | | | | NA06993, NA06985, NA06991, NA12750, |
| | | | | | | | NA12248, NA12249, NA10835 |
| | | | 9,969,524 | 9,980,122 | YRI | Null genotypes | NA18504, NA18515, NA18523, NA18871, |
| | | | | | | | NA18852, NA18855, NA18861, NA18914, |
| | | | | | | | NA18912, NA19092, NA19103, NA19101, |
| | | | | | | | NA19205, NA19120, NA19116, NA19119, |
| | | | | | | | NA19140, NA19127, NA19098 |
| | | | 9,969,524 | 9,980,122 | JCH | Null genotypes | NA18524, NA18545, NA18561, NA18632, |
| | | | | | | | NA18636, NA18571, NA18594, NA18637, |
| | | | | | | | NA18959, NA18969, NA18961, NA18964, |
| | | | | | | | NA18956, NA18947, NA18945, NA18948, |
| | | | | | | | NA18966, NA18975, NA18992, NA18998, |
| | | | | | | | NA19005, NA18999, NA18990, NA18987, |
| | | | | | | | NA18976, NA18995 |
| | | | 9,969,524 | 9,980,122 | YRI | Mendelian inconsistencies | (NA19152, NA19154), (NA19143, NA19145) |
| | | | 9,969,524 | 9,980,122 | YRI | Hardy-Weinberg | population |
| | | | 9,969,524 | 9,998,131 | JCH | Hardy-Weinberg | population |
| chr4 | 10,148,210 | 10,151,039 | 10,148,210 | 10,151,039 | JCH | Null genotypes | NA18971 |
| | | | 10,148,210 | 10,151,039 | CEU | Null genotypes | NA11831, NA11832, NA10855, NA12236, |
| | | | | | | | NA11840, NA12044, NA10857, NA10846 |
| chr4 | 12,125,940 | 12,134,830 | 12,125,940 | 12,130,201 | YRI | Mendelian inconsistencies | (NA18861, NA18863) |
| | | | 12,129,939 | 12,134,830 | YRI | Mendelian inconsistencies | (NA18861, NA18863) |
| chr4 | 20,312,963 | 20,313,991 | 20,312,963 | 20,313,991 | YRI | Null genotypes | NA19161 |
| chr4 | 21,123,929 | 21,126,700 | 21,123,929 | 21,126,700 | YRI | Null genotypes | NA18506, NA18508, NA18854, NA18855, |
| | | | | | | | NA18913, NA19094, NA19092, NA19103, |
| | | | | | | | NA19201, NA19205, NA19210, NA19159, |
| | | | | | | | NA19222, NA19119, NA19145, NA19143, |
| | | | | | | | NA19144, NA19192, NA19238 |
| | | | 21,043,096 | 21,126,700 | YRI | Hardy-Weinberg | population |
| chr4 | 32,163,126 | 32,169,581 | 32,163,126 | 32,169,581 | JCH | Null genotypes | NA18945, NA18949 |
| chr4 | 34,677,422 | 34,724,191 | 34,677,422 | 34,722,072 | CEU | Null genotypes | NA11832, NA11992, NA10860, NA12156, |
| | | | | | | | NA12878, NA07034, NA10859 |
| | | | 34,677,422 | 34,724,191 | YRI | Null genotypes | NA19200, NA19098 |
| | | | 34,677,422 | 34,724,191 | JCH | Null genotypes | NA18529 |
| | | | 34,685,154 | 34,722,072 | CEU | Mendelian inconsistencies | (NA12815, NA12802) |
| | | | 34,685,154 | 34,701,647 | YRI | Mendelian inconsistencies | (NA18858, NA18860), (NA19131, NA19132) |
| | | | 34,716,060 | 34,724,191 | YRI | Mendelian inconsistencies | (NA18858, NA18860), (NA19206, NA19208) |
| | | | 34,686,467 | 34,707,485 | YRI | Mendelian inconsistencies | (NA19131, NA19132) |
| | | | 34,685,154 | 34,762,467 | CEU | Hardy-Weinberg | population |
| chr4 | 54,366,717 | 54,399,111 | 54,366,717 | 54,399,111 | CEU | Null genotypes | NA12802 |
| chr4 | 63,672,955 | 63,678,558 | 63,672,955 | 63,678,558 | JCH | Null genotypes | NA18609, NA18545, NA18542, NA18532, |
| | | | | | | | NA18537, NA18540, NA18635, NA18960, |
| | | | | | | | NA18961, NA18972, NA18965, NA18945, |
| | | | | | | | NA18949, NA18952, NA18966, NA18975, |
| | | | | | | | NA18998 |
| | | | 63,672,955 | 63,678,558 | CEU | Null genotypes | NA07000, NA07029, NA12761 |
| | | | 63,672,955 | 63,676,155 | YRI | Mendelian inconsistencies | (NA18502, NA18500) |
| chr4 | 64,701,164 | 64,713,008 | 64,701,164 | 64,712,923 | JCH | Null genotypes | NA18542, NA18995 |
| | | | 64,701,164 | 64,712,923 | CEU | Null genotypes | NA12154, NA07056, NA07019, NA12234, |
| | | | | | | | NA12761, NA12762, NA06993, NA06991, |
| | | | | | | | NA12249 |
| | | | 64,702,559 | 64,713,008 | CEU | Null genotypes | NA12154, NA07056, NA07019, NA12234, |
| | | | | | | | NA12761, NA12762, NA06993, NA06991, |
| | | | | | | | NA12249 |
| | | | 64,707,387 | 64,713,008 | JCH | Null genotypes | NA18542, NA18995 |
| | | | 64,701,164 | 64,712,923 | CEU | Mendelian inconsistencies | (NA12239, NA10847), (NA12872, NA12864), |
| | | | | | | | (NA12750, NA12740) |
| | | | 64,701,164 | 64,712,923 | CEU | Hardy-Weinberg | population |
| chr4 | 69,377,786 | 69,808,237 | 69,432,417 | 69,486,334 | YRI | Null genotypes | NA19172, NA19161, NA19160, NA19098 |
| | | | 69,377,786 | 69,808,237 | JCH | Null genotypes | NA18572, NA18547, NA18608, NA18552, |
| | | | | | | | NA18611, NA18545, NA18558, NA18532, |
| | | | | | | | NA18561, NA18562, NA18537, NA18603, |
| | | | | | | | NA18540, NA18605, NA18566, NA18563, |
| | | | | | | | NA18624, NA18579, NA18632, NA18582, |
| | | | | | | | NA18633, NA18635, NA18592, NA18636, |
| | | | | | | | NA18593, NA18576, NA18570, NA18571, |
| | | | | | | | NA18620, NA18594, NA18622, NA18623, |
| | | | | | | | NA18526, NA18942, NA18953, NA18968, |
| | | | | | | | NA18959, NA18969, NA18961, NA18972, |
| | | | | | | | NA18973, NA18964, NA18940, NA18947, |
| | | | | | | | NA18952, NA18966, NA18975, NA18991, |
| | | | | | | | NA18994, NA18992, NA18997, NA18998, |
| | | | | | | | NA19005, NA18999, NA19007, NA18990, |
| | | | | | | | NA18987, NA18967, NA18976, NA18970, |
| | | | | | | | NA18980, NA18995, NA18981, NA18971 |
| | | | 69,378,123 | 69,808,237 | CEU | Null genotypes | NA12056, NA12057, NA10851, NA10831, |
| | | | | | | | NA12264, NA12716, NA12892, NA12878, |
| | | | | | | | NA12813 |
| | | | 69,460,790 | 69,486,227 | YRI | Null genotypes | NA19172, NA19161, NA19160, NA19098 |
| | | | 69,460,790 | 69,486,227 | YRI | Mendelian inconsistencies | (NA19143, NA19145) |
| | | | 69,441,695 | 69,482,361 | CEU | Mendelian inconsistencies | (NA11995, NA10861), (NA12043, NA10857), |
| | | | | | | | (NA12145, NA10846), (NA12873, NA12864), |
| | | | | | | | (NA12750, NA12740) |
| | | | 69,393,712 | 69,431,602 | CEU | Mendelian inconsistencies | (NA12145, NA10846), (NA12716, NA12707), |
| | | | | | | | (NA12813, NA12801) |
| | | | 69,450,972 | 69,458,490 | YRI | Mendelian inconsistencies | (NA18858, NA18860), (NA18855, NA18857), |
| | | | | | | | (NA18862, NA18863), (NA19200, NA19202), |
| | | | | | | | (NA19172, NA19173) |
| | | | 69,459,701 | 69,462,910 | YRI | Mendelian inconsistencies | (NA18858, NA18860), (NA19200, NA19202), |
| | | | | | | | (NA19143, NA19145) |
| | | | 69,475,972 | 69,482,770 | YRI | Mendelian inconsistencies | (NA18502, NA18500) |
| | | | 69,482,361 | 69,491,890 | YRI | Mendelian inconsistencies | (NA19143, NA19145) |
| | | | 69,378,123 | 69,482,361 | CEU | Hardy-Weinberg | population |
| | | | 69,431,602 | 69,486,334 | YRI | Hardy-Weinberg | population |
| | | | 69,377,786 | 69,486,334 | JCH | Hardy-Weinberg | population |
| chr4 | 70,447,409 | 70,542,965 | 70,471,691 | 70,542,965 | YRI | Null genotypes | NA18503, NA18504, NA18508, NA18516, |
| | | | | | | | NA18912, NA19094, NA19093, NA19201, |
| | | | | | | | NA19160, NA19119, NA19153, NA19129 |
| | | | 70,471,691 | 70,542,965 | JCH | Null genotypes | NA18965, NA18947 |
| | | | 70,447,409 | 70,542,965 | CEU | Mendelian inconsistencies | (NA12875, NA12865) |
| | | | 70,477,074 | 70,542,965 | YRI | Mendelian inconsistencies | (NA19207, NA19208), (NA19222, NA19221), |
| | | | | | | | (NA19143, NA19145) |
| | | | 70,455,621 | 70,471,691 | YRI | Mendelian inconsistencies | (NA19101, NA19103) |
| | | | 70,412,475 | 70,542,965 | YRI | Hardy-Weinberg | population |
| chr4 | 74,165,317 | 74,224,901 | 74,165,317 | 74,224,901 | CEU | Mendelian inconsistencies | (NA12236, NA10830) |
| | | | 74,174,224 | 74,210,693 | CEU | Mendelian inconsistencies | (NA12236, NA10830) |
| | | | 74,194,506 | 74,195,313 | CEU | Mendelian inconsistencies | (NA12236, NA10830) |
| chr4 | 91,630,184 | 91,656,186 | 91,630,184 | 91,656,186 | CEU | Null genotypes | NA11832 |
| chr4 | 92,391,958 | 92,393,093 | 92,391,958 | 92,393,093 | CEU | Null genotypes | NA12004, NA12057, NA10856, NA11832, |
| | | | | | | | NA11992, NA12154, NA12156, NA07345, |
| | | | | | | | NA07348, NA07056, NA12146, NA12264, |
| | | | | | | | NA10863, NA12802, NA12762 |
| | | | 92,391,958 | 92,393,093 | CEU | Mendelian inconsistencies | (NA12006, NA10839), (NA07000, NA07029), |
| | | | | | | | (NA12812, NA12801), (NA12875, NA12865) |
| chr4 | 94,994,798 | 94,995,548 | 94,994,798 | 94,995,548 | CEU | Mendelian inconsistencies | (NA12145, NA10846) |
| chr4 | 104,670,951 | 104,671,800 | 104,670,951 | 104,671,800 | YRI | Mendelian inconsistencies | (NA18522, NA18521), (NA18870, NA18872) |
| chr4 | 105,212,443 | 105,219,862 | 105,212,443 | 105,219,862 | CEU | Mendelian inconsistencies | (NA12005, NA10839) |
| chr4 | 108,651,560 | 108,665,451 | 108,651,560 | 108,665,451 | YRI | Mendelian inconsistencies | (NA18505, NA18503), (NA18507, NA18506), |
| | | | | | | | (NA18859, NA18860), (NA19201, NA19202), |
| | | | | | | | (NA19130, NA19132) |
| chr4 | 115,637,062 | 115,641,110 | 115,637,062 | 115,641,110 | JCH | Null genotypes | NA18532, NA18635, NA18593, NA18972, |
| | | | | | | | NA18964, NA18945, NA18975, NA18991, |
| | | | | | | | NA18987, NA18976 |
| | | | 115,637,062 | 115,641,110 | CEU | Null genotypes | NA10839, NA11831, NA10855, NA07357, |
| | | | | | | | NA06994, NA07000, NA07029, NA12146, |
| | | | | | | | NA12717, NA12802, NA12760, NA07048, |
| | | | | | | | NA12249 |
| | | | 115,638,909 | 115,641,110 | CEU | Mendelian inconsistencies | (NA12003, NA10838), (NA12056, NA10851), |
| | | | | | | | (NA11830, NA10856), (NA12154, NA10830), |
| | | | | | | | (NA12234, NA10863), (NA12874, NA12865) |
| | | | 115,637,062 | 115,641,110 | YRI | Hardy-Weinberg | population |
| chr4 | 116,620,794 | 116,631,153 | 116,620,794 | 116,631,153 | CEU | Mendelian inconsistencies | (NA12892, NA12878) |
| | | | 116,620,794 | 116,631,153 | YRI | Mendelian inconsistencies | (NA19206, NA19208) |
| chr4 | 119,071,973 | 119,076,909 | 119,071,973 | 119,076,909 | CEU | Mendelian inconsistencies | (NA07000, NA07029) |
| | | | 119,071,973 | 119,076,909 | YRI | Mendelian inconsistencies | (NA19210, NA19211) |
| chr4 | 121,410,025 | 121,411,772 | 121,410,025 | 121,411,772 | CEU | Mendelian inconsistencies | (NA11993, NA10860), (NA12155, NA10831) |
| chr4 | 129,148,757 | 129,162,771 | 129,148,757 | 129,162,771 | YRI | Null genotypes | NA19132, NA19239 |
| chr4 | 130,965,878 | 130,977,227 | 130,965,878 | 130,977,227 | YRI | Mendelian inconsistencies | (NA19239, NA19240) |
| chr4 | 133,046,503 | 133,053,932 | 133,046,503 | 133,053,932 | CEU | Null genotypes | NA06994, NA12872, NA12864 |
| chr4 | 138,551,715 | 138,556,685 | 138,551,715 | 138,556,685 | YRI | Null genotypes | NA18863, NA18861 |
| | | | 138,551,715 | 138,556,685 | CEU | Null genotypes | NA12003, NA10838, NA12154, NA10830, |
| | | | | | | | NA12043, NA12234, NA10863 |
| | | | 138,551,715 | 138,556,270 | CEU | Mendelian inconsistencies | (NA12760, NA12752) |
| | | | 138,551,715 | 138,556,270 | YRI | Mendelian inconsistencies | (NA18871, NA18872), (NA19101, NA19103), |
| | | | | | | | (NA19116, NA19120), (NA19098, NA19100) |
| chr4 | 138,642,484 | 138,644,834 | 138,642,484 | 138,644,834 | CEU | Mendelian inconsistencies | (NA12812, NA12801), (NA11882, NA10859) |
| chr4 | 144,863,310 | 144,873,579 | 144,863,310 | 144,873,579 | YRI | Mendelian inconsistencies | (NA19201, NA19202) |
| chr4 | 152,458,392 | 152,461,345 | 152,458,392 | 152,461,345 | YRI | Null genotypes | NA19211, NA19210, NA19143, NA19239 |
| chr4 | 157,541,319 | 157,545,522 | 157,541,319 | 157,545,522 | CEU | Null genotypes | NA07000 |
| chr4 | 161,637,501 | 161,649,570 | 161,637,501 | 161,649,570 | CEU | Mendelian inconsistencies | (NA12891, NA12878) |
| chr4 | 162,456,693 | 162,462,242 | 162,456,693 | 162,462,242 | JCH | Null genotypes | NA18562, NA18577, NA18594, NA18960, |
| | | | | | | | NA18972, NA18943, NA18975, NA18997, |
| | | | | | | | NA18971 |
| | | | 162,456,693 | 162,462,242 | CEU | Null genotypes | NA07019, NA12802, NA12248 |
| | | | 162,458,627 | 162,462,242 | YRI | Mendelian inconsistencies | (NA19141, NA19142), (NA19131, NA19132) |
| chr4 | 169,519,486 | 169,532,584 | 169,519,486 | 169,532,584 | CEU | Mendelian inconsistencies | (NA12056, NA10851) |
| chr4 | 169,661,623 | 169,683,933 | 169,661,623 | 169,683,933 | CEU | Mendelian inconsistencies | (NA12056, NA10851) |
| chr4 | 170,385,138 | 170,389,969 | 170,385,138 | 170,389,969 | YRI | Null genotypes | NA18852, NA18861 |
| chr4 | 173,685,436 | 173,686,862 | 173,685,436 | 173,686,862 | JCH | Null genotypes | NA18558, NA18632, NA18620, NA19005 |
| | | | 173,685,436 | 173,686,862 | CEU | Null genotypes | NA12236, NA12155, NA10857, NA12145, |
| | | | | | | | NA12239, NA12264, NA12234, NA10863, |
| | | | | | | | NA12892, NA12864, NA12875, NA12760, |
| | | | | | | | NA12762, NA12753, NA07034, NA07055, |
| | | | | | | | NA07048, NA06993, NA06991, NA12249, |
| | | | | | | | NA10835 |
| | | | 173,669,470 | 173,686,862 | CEU | Hardy-Weinberg | population |
| chr4 | 179,290,796 | 179,297,048 | 179,290,796 | 179,292,684 | YRI | Mendelian inconsistencies | (NA19171, NA19173) |
| | | | 179,292,684 | 179,297,048 | YRI | Mendelian inconsistencies | (NA19171, NA19173) |
| chr4 | 189,917,097 | 189,929,184 | 189,917,097 | 189,929,184 | JCH | Null genotypes | NA18960, NA18965 |
| chr5 | 51,876,796 | 51,890,589 | 51,876,796 | 51,890,589 | YRI | Mendelian inconsistencies | (NA18504, NA18503) |
| chr5 | 109,500,767 | 109,501,608 | 109,500,767 | 109,501,608 | CEU | Mendelian inconsistencies | (NA12005, NA10839) |
| chr5 | 140,256,473 | 140,258,672 | 140,256,473 | 140,258,672 | JCH | Null genotypes | NA18637, NA18990 |
| chr5 | 161,906,500 | 161,932,381 | 161,906,500 | 161,932,381 | CEU | Null genotypes | NA10854, NA12801 |
| chr6 | 7,574,576 | 7,578,276 | 7,574,576 | 7,578,276 | CEU | Mendelian inconsistencies | (NA12875, NA12865) |
| chr6 | 10,579,023 | 10,636,780 | 10,579,023 | 10,609,555 | CEU | Mendelian inconsistencies | (NA12264, NA10863) |
| | | | 10,586,428 | 10,636,780 | CEU | Mendelian inconsistencies | (NA12264, NA10863) |
| chr6 | 19,151,529 | 19,155,942 | 19,151,529 | 19,155,942 | CEU | Null genotypes | NA12872, NA10859 |
| | | | 19,154,609 | 19,155,942 | CEU | Mendelien inconsistencies | (NA12815, NA12802) |
| chr6 | 27,783,269 | 27,784,773 | 27,783,269 | 27,784,773 | YRI | Mendelian inconsistencies | (NA18522, NA18521), (NA19160, NA19161) |
| chr6 | 29,963,788 | 29,971,727 | 29,963,788 | 29,971,727 | YRI | Mendelian inconsistencies | (NA18507, NA18506), (NA19093, NA19094), |
| | | | | | | | (NA19200, NA19202) |
| | | | 29,961,770 | 30,000,151 | JCH | Hardy-Weinberg | population |
| chr6 | 30,032,433 | 30,035,090 | 30,032,433 | 30,035,090 | YRI | Mendelian inconsistencies | (NA19131, NA19132) |
| chr6 | 31,382,098 | 31,387,190 | 31,382,098 | 31,387,190 | YRI | Mendelian inconsistencies | (NA18502, NA18500), (NA19201, NA19202), |
| | | | | | | | (NA19119, NA19120) |
| chr6 | 32,578,057 | 32,582,068 | 32,578,057 | 32,582,068 | YRI | Mendelian inconsistencies | (NA19119, NA19120) |
| | | | 32,569,025 | 32,605,711 | JCH | Hardy-Weinberg | population |
| chr6 | 32,714,840 | 32,718,483 | 32,714,840 | 32,718,483 | YRI | Mendelian inconsistencies | (NA18501, NA18500), (NA18508, NA18506), |
| | | | | | | | (NA18523, NA18521), (NA19192, NA19194) |
| chr6 | 33,985,151 | 33,989,083 | 33,985,151 | 33,989,083 | YRI | Mendelian inconsistencies | (NA18502, NA18500), (NA18855, NA18857), |
| | | | | | | | (NA19092, NA19094), (NA19138, NA19139), |
| | | | | | | | (NA19204, NA19205), (NA19160, NA19161) |
| chr6 | 54,698,192 | 54,707,081 | 54,698,192 | 54,707,081 | CEU | Mendelian inconsistencies | (NA07357, NA07348) |
| | | | 54,698,192 | 54,707,081 | YRI | Mendelian inconsistencies | (NA19209, NA19211), (NA19141, NA19142), |
| | | | | | | | (NA19128, NA19129) |
| chr6 | 77,015,144 | 77,017,575 | 77,015,144 | 77,017,575 | YRI | Mendelian inconsistencies | (NA19128, NA19129) |
| chr6 | 78,968,797 | 78,978,364 | 78,973,263 | 78,978,364 | YRI | Mendelian inconsistencies | (NA18913, NA18914) |
| | | | 78,968,797 | 78,974,994 | YRI | Mendelian inconsistencies | (NA18504, NA18503) |
| chr6 | 78,995,494 | 79,027,965 | 78,998,674 | 79,022,282 | CEU | Mendelian inconsistencies | (NA12144, NA10846), (NA12145, NA10846) |
| | | | 79,021,960 | 79,027,965 | CEU | Mendelian inconsistencies | (NA12003, NA10838), (NA12236, NA10830), |
| | | | | | | | (NA06994, NA07029), (NA07022, NA07019), |
| | | | | | | | (NA12144, NA10846), (NA12145, NA10846), |
| | | | | | | | (NA12249, NA10835) |
| | | | 78,995,494 | 79,021,960 | YRI | Mendelian inconsistencies | (NA18862, NA18863), (NA18913, NA18914) |
| chr6 | 81,280,103 | 81,280,869 | 81,280,103 | 81,280,869 | CEU | Mendelian inconsistencies | (NA07034, NA07048), (NA06993, NA06991) |
| chr6 | 93,571,379 | 93,573,957 | 93,571,379 | 93,573,957 | YRI | Mendelian inconsistencies | (NA19206, NA19208), (NA19116, NA19120) |
| chr6 | 103,784,319 | 103,807,031 | 103,784,319 | 103,807,031 | CEU | Mendelian inconsistencies | (NA12057, NA10851), (NA12145, NA10846), |
| | | | | | | | (NA12891, NA12878), (NA12762, NA12753) |
| | | | 103,787,052 | 103,794,525 | CEU | Mendelian inconsistencies | (NA12004, NA10838), (NA12006, NA10839), |
| | | | | | | | (NA12057, NA10851), (NA11832, NA10855), |
| | | | | | | | (NA12156, NA10831), (NA07000, NA07029), |
| | | | | | | | (NA12716, NA12707), (NA12717, NA12707), |
| | | | | | | | (NA12812, NA12801), (NA12815, NA12802), |
| | | | | | | | (NA12875, NA12865), (NA07055, NA07048), |
| | | | | | | | (NA12249, NA10835) |
| | | | 103,787,052 | 103,807,031 | YRI | Mendelian inconsistencies | (NA19130, NA19132), (NA19131, NA19132) |
| | | | 103,784,468 | 103,799,771 | YRI | Mendelian inconsistencies | (NA18502, NA18500), (NA18852, NA18854), |
| | | | | | | | (NA18862, NA18863), (NA18913, NA18914), |
| | | | | | | | (NA19203, NA19205), (NA19119, NA19120), |
| | | | | | | | (NA19099, NA19100) |
| | | | 103,784,319 | 103,806,709 | CEU | Hardy-Weinberg | population |
| | | | 103,768,785 | 103,807,031 | YRI | Hardy-Weinberg | population |
| | | | 103,768,785 | 103,807,031 | JCH | Hardy-Weinberg | population |
| chr6 | 147,916,589 | 147,923,491 | 147,916,589 | 147,923,491 | CEU | Mendelian inconsistencies | (NA12004, NA10838) |
| chr6 | 148,247,323 | 148,252,401 | 148,247,323 | 148,252,401 | YRI | Mendelian inconsistencies | (NA19092, NA19094) |
| chr7 | 246,846 | 249,228 | 246,846 | 249,228 | JCH | Null genotypes | NA18608, NA18953, NA18948, NA18966, |
| | | | | | | | NA18975, NA19000, NA18978 |
| chr7 | 3,136,864 | 3,171,352 | 3,136,864 | 3,171,352 | YRI | Mendelian inconsistencies | (NA19222, NA19221) |
| chr7 | 7,095,020 | 7,099,452 | 7,095,020 | 7,099,452 | YRI | Mendelian inconsistencies | (NA19099, NA19100) |
| chr7 | 12,848,771 | 12,856,660 | 12,848,771 | 12,856,660 | YRI | Mendelian inconsistencies | (NA18502, NA18500) |
| chr7 | 38,094,780 | 38,112,254 | 38,094,780 | 38,112,254 | CEU | Mendelian inconsistencies | (NA07034, NA07048) |
| chr7 | 61,563,672 | 61,571,616 | 61,563,672 | 61,571,616 | CEU | Null genotypes | NA07055, NA10859 |
| | | | 61,563,672 | 61,571,616 | CEU | Mendelian inconsistencies | (NA12815, NA12802), (NA06985, NA06991) |
| chr7 | 75,886,973 | 75,897,209 | 75,886,973 | 75,897,209 | JCH | Null genotypes | NA18994 |
| chr7 | 75,913,423 | 75,923,509 | 75,913,423 | 75,923,509 | CEU | Null genotypes | NA12005 |
| chr7 | 78,439,557 | 78,445,109 | 78,439,557 | 78,445,109 | YRI | Mendelian inconsistencies | (NA19092, NA19094), (NA19130, NA19132) |
| | | | 78,418,151 | 78,523,025 | JCH | Hardy-Weinberg | population |
| chr7 | 89,422,556 | 89,424,327 | 89,422,556 | 89,424,327 | YRI | Null genotypes | NA18503, NA18523, NA18914, NA19139, |
| | | | | | | | NA19207, NA19116, NA19129, NA19128, |
| | | | | | | | NA19131 |
| | | | 89,422,556 | 89,424,158 | CEU | Null genotypes | NA12056, NA12057, NA10851, NA11830, |
| | | | | | | | NA10856, NA10861, NA07345, NA07348, |
| | | | | | | | NA07000, NA12043, NA12044, NA10857, |
| | | | | | | | NA12145, NA12716, NA12717, NA12707, |
| | | | | | | | NA12812, NA12813, NA12801, NA12815, |
| | | | | | | | NA12864, NA12874, NA12865, NA12760, |
| | | | | | | | NA12753, NA06985, NA12740 |
| chr7 | 90,987,667 | 91,000,865 | 90,987,667 | 91,000,865 | JCH | Null genotypes | NA18964, NA18994 |
| chr7 | 92,092,866 | 92,109,456 | 92,092,866 | 92,101,842 | CEU | Mendelian inconsistencies | (NA11840, NA10854) |
| | | | 92,096,122 | 92,109,456 | CEU | Mendelian inconsistencies | (NA11840, NA10854) |
| chr7 | 92,939,508 | 92,942,586 | 92,939,508 | 92,942,586 | CEU | Null genotypes | NA12056, NA10854, NA12814, NA12864 |
| | | | 92,939,508 | 92,941,787 | CEU | Mendelian inconsistencies | (NA12239, NA10847), (NA12717, NA12707) |
| chr7 | 97,008,440 | 97,012,729 | 97,008,440 | 97,012,729 | CEU | Null genotypes | NA12003, NA12802 |
| | | | 97,008,440 | 97,012,729 | CEU | Mendelian inconsistencies | (NA11993, NA10860), (NA12154, NA10830), |
| | | | | | | | (NA12155, NA10831), (NA11840, NA10854), |
| | | | | | | | (NA12043, NA10857), (NA12872, NA12864) |
| chr7 | 104,193,511 | 104,201,772 | 104,193,511 | 104,201,772 | YRI | Mendelian inconsistencies | (NA19203, NA19205), (NA19222, NA19221) |
| chr7 | 109,002,325 | 109,011,761 | 109,002,325 | 109,007,346 | YRI | Null genotypes | NA18515, NA19202 |
| | | | 109,002,325 | 109,010,654 | JCH | Null genotypes | NA18609, NA18608, NA18566, NA18624, |
| | | | | | | | NA18620, NA18973, NA18964, NA18952, |
| | | | | | | | NA18980, NA18981 |
| | | | 109,003,350 | 109,007,346 | CEU | Null genotypes | NA10851, NA11830, NA11993, NA07357, |
| | | | | | | | NA06994, NA07022, NA07056, NA07019, |
| | | | | | | | NA10846, NA12234, NA12717, NA12707, |
| | | | | | | | NA12801, NA07055, NA10859 |
| | | | 109,002,968 | 109,011,761 | JCH | Null genotypes | NA18609, NA18608, NA18566, NA18624, |
| | | | | | | | NA18620, NA18973, NA18964, NA18952, |
| | | | | | | | NA18980, NA18981 |
| | | | 109,002,968 | 109,011,761 | YRI | Mendelian inconsistencies | (NA18870, NA18872), (NA18913, NA18914), |
| | | | | | | | (NA19116, NA19120), (NA19128, NA19129) |
| | | | 109,002,325 | 109,005,266 | YRI | Mendelian inconsistencies | (NA19116, NA19120), (NA19140, NA19142) |
| | | | 109,003,350 | 109,007,346 | YRI | Mendelian inconsistencies | (NA18870, NA18872), (NA18913, NA18914), |
| | | | | | | | (NA19116, NA19120), (NA19128, NA19129) |
| chr7 | 109,735,375 | 109,745,762 | 109,735,375 | 109,745,762 | JCH | Null genotypes | NA18632, NA18636, NA18593, NA18570, |
| | | | | | | | NA18956, NA18952, NA18991, NA19000 |
| chr7 | 110,398,629 | 110,442,249 | 110,400,767 | 110,439,782 | CEU | Mendelian inconsistencies | (NA11995, NA10861) |
| | | | 110,398,629 | 110,442,249 | CEU | Mendelian inconsistencies | (NA11995, NA10861) |
| chr7 | 115,492,184 | 115,494,416 | 115,492,184 | 115,494,416 | CEU | Mendelian inconsistencies | (NA06994, NA07029), (NA12760, NA12752) |
| chr7 | 117,616,907 | 117,627,348 | 117,616,907 | 117,627,348 | CEU | Mendelian inconsistencies | (NA12813, NA12801) |
| chr7 | 118,709,624 | 118,710,630 | 118,709,624 | 118,710,630 | YRI | Mendelian inconsistencies | (NA18522, NA18521), (NA19172, NA19173) |
| chr7 | 120,111,123 | 120,114,041 | 120,111,123 | 120,114,041 | CEU | Mendelian inconsistencies | (NA12760, NA12752) |
| chr7 | 124,212,558 | 124,213,103 | 124,212,558 | 124,213,103 | YRI | Mendelian inconsistencies | (NA19127, NA19129) |
| chr7 | 125,601,054 | 125,603,762 | 125,601,054 | 125,603,762 | YRI | Null genotypes | NA18504, NA18506, NA18508, NA18860, |
| | | | | | | | NA18859, NA18521, NA18523, NA18522, |
| | | | | | | | NA18870, NA18863, NA18861, NA18913, |
| | | | | | | | NA19103, NA19101, NA19139, NA19138, |
| | | | | | | | NA19205, NA19204, NA19203, NA19208, |
| | | | | | | | NA19206, NA19207, NA19160, NA19222, |
| | | | | | | | NA19116, NA19140, NA19154, NA19152, |
| | | | | | | | NA19145, NA19143, NA19129, NA19127, |
| | | | | | | | NA19128, NA19131, NA19194, NA19193, |
| | | | | | | | NA19192, NA19238 |
| | | | 125,601,054 | 125,603,762 | JCH | Null genotypes | NA18572, NA18547, NA18609, NA18608, |
| | | | | | | | NA18552, NA18611, NA18542, NA18540, |
| | | | | | | | NA18579, NA18635, NA18593, NA18622, |
| | | | | | | | NA18959, NA18972, NA18951, NA18943, |
| | | | | | | | NA18994, NA19007, NA18987, NA18976, |
| | | | | | | | NA18981, NA18971 |
| | | | 125,601,054 | 125,603,762 | CEU | Mendelian inconsistencies | (NA12005, NA10839), (NA11992, NA10860), |
| | | | | | | | (NA12154, NA10830), (NA11840, NA10854), |
| | | | | | | | (NA12145, NA10846), (NA12248, NA10835) |
| chr7 | 133,202,026 | 133,212,391 | 133,209,203 | 133,212,391 | YRI | Null genotypes | NA18504, NA19119 |
| | | | 133,203,070 | 133,212,391 | JCH | Null genotypes | NA18540, NA18624, NA18593, NA18594, |
| | | | | | | | NA18961, NA18940, NA18966 |
| | | | 133,203,070 | 133,212,391 | CEU | Null genotypes | NA12006, NA10839, NA10851, NA11831, |
| | | | | | | | NA11995, NA12155, NA10831, NA07357, |
| | | | | | | | NA07348, NA12264, NA12234, NA10863, |
| | | | | | | | NA12717, NA12707, NA12814, NA12872, |
| | | | | | | | NA12864, NA12763, NA07055, NA12740, |
| | | | | | | | NA12248 |
| | | | 133,209,203 | 133,212,391 | YRI | Mendelian inconsistencies | (NA19209, NA19211), (NA19160, NA19161) |
| | | | 133,202,026 | 133,203,070 | YRI | Mendelian inconsistencies | (NA19137, NA19139) |
| chr7 | 141,179,905 | 141,200,696 | 141,188,320 | 141,199,669 | CEU | Null genotypes | NA07056, NA07019, NA10846, NA06993 |
| | | | 141,179,905 | 141,200,696 | CEU | Null genotypes | NA07056, NA07019, NA10846, NA06993 |
| chr7 | 141,456,537 | 141,472,512 | 141,462,154 | 141,472,285 | YRI | Null genotypes | NA18501, NA18504, NA18506, NA18508, |
| | | | | | | | NA18507, NA18860, NA18858, NA18859, |
| | | | | | | | NA18515, NA18517, NA18516, NA18521, |
| | | | | | | | NA18872, NA18870, NA18871, NA18854, |
| | | | | | | | NA18852, NA18857, NA18855, NA18914, |
| | | | | | | | NA18912, NA18913, NA19093, NA19102, |
| | | | | | | | NA19138, NA19202, NA19200, NA19203, |
| | | | | | | | NA19210, NA19208, NA19160, NA19223, |
| | | | | | | | NA19116, NA19153, NA19145, NA19143, |
| | | | | | | | NA19144, NA19129, NA19128, NA19132, |
| | | | | | | | NA19130, NA19100, NA19099, NA19194, |
| | | | | | | | NA19192, NA19240, NA19239 |
| | | | 141,462,154 | 141,472,285 | JCH | Null genotypes | NA18524, NA18547, NA18550, NA18608, |
| | | | | | | | NA18552, NA18545, NA18558, NA18532, |
| | | | | | | | NA18561, NA18537, NA18603, NA18563, |
| | | | | | | | NA18579, NA18633, NA18635, NA18593, |
| | | | | | | | NA18620, NA18621, NA18594, NA18622, |
| | | | | | | | NA18573, NA18637, NA18526, NA18942, |
| | | | | | | | NA18953, NA18968, NA18961, NA18965, |
| | | | | | | | NA18973, NA18964, NA18956, NA18947, |
| | | | | | | | NA18944, NA18945, NA18992, NA18997, |
| | | | | | | | NA18998, NA19000, NA18987, NA18967, |
| | | | | | | | NA18976, NA18978, NA19003 |
| | | | 141,462,154 | 141,472,512 | CEU | Null genotypes | NA12004, NA10838, NA11832, NA10855, |
| | | | | | | | NA11995, NA12154, NA12156, NA07357, |
| | | | | | | | NA12044, NA12144, NA12892, NA12815, |
| | | | | | | | NA12802, NA12872, NA12864, NA12762, |
| | | | | | | | NA07034, NA07055, NA07048 |
| | | | 141,460,703 | 141,472,285 | JCH | Null genotypes | NA18524, NA18547, NA18550, NA18608, |
| | | | | | | | NA18552, NA18545, NA18558, NA18532, |
| | | | | | | | NA18561, NA18537, NA18603, NA18563, |
| | | | | | | | NA18579, NA18633, NA18635, NA18593, |
| | | | | | | | NA18620, NA18621, NA18594, NA18622, |
| | | | | | | | NA18573, NA18637, NA18526, NA18942, |
| | | | | | | | NA18953, NA18968, NA18961, NA18965, |
| | | | | | | | NA18973, NA18964, NA18956, NA18947, |
| | | | | | | | NA18944, NA18945, NA18992, NA18997, |
| | | | | | | | NA18998, NA19000, NA18990, NA18987, |
| | | | | | | | NA18967, NA18976, NA18978, NA19003 |
| | | | 141,456,537 | 141,472,512 | CEU | Null genotypes | NA12004, NA10838, NA10851, NA11832, |
| | | | | | | | NA10855, NA11995, NA12154, NA12156, |
| | | | | | | | NA07357, NA12044, NA12144, NA12892, |
| | | | | | | | NA12815, NA12802, NA12872, NA12864, |
| | | | | | | | NA12761, NA12762, NA07034, NA07055, |
| | | | | | | | NA07048 |
| | | | 141,456,537 | 141,472,285 | YRI | Null genotypes | NA18500, NA18501, NA18504, NA18506, |
| | | | | | | | NA18508, NA18507, NA18860, NA18858, |
| | | | | | | | NA18859, NA18515, NA18517, NA18516, |
| | | | | | | | NA18521, NA18872, NA18870, NA18871, |
| | | | | | | | NA18854, NA18852, NA18857, NA18855, |
| | | | | | | | NA18914, NA18912, NA18913, NA19093, |
| | | | | | | | NA19102, NA19138, NA19202, NA19200, |
| | | | | | | | NA19203, NA19210, NA19208, NA19160, |
| | | | | | | | NA19223, NA19116, NA19153, NA19145, |
| | | | | | | | NA19143, NA19144, NA19129, NA19128, |
| | | | | | | | NA19132, NA19130, NA19100, NA19099, |
| | | | | | | | NA19194, NA19192, NA19240, NA19239 |
| | | | 141,469,162 | 141,470,799 | CEU | Mendelian inconsistencies | (NA12006, NA10839), (NA12057, NA10851), |
| | | | | | | | (NA11992, NA10860) |
| chr7 | 141,657,311 | 141,669,388 | 141,657,311 | 141,669,388 | CEU | Mendelian inconsistencies | (NA12813, NA12801) |
| chr7 | 141,730,581 | 141,765,123 | 141,730,581 | 141,765,123 | CEU | Mendelian inconsistencies | (NA12144, NA10846) |
| | | | 141,730,581 | 141,765,123 | CEU | Mendelian inconsistencies | (NA12144, NA10846) |
| chr7 | 141,921,685 | 141,931,471 | 141,922,974 | 141,927,931 | JCH | Null genotypes | NA18524, NA18547, NA18609, NA18550, |
| | | | | | | | NA18552, NA18611, NA18555, NA18529, |
| | | | | | | | NA18532, NA18561, NA18537, NA18603, |
| | | | | | | | NA18605, NA18582, NA18635, NA18636, |
| | | | | | | | NA18577, NA18571, NA18620, NA18621, |
| | | | | | | | NA18622, NA18573, NA18623, NA18637, |
| | | | | | | | NA18959, NA18960, NA18961, NA18973, |
| | | | | | | | NA18956, NA18940, NA18943, NA18944, |
| | | | | | | | NA18945, NA18949, NA18952, NA18966, |
| | | | | | | | NA18975, NA18992, NA18998, NA19007, |
| | | | | | | | NA18990, NA18978, NA18970, NA18980, |
| | | | | | | | NA18995, NA18974, NA19003 |
| | | | 141,921,685 | 141,931,471 | CEU | Null genotypes | NA10851, NA11829, NA11993, NA07345, |
| | | | | | | | NA10846, NA12264, NA12716, NA12812, |
| | | | | | | | NA12761, NA06991 |
| | | | 141,922,974 | 141,931,471 | JCH | Null genotypes | NA18524, NA18547, NA18609, NA18550, |
| | | | | | | | NA18552, NA18611, NA18555, NA18529, |
| | | | | | | | NA18532, NA18561, NA18537, NA18605, |
| | | | | | | | NA18582, NA18635, NA18636, NA18577, |
| | | | | | | | NA18571, NA18620, NA18621, NA18622, |
| | | | | | | | NA18573, NA18623, NA18637, NA18959, |
| | | | | | | | NA18960, NA18961, NA18940, NA18944, |
| | | | | | | | NA18949, NA18952, NA18992, NA18998, |
| | | | | | | | NA19007, NA18978, NA18970, NA18980, |
| | | | | | | | NA18995, NA19003 |
| chr7 | 149,625,782 | 149,631,027 | 149,625,782 | 149,631,027 | CEU | Mendelian inconsistencies | (NA11831, NA10855) |
| chr7 | 157,855,041 | 157,857,130 | 157,855,041 | 157,857,130 | JCH | Null genotypes | NA18566, NA18593, NA18947 |
| chr8 | 587,487 | 588,391 | 587,487 | 588,391 | JCH | Null genotypes | NA18547, NA18609, NA18550, NA18608, |
| | | | | | | | NA18611, NA18632, NA18635, NA18593, |
| | | | | | | | NA18576, NA18612, NA18594, NA18622, |
| | | | | | | | NA18953, NA18960, NA18961, NA18972, |
| | | | | | | | NA18956, NA18949, NA18966, NA18975, |
| | | | | | | | NA18998, NA19000, NA18987, NA18976, |
| | | | | | | | NA18995, NA18974 |
| | | | 534,755 | 588,391 | CEU | Hardy-Weinberg | population |
| chr8 | 2,066,222 | 2,067,696 | 2,066,222 | 2,067,696 | CEU | Mendelian inconsistencies | (NA12872, NA12864) |
| | | | 2,066,222 | 2,075,642 | YRI | Hardy-Weinberg | population |
| chr8 | 2,242,110 | 2,250,519 | 2,242,110 | 2,250,519 | YRI | Null genotypes | NA18500, NA18501, NA19103, NA19101, |
| | | | | | | | NA19137, NA19203, NA19209, NA19127, |
| | | | | | | | NA19130 |
| | | | 2,242,110 | 2,243,578 | CEU | Mendelian inconsistencies | (NA07000, NA07029) |
| | | | 2,242,110 | 2,244,333 | YRI | Mendelian inconsistencies | (NA18504, NA18503), (NA19172, NA19173) |
| chr8 | 3,987,468 | 3,992,429 | 3,987,468 | 3,992,429 | JCH | Null genotypes | NA19007 |
| chr8 | 4,039,867 | 4,040,487 | 4,039,867 | 4,040,487 | JCH | Null genotypes | NA19007 |
| chr8 | 4,150,121 | 4,158,194 | 4,150,121 | 4,158,194 | JCH | Null genotypes | NA19007 |
| chr8 | 4,173,158 | 4,183,588 | 4,173,158 | 4,183,588 | JCH | Null genotypes | NA19007 |
| chr8 | 4,576,576 | 4,586,136 | 4,576,576 | 4,586,136 | JCH | Null genotypes | NA19007 |
| chr8 | 4,619,513 | 4,691,949 | 4,619,513 | 4,691,949 | YRI | Mendelian inconsistencies | (NA18912, NA18914) |
| chr8 | 4,708,927 | 4,717,662 | 4,708,927 | 4,717,662 | YRI | Mendelian inconsistencies | (NA19160, NA19161) |
| chr8 | 5,065,307 | 5,076,194 | 5,065,307 | 5,076,194 | JCH | Null genotypes | NA19007 |
| chr8 | 5,340,694 | 5,350,447 | 5,340,694 | 5,350,447 | JCH | Null genotypes | NA19007 |
| chr8 | 5,587,999 | 5,590,045 | 5,587,999 | 5,590,045 | CEU | Mendelian inconsistencies | (NA11882, NA10859) |
| chr8 | 5,638,409 | 5,641,012 | 5,638,409 | 5,641,012 | JCH | Null genotypes | NA19007 |
| chr8 | 6,052,575 | 6,056,851 | 6,052,575 | 6,056,851 | YRI | Mendelian inconsistencies | (NA19223, NA19221) |
| chr8 | 6,108,358 | 6,147,262 | 6,108,358 | 6,147,262 | JCH | Null genotypes | NA18537 |
| chr8 | 6,810,705 | 6,811,452 | 6,810,705 | 6,811,452 | CEU | Mendelian inconsistencies | (NA12813, NA12801) |
| chr8 | 7,201,387 | 7,206,953 | 7,201,387 | 7,206,953 | CEU | Mendelian inconsistencies | (NA06994, NA07029) |
| | | | 7,201,387 | 7,206,953 | YRI | Mendelian inconsistencies | (NA18871, NA18872), (NA18862, NA18863), |
| | | | | | | | (NA19138, NA19139), (NA19140, NA19142), |
| | | | | | | | (NA19239, NA19240) |
| chr8 | 7,814,659 | 7,824,363 | 7,814,659 | 7,824,363 | CEU | Mendelian inconsistencies | (NA11839, NA10854), (NA12234, NA10863), |
| | | | | | | | (NA12813, NA12801) |
| chr8 | 9,537,015 | 9,537,654 | 9,537,015 | 9,537,654 | JCH | Null genotypes | NA18558, NA18562, NA18959, NA18944, |
| | | | | | | | NA18949, NA18981 |
| chr8 | 12,242,025 | 12,257,919 | 12,242,025 | 12,257,919 | CEU | Mendelian inconsistencies | (NA07034, NA07048), (NA06993, NA06991) |
| | | | 12,242,025 | 12,257,919 | CEU | Mendelian inconsistencies | (NA07034, NA07048), (NA06993, NA06991) |
| chr8 | 12,556,004 | 12,565,109 | 12,556,004 | 12,565,109 | JCH | Null genotypes | NA18561, NA18621 |
| chr8 | 13,625,501 | 13,659,196 | 13,625,501 | 13,659,196 | YRI | Null genotypes | NA19205 |
| | | | 13,637,016 | 13,656,796 | YRI | Mendelian inconsistencies | (NA18505, NA18503), (NA18522, NA18521), |
| | | | | | | | (NA19092, NA19094) |
| | | | 13,625,501 | 13,630,545 | YRI | Mendelian inconsistencies | (NA18505, NA18503) |
| | | | 13,625,501 | 13,659,196 | YRI | Hardy-Weinberg | population |
| chr8 | 14,590,534 | 14,596,692 | 14,590,534 | 14,596,692 | YRI | Null genotypes | NA18507, NA18863, NA18862, NA18914, |
| | | | | | | | NA18912, NA18913, NA19102, NA19171, |
| | | | | | | | NA19152, NA19131, NA19240, NA19238, |
| | | | | | | | NA19239 |
| chr8 | 14,647,130 | 15,392,548 | 14,650,691 | 15,336,034 | CEU | Mendelian inconsistencies | (NA12234, NA10863) |
| | | | 14,978,429 | 15,392,548 | CEU | Mendelian inconsistencies | (NA12234, NA10863) |
| | | | 14,647,130 | 15,337,510 | CEU | Mendelian inconsistencies | (NA12234, NA10863) |
| chr8 | 15,622,969 | 15,670,924 | 15,643,763 | 15,646,128 | CEU | Mendelian inconsistencies | (NA12234, NA10863) |
| | | | 15,622,969 | 15,670,924 | CEU | Mendelian inconsistencies | (NA12234, NA10863) |
| | | | 15,627,172 | 15,632,885 | CEU | Mendelian inconsistencies | (NA12234, NA10863) |
| chr8 | 16,211,924 | 16,216,726 | 16,212,027 | 16,216,726 | YRI | Null genotypes | NA18505, NA18508, NA18515, NA18517, |
| | | | | | | | NA18516, NA18854, NA18852, NA19201, |
| | | | | | | | NA19204, NA19211, NA19209, NA19208, |
| | | | | | | | NA19160, NA19153, NA19129, NA19128, |
| | | | | | | | NA19130 |
| | | | 16,211,924 | 16,216,726 | JCH | Null genotypes | NA18612, NA18956, NA18975, NA18992, |
| | | | | | | | NA18998, NA18990 |
| | | | 16,211,924 | 16,216,726 | CEU | Mendelian inconsistencies | (NA12760, NA12752), (NA12248, NA10835) |
| | | | 16,211,924 | 16,215,120 | YRI | Mendelian inconsistencies | (NA18522, NA18521), (NA19192, NA19194) |
| | | | 16,211,924 | 16,216,201 | YRI | Hardy-Weinberg | population |
| chr8 | 16,277,375 | 16,280,833 | 16,277,375 | 16,280,833 | YRI | Mendelian inconsistencies | (NA19204, NA19205) |
| chr8 | 20,003,375 | 20,033,444 | 20,003,375 | 20,033,444 | JCH | Null genotypes | NA18997 |
| chr8 | 24,996,369 | 25,011,464 | 24,996,369 | 25,011,464 | YRI | Null genotypes | NA18506 |
| | | | 24,998,571 | 25,011,464 | JCH | Null genotypes | NA18999 |
| | | | 25,007,859 | 25,011,464 | CEU | Mendelian inconsistencies | (NA11995, NA10861), (NA12892, NA12878) |
| | | | 25,009,265 | 25,011,464 | CEU | Mendelian inconsistencies | (NA11995, NA10861), (NA07357, NA07348), |
| | | | | | | | (NA07345, NA07348), (NA12892, NA12878) |
| | | | 25,001,043 | 25,006,243 | CEU | Mendelian inconsistencies | (NA12004, NA10838), (NA12005, NA10839), |
| | | | | | | | (NA12006, NA10839), (NA11995, NA10861), |
| | | | | | | | (NA07345, NA07348), (NA06994, NA07029), |
| | | | | | | | (NA12144, NA10846), (NA12145, NA10846), |
| | | | | | | | (NA12716, NA12707), (NA12812, NA12801) |
| | | | 24,996,369 | 25,011,464 | YRI | Mendelian inconsistencies | (NA19098, NA19100), (NA19239, NA19240) |
| | | | 25,001,043 | 25,011,464 | CEU | Hardy-Weinberg | population |
| chr8 | 25,433,955 | 25,436,600 | 25,434,487 | 25,436,600 | CEU | Mendelian inconsistencies | (NA11994, NA10861), (NA12144, NA10846), |
| | | | | | | | (NA06993, NA06991) |
| | | | 25,433,955 | 25,434,487 | YRI | Mendelian inconsistencies | (NA19127, NA19129) |
| chr8 | 38,125,501 | 38,127,583 | 38,125,501 | 38,127,583 | CEU | Null genotypes | NA10856, NA11882 |
| chr8 | 39,250,107 | 39,404,547 | 39,250,107 | 39,397,764 | CEU | Null genotypes | NA10839, NA11829, NA11992, NA12154, |
| | | | | | | | NA12156, NA10831, NA12144, NA12239, |
| | | | | | | | NA12264, NA12716, NA12891, NA12813, |
| | | | | | | | NA12814, NA12873, NA12864, NA12875, |
| | | | | | | | NA12249, NA10835 |
| | | | 39,250,107 | 39,404,547 | JCH | Null genotypes | NA18524, NA18526, NA18942, NA18967 |
| | | | 39,268,398 | 39,389,812 | CEU | Null genotypes | NA10839, NA11829, NA11992, NA12154, |
| | | | | | | | NA12156, NA10831, NA12144, NA12239, |
| | | | | | | | NA12264, NA12716, NA12891, NA12813, |
| | | | | | | | NA12814, NA12873, NA12864, NA12875, |
| | | | | | | | NA12249, NA10835 |
| | | | 39,386,491 | 39,390,862 | CEU | Mendelian inconsistencies | (NA12004, NA10838), (NA11831, NA10855), |
| | | | | | | | (NA12750, NA12740) |
| | | | 39,271,742 | 39,390,071 | CEU | Mendelian inconsistencies | (NA12005, NA10839), (NA12006, NA10839), |
| | | | | | | | (NA12057, NA10851), (NA11829, NA10856), |
| | | | | | | | (NA11831, NA10855), (NA11992, NA10860), |
| | | | | | | | (NA12155, NA10831), (NA12144, NA10846), |
| | | | | | | | (NA12239, NA10847), (NA12264, NA10863), |
| | | | | | | | (NA12813, NA12801), (NA12814, NA12802), |
| | | | | | | | (NA12872, NA12864), (NA07055, NA07048), |
| | | | | | | | (NA12750, NA12740), (NA12248, NA10835) |
| | | | 39,304,599 | 39,320,127 | CEU | Mendelian inconsistencies | (NA12005, NA10839), (NA12006, NA10839), |
| | | | | | | | (NA11829, NA10856), (NA11831, NA10855), |
| | | | | | | | (NA12155, NA10831), (NA12813, NA12801), |
| | | | | | | | (NA07055, NA07048), (NA12750, NA12740), |
| | | | | | | | (NA12248, NA10835) |
| | | | 39,292,394 | 39,316,841 | CEU | Mendelian inconsistencies | (NA11831, NA10855), (NA11839, NA10854), |
| | | | | | | | (NA12813, NA12801), (NA12248, NA10835) |
| | | | 39,291,521 | 39,299,946 | CEU | Mendelian inconsistencies | (NA11831, NA10855), (NA11839, NA10854) |
| | | | 39,304,817 | 39,319,367 | CEU | Mendelian inconsistencies | (NA11839, NA10854) |
| | | | 39,250,107 | 39,255,036 | CEU | Mendelian inconsistencies | (NA11831, NA10855) |
| | | | 39,334,593 | 39,387,126 | CEU | Mendelian inconsistencies | (NA06985, NA06991) |
| | | | 39,271,742 | 39,390,862 | YRI | Mendelian inconsistencies | (NA19203, NA19205), (NA19207, NA19208) |
| | | | 39,250,107 | 39,326,538 | YRI | Mendelian inconsistencies | (NA18858, NA18860) |
| | | | 39,268,256 | 39,296,584 | YRI | Mendelian inconsistencies | (NA19203, NA19205) |
| | | | 39,387,126 | 39,404,547 | YRI | Mendelian inconsistencies | (NA19203, NA19205) |
| | | | 39,250,107 | 39,411,423 | CEU | Hardy-Weinberg | population |
| | | | 39,255,036 | 39,325,989 | CEU | Hardy-Weinberg | population |
| chr8 | 40,201,780 | 40,206,917 | 40,201,780 | 40,206,917 | CEU | Mendelian inconsistencies | (NA12156, NA10831), (NA12239, NA10847) |
| chr8 | 41,124,713 | 41,125,490 | 41,124,713 | 41,125,490 | JCH | Null genotypes | NA18632 |
| chr8 | 51,082,185 | 51,083,978 | 51,082,185 | 51,083,978 | YRI | Mendelian inconsistencies | (NA19201, NA19202) |
| chr8 | 54,202,942 | 54,211,318 | 54,202,942 | 54,211,318 | YRI | Null genotypes | NA18860, NA18854, NA19103, NA19132, |
| | | | | | | | NA19100 |
| chr8 | 55,414,544 | 55,423,847 | 55,414,544 | 55,423,847 | YRI | Null genotypes | NA19172, NA19128 |
| chr8 | 57,668,667 | 57,679,183 | 57,668,667 | 57,679,183 | YRI | Null genotypes | NA18500 |
| chr8 | 58,413,964 | 58,415,083 | 58,413,964 | 58,415,083 | YRI | Mendelian inconsistencies | (NA19127, NA19129) |
| chr8 | 59,355,794 | 59,368,409 | 59,355,794 | 59,368,409 | JCH | Null genotypes | NA18940, NA19003 |
| chr8 | 61,051,468 | 61,055,811 | 61,051,468 | 61,055,811 | YRI | Null genotypes | NA18516 |
| chr8 | 64,899,411 | 64,900,642 | 64,899,411 | 64,900,642 | YRI | Null genotypes | NA19132, NA19131 |
| chr8 | 65,165,838 | 65,167,766 | 65,165,838 | 65,167,766 | CEU | Null genotypes | NA11994, NA10861 |
| chr8 | 68,039,962 | 68,043,387 | 68,039,962 | 68,043,387 | YRI | Null genotypes | NA18861, NA19211, NA19120, NA19116, |
| | | | | | | | NA19119, NA19141, NA19130, NA19100 |
| chr8 | 70,997,239 | 71,011,390 | 70,997,239 | 71,011,390 | YRI | Mendelian inconsistencies | (NA18913, NA18914) |
| chr8 | 73,673,604 | 73,685,915 | 73,673,604 | 73,685,915 | CEU | Null genotypes | NA12873 |
| chr8 | 82,429,915 | 82,431,085 | 82,429,915 | 82,431,085 | YRI | Null genotypes | NA19205, NA19204 |
| chr8 | 85,311,119 | 85,315,959 | 85,311,119 | 85,315,959 | CEU | Mendelian inconsistencies | (NA12057, NA10851), (NA12872, NA12864) |
| chr8 | 90,287,377 | 90,293,533 | 90,287,377 | 90,292,349 | YRI | Null genotypes | NA19173, NA19171 |
| | | | 90,287,607 | 90,293,533 | YRI | Mendelian inconsistencies | (NA19171, NA19173) |
| chr8 | 91,140,357 | 91,143,301 | 91,140,357 | 91,143,301 | JCH | Null genotypes | NA18971 |
| chr8 | 93,654,573 | 93,663,141 | 93,654,573 | 93,663,141 | CEU | Null genotypes | NA10860, NA07056, NA12892, NA12878 |
| chr8 | 95,515,277 | 95,528,026 | 95,515,277 | 95,528,026 | YRI | Null genotypes | NA19116 |
| chr8 | 101,615,219 | 101,615,751 | 101,615,219 | 101,615,751 | CEU | Null genotypes | NA06985 |
| chr8 | 103,010,682 | 103,011,802 | 103,010,682 | 103,011,802 | YRI | Null genotypes | NA18860, NA18858, NA18522, NA19139, |
| | | | | | | | NA19138 |
| chr8 | 107,045,009 | 107,047,083 | 107,045,009 | 107,047,083 | CEU | Mendelian inconsistencies | (NA12875, NA12865) |
| chr8 | 107,817,446 | 107,818,580 | 107,817,446 | 107,818,580 | YRI | Mendelian inconsistencies | (NA19203, NA19205) |
| chr8 | 115,126,252 | 115,130,784 | 115,126,252 | 115,130,784 | YRI | Null genotypes | NA19131, NA19130 |
| chr8 | 115,591,865 | 115,599,078 | 115,591,865 | 115,599,078 | JCH | Null genotypes | NA18636, NA18612, NA18956, NA18947, |
| | | | | | | | NA18948 |
| | | | 115,540,689 | 115,611,467 | JCH | Hardy-Weinberg | population |
| chr8 | 121,829,317 | 121,829,933 | 121,829,317 | 121,829,933 | YRI | Mendelian inconsistencies | (NA19127, NA19129) |
| chr8 | 123,027,122 | 123,034,224 | 123,027,122 | 123,034,224 | YRI | Null genotypes | NA18516 |
| chr8 | 137,811,456 | 137,822,815 | 137,811,456 | 137,822,815 | CEU | Mendelian inconsistencies | (NA12249, NA10835) |
| chr8 | 141,973,953 | 141,975,249 | 141,973,953 | 141,975,249 | YRI | Null genotypes | NA19116 |
| chr9 | 205,917 | 238,749 | 205,917 | 238,749 | JCH | Null genotypes | NA18576 |
| chr9 | 581,040 | 598,622 | 581,040 | 592,986 | YRI | Null genotypes | NA18862, NA19201 |
| | | | 582,004 | 598,622 | YRI | Null genotypes | NA18862 |
| chr9 | 665,600 | 685,607 | 665,600 | 685,607 | YRI | Mendelian inconsistencies | (NA18859, NA18860) |
| chr9 | 1,500,299 | 1,516,383 | 1,500,299 | 1,516,383 | YRI | Mendelian inconsistencies | (NA18870, NA18872) |
| chr9 | 4,006,814 | 4,009,224 | 4,006,814 | 4,009,224 | YRI | Null genotypes | NA19093, NA19092 |
| chr9 | 5,102,519 | 5,103,577 | 5,102,519 | 5,103,577 | YRI | Null genotypes | NA18505, NA19093 |
| chr9 | 9,791,153 | 9,794,313 | 9,791,153 | 9,793,954 | CEU | Mendelian inconsistencies | (NA11840, NA10854) |
| | | | 9,791,153 | 9,794,313 | CEU | Mendelian inconsistencies | (NA11840, NA10854) |
| chr9 | 10,536,814 | 10,572,586 | 10,536,814 | 10,572,586 | CEU | Mendelian inconsistencies | (NA12760, NA12752) |
| chr9 | 11,881,381 | 11,883,047 | 11,881,381 | 11,883,047 | YRI | Null genotypes | NA18859, NA19221, NA19222 |
| chr9 | 11,903,287 | 11,978,036 | 11,903,287 | 11,978,036 | YRI | Mendelian inconsistencies | (NA19159, NA19161) |
| chr9 | 21,180,827 | 21,189,680 | 21,180,827 | 21,189,680 | YRI | Null genotypes | NA19131 |
| chr9 | 21,275,389 | 21,296,318 | 21,278,018 | 21,285,828 | YRI | Null genotypes | NA18914, NA18912, NA19208 |
| | | | 21,275,389 | 21,284,921 | YRI | Null genotypes | NA18914, NA18912 |
| | | | 21,283,631 | 21,296,318 | YRI | Mendelian inconsistencies | (NA18912, NA18914) |
| chr9 | 23,759,754 | 23,765,449 | 23,759,754 | 23,765,449 | CEU | Mendelian inconsistencies | (NA06994, NA07029) |
| | | | 23,759,754 | 23,765,449 | YRI | Mendelian inconsistencies | (NA19144, NA19145) |
| chr9 | 24,457,698 | 24,484,302 | 24,457,698 | 24,484,302 | CEU | Null genotypes | NA11881 |
| chr9 | 29,914,682 | 29,976,250 | 29,933,652 | 29,965,253 | CEU | Null genotypes | NA07048 |
| | | | 29,929,173 | 29,948,595 | CEU | Mendelian inconsistencies | (NA07055, NA07048) |
| | | | 29,914,682 | 29,976,250 | CEU | Mendelian inconsistencies | (NA07055, NA07048) |
| chr9 | 32,991,449 | 33,014,917 | 32,991,449 | 33,014,917 | CEU | Null genotypes | NA12057, NA07357 |
| chr9 | 36,872,141 | 36,890,817 | 36,872,141 | 36,890,817 | YRI | Null genotypes | NA18500, NA19207 |
| chr9 | 38,509,912 | 38,524,245 | 38,509,912 | 38,524,245 | CEU | Null genotypes | NA12872 |
| chr9 | 43,348,537 | 43,374,214 | 43,348,537 | 43,374,214 | YRI | Null genotypes | NA18500, NA19207 |
| chr9 | 62,545,375 | 62,570,383 | 62,545,375 | 62,570,383 | YRI | Null genotypes | NA18500, NA19207 |
| chr9 | 67,563,748 | 67,566,196 | 67,563,748 | 67,566,196 | JCH | Null genotypes | NA18953 |
| chr9 | 81,945,705 | 81,999,325 | 81,945,705 | 81,999,325 | CEU | Null genotypes | NA12872 |
| chr9 | 89,737,468 | 89,742,167 | 89,737,468 | 89,742,167 | CEU | Null genotypes | NA12760 |
| chr9 | 100,233,285 | 100,243,634 | 100,233,285 | 100,243,634 | JCH | Null genotypes | NA18572 |
| chr9 | 102,745,808 | 102,746,309 | 102,745,808 | 102,746,309 | YRI | Mendelian inconsistencies | (NA19204, NA19205), (NA19152, NA19154) |
| chr9 | 109,440,549 | 109,445,124 | 109,440,549 | 109,445,124 | YRI | Null genotypes | NA19159 |
| chr9 | 124,896,840 | 124,898,353 | 124,896,840 | 124,898,353 | JCH | Null genotypes | NA18532, NA18582, NA18952, NA18998, |
| | | | | | | | NA18970, NA18995 |
| chr9 | 129,658,718 | 129,724,341 | 129,658,718 | 129,724,341 | JCH | Null genotypes | NA18967 |
| chr9 | 133,660,972 | 133,664,954 | 133,660,972 | 133,664,954 | JCH | Null genotypes | NA18526, NA18999 |
| chr9 | 136,191,747 | 136,197,190 | 136,191,747 | 136,197,190 | JCH | Null genotypes | NA18540, NA18960 |
| chr9 | 136,222,560 | 136,230,195 | 136,222,560 | 136,230,195 | CEU | Null genotypes | NA12814 |
| chrX | 3,700,009 | 3,708,183 | 3,700,009 | 3,708,183 | YRI | Null genotypes | NA19205, NA19119 |
| chrX | 5,225,704 | 5,245,361 | 5,225,704 | 5,245,361 | YRI | Mendelian inconsistencies | (NA18501, NA18500), (NA18504, NA18503) |
| chrX | 6,811,025 | 6,978,830 | 6,844,853 | 6,950,030 | CEU | Null genotypes | NA10854, NA12248 |
| | | | 6,811,025 | 6,978,830 | CEU | Null genotypes | NA12248 |
| chrX | 7,583,812 | 7,590,193 | 7,583,812 | 7,590,193 | YRI | Null genotypes | NA18856 |
| chrX | 11,381,612 | 11,410,075 | 11,381,612 | 11,410,075 | CEU | Mendelian inconsistencies | (NA12003, NA10838), (NA12005, NA10839) |
| chrX | 15,834,905 | 15,839,616 | 15,834,905 | 15,839,616 | YRI | Null genotypes | NA18854, NA19161 |
| chrX | 15,964,777 | 15,971,948 | 15,964,777 | 15,971,948 | YRI | Null genotypes | NA19103, NA19141, NA19154, NA19128 |
| chrX | 27,404,104 | 27,412,916 | 27,404,104 | 27,412,916 | JCH | Null genotypes | NA18972 |
| | | | 27,395,883 | 27,463,391 | YRI | Hardy-Weinberg | population |
| chrX | 31,917,231 | 31,922,836 | 31,917,231 | 31,922,836 | YRI | Mendelian inconsistencies | (NA18501, NA18500), (NA18504, NA18503) |
| chrX | 33,482,313 | 33,483,048 | 33,482,313 | 33,483,048 | YRI | Null genotypes | NA19206, NA19141 |
| chrX | 46,532,540 | 46,534,975 | 46,532,540 | 46,534,975 | YRI | Mendelian inconsistencies | (NA18502, NA18500), (NA19223, NA19221) |
| chrX | 46,929,298 | 47,028,433 | 46,929,298 | 47,028,433 | YRI | Null genotypes | NA18856 |
| chrX | 55,918,254 | 55,922,827 | 55,918,254 | 55,922,827 | YRI | Null genotypes | NA18504, NA19138 |
| chrX | 57,162,074 | 57,167,037 | 57,162,074 | 57,167,037 | JCH | Null genotypes | NA18632 |
| chrX | 57,430,265 | 57,450,038 | 57,430,265 | 57,450,038 | YRI | Null genotypes | NA18501 |
| chrX | 62,874,612 | 62,922,110 | 62,874,612 | 62,922,110 | JCH | Null genotypes | NA18995 |
| chrX | 65,105,136 | 65,531,010 | 65,105,136 | 65,531,010 | YRI | Mendelian inconsistencies | (NA18501, NA18500), (NA18502, NA18500), |
| | | | | | | | (NA18504, NA18503) |
| chrX | 74,996,468 | 75,010,864 | 74,996,468 | 75,010,864 | CEU | Null genotypes | NA12864 |
| chrx | 77,858,920 | 77,859,903 | 77,858,920 | 77,859,903 | CEU | Null genotypes | NA10856, NA10854 |
| chrX | 80,152,214 | 80,167,843 | 80,152,214 | 80,167,843 | YRI | Null genotypes | NA18504, NA19103 |
| chrX | 83,958,997 | 83,971,986 | 83,958,997 | 83,971,986 | JCH | Null genotypes | NA18532, NA18540 |
| chrX | 84,097,369 | 84,104,170 | 84,097,369 | 84,104,170 | JCH | Null genotypes | NA18532, NA18540 |
| chrX | 88,669,195 | 88,680,388 | 88,669,195 | 88,680,388 | CEU | Null genotypes | NA12003, NA12761 |
| | | | 88,670,011 | 88,673,690 | CEU | Null genotypes | NA12003 |
| chrX | 89,743,313 | 89,750,185 | 89,743,313 | 89,750,185 | YRI | Null genotypes | NA18871, NA19138, NA19145 |
| | | | 89,743,313 | 89,750,185 | YRI | Mendelian inconsistencies | (NA19093, NA19094) |
| chrX | 91,086,005 | 91,109,766 | 91,086,005 | 91,109,766 | YRI | Null genotypes | NA19200, NA19159 |
| chrX | 92,173,430 | 92,175,756 | 92,173,430 | 92,175,756 | CEU | Null genotypes | NA10838 |
| | | | 92,173,430 | 92,175,756 | YRI | Null genotypes | NA18503, NA18506, NA18856 |
| | | | 92,173,430 | 92,175,756 | JCH | Null genotypes | NA18945 |
| chrX | 95,742,982 | 95,757,258 | 95,742,982 | 95,757,258 | JCH | Null genotypes | NA18611 |
| chrX | 107,659,218 | 107,737,812 | 107,662,335 | 107,675,738 | YRI | Null genotypes | NA19223, NA19153 |
| | | | 107,659,218 | 107,737,812 | YRI | Mendelian inconsistencies | (NA18501, NA18500), (NA18504, NA18503) |
| chrX | 108,399,188 | 108,404,026 | 108,399,188 | 108,404,026 | YRI | Null genotypes | NA19211 |
| chrX | 108,703,176 | 108,705,130 | 108,703,176 | 108,705,130 | YRI | Null genotypes | NA18501, NA19160, NA19127 |
| chrX | 110,928,593 | 110,929,387 | 110,928,593 | 110,929,387 | JCH | Null genotypes | NA18608, NA18633 |
| chrX | 114,373,214 | 114,373,881 | 114,373,214 | 114,373,881 | JCH | Null genotypes | NA18540, NA18943 |
| chrX | 119,102,962 | 119,108,525 | 119,102,962 | 119,108,525 | JCH | Null genotypes | NA18637 |
| chrX | 121,803,546 | 121,804,252 | 121,803,546 | 121,804,252 | JCH | Null genotypes | NA18540, NA18995 |
| chrX | 140,165,208 | 140,166,897 | 140,165,208 | 140,166,897 | JCH | Null genotypes | NA18562, NA18563 |
| chrX | 144,847,796 | 144,850,328 | 144,847,796 | 144,850,328 | CEU | Null genotypes | NA12872 |
| chrX | 145,514,196 | 145,515,614 | 145,514,196 | 145,515,614 | YRI | Null genotypes | NA18522, NA19153 |
| chrX | 147,101,408 | 147,102,204 | 147,101,408 | 147,102,204 | YRI | Null genotypes | NA18500, NA19098 |
| chrX | 147,351,039 | 147,356,073 | 147,351,039 | 147,356,073 | YRI | Null genotypes | NA18506, NA18522, NA19173, NA19161, |
| | | | | | | | NA19144 |
| chrX | 153,206,494 | 153,209,395 | 153,206,494 | 153,209,395 | CEU | Null genotypes | NA12003 |
| chr10 | 6,659,312 | 6,666,141 | 6,659,312 | 6,666,141 | CEU | Mendelian inconsistencies | (NA12057, NA10851) |
| chr10 | 11,108,870 | 11,112,911 | 11,108,870 | 11,112,911 | YRI | Mendelian inconsistencies | (NA19200, NA19202) |
| chr10 | 20,308,631 | 20,323,198 | 20,315,249 | 20,321,662 | CEU | Mendelian inconsistencies | (NA12760, NA12752) |
| | | | 20,308,631 | 20,323,198 | CEU | Mendelian inconsistencies | (NA12760, NA12752) |
| chr10 | 20,855,214 | 20,859,478 | 20,855,214 | 20,859,478 | CEU | Mendelian inconsistencies | (NA11995, NA10861), (NA12154, NA10830), |
| | | | | | | | (NA07000, NA07029), (NA07056, NA07019), |
| | | | | | | | (NA12239, NA10847), (NA12234, NA10863), |
| | | | | | | | (NA12812, NA12801), (NA12248, NA10835) |
| | | | 20,856,798 | 20,859,015 | YRI | Mendelian inconsistencies | (NA19140, NA19142) |
| | | | 20,855,214 | 20,859,478 | CEU | Hardy-Weinberg | population |
| chr10 | 28,571,813 | 28,573,965 | 28,571,813 | 28,573,965 | YRI | Mendelian inconsistencies | (NA19093, NA19094) |
| chr10 | 37,821,888 | 37,822,922 | 37,821,888 | 37,822,922 | YRI | Mendelian inconsistencies | (NA18501, NA18500), (NA18871, NA18872) |
| chr10 | 41,640,549 | 41,649,682 | 41,640,549 | 41,649,682 | CEU | Mendelian inconsistencies | (NA11829, NA10856), (NA11992, NA10860), |
| | | | | | | | (NA12236, NA10830), (NA12043, NA10857), |
| | | | | | | | (NA12751, NA12740) |
| chr10 | 46,327,384 | 46,342,622 | 46,327,384 | 46,342,622 | YRI | Mendelian inconsistencies | (NA19200, NA19202) |
| chr10 | 46,376,363 | 46,378,186 | 46,376,363 | 46,378,186 | YRI | Mendelian inconsistencies | (NA19093, NA19094) |
| chr10 | 46,993,011 | 47,012,016 | 46,993,011 | 47,012,016 | CEU | Mendelian inconsistencies | (NA11840, NA10854) |
| chr10 | 54,723,271 | 54,798,755 | 54,723,271 | 54,798,755 | YRI | Mendelian inconsistencies | (NA19131, NA19132) |
| chr10 | 57,861,527 | 57,869,790 | 57,861,527 | 57,869,790 | CEU | Mendelian inconsistencies | (NA12815, NA12802) |
| chr10 | 58,435,938 | 58,444,280 | 58,435,938 | 58,444,280 | CEU | Mendelian inconsistencies | (NA07000, NA07029), (NA12717, NA12707), |
| | | | | | | | (NA06993, NA06991), (NA12248, NA10835) |
| chr10 | 65,780,809 | 65,782,243 | 65,780,809 | 65,782,243 | YRI | Mendelian inconsistencies | (NA19099, NA19100) |
| chr10 | 66,655,021 | 66,658,072 | 66,655,021 | 66,658,072 | YRI | Mendelian inconsistencies | (NA19206, NA19208) |
| | | | 66,655,021 | 66,658,072 | JCH | Hardy-Weinberg | population |
| chr10 | 107,333,070 | 107,344,876 | 107,333,070 | 107,344,876 | CEU | Mendelian inconsistencies | (NA06985, NA06991) |
| chr10 | 122,059,897 | 122,066,987 | 122,059,897 | 122,066,987 | YRI | Mendelian inconsistencies | (NA19141, NA19142), (NA19128, NA19129), |
| | | | | | | | (NA19099, NA19100) |
| chr11 | 4,940,386 | 4,941,077 | 4,940,386 | 4,941,077 | YRI | Mendelian inconsistencies | (NA18912, NA18914), (NA19102, NA19103), |
| | | | | | | | (NA19099, NA19100) |
| chr11 | 25,256,148 | 25,258,538 | 25,256,148 | 25,258,538 | YRI | Mendelian inconsistencies | (NA19099, NA19100) |
| chr11 | 49,813,490 | 49,819,016 | 49,813,490 | 49,819,016 | YRI | Null genotypes | NA19099, NA19240 |
| chr11 | 55,147,167 | 55,149,063 | 55,147,167 | 55,149,063 | CEU | Null genotypes | NA12003, NA11832, NA12864, NA12763 |
| chr11 | 108,458,130 | 108,458,972 | 108,458,130 | 108,458,972 | YRI | Mendelian inconsistencies | (NA19098, NA19100) |
| chr12 | 2,117,440 | 2,125,588 | 2,117,440 | 2,125,588 | CEU | Null genotypes | NA12872 |
| chr12 | 3,957,146 | 3,958,803 | 3,957,146 | 3,958,803 | JCH | Null genotypes | NA18547, NA18592, NA18576, NA18976 |
| chr12 | 6,113,877 | 6,116,707 | 6,113,877 | 6,116,707 | CEU | Mendelian inconsistencies | (NA12056, NA10851), (NA12750, NA12740) |
| chr12 | 11,375,250 | 11,435,555 | 11,398,341 | 11,414,113 | CEU | Null genotypes | NA12740 |
| | | | 11,431,147 | 11,435,555 | CEU | Null genotypes | NA12740 |
| | | | 11,398,341 | 11,431,147 | YRI | Null genotypes | NA18502, NA18515, NA18522, NA19238 |
| | | | 11,375,250 | 11,435,555 | CEU | Mendelian inconsistencies | (NA12156, NA10831), (NA12762, NA12753) |
| | | | 11,398,341 | 11,426,356 | CEU | Mendelian inconsistencies | (NA12239, NA10847) |
| | | | 11,402,399 | 11,407,484 | YRI | Mendelian inconsistencies | (NA18508, NA18506), (NA19119, NA19120) |
| | | | 11,400,655 | 11,434,605 | YRI | Mendelian inconsistencies | (NA19119, NA19120) |
| chr12 | 12,031,052 | 12,037,271 | 12,031,052 | 12,037,271 | YRI | Mendelian inconsistencies | (NA18862, NA18863), (NA19207, NA19208), |
| | | | | | | | (NA19143, NA19145) |
| chr12 | 18,456,330 | 18,458,285 | 18,456,330 | 18,458,285 | YRI | Mendelian inconsistencies | (NA19137, NA19139), (NA19159, NA19161) |
| | | | 18,371,121 | 18,456,330 | CEU | Hardy-Weinberg | population |
| chr12 | 21,034,215 | 21,035,483 | 21,034,215 | 21,035,483 | JCH | Null genotypes | NA18594, NA19007 |
| | | | 20,959,604 | 21,054,866 | YRI | Hardy-Weinberg | population |
| chr12 | 22,086,469 | 22,099,211 | 22,086,469 | 22,099,211 | YRI | Mendelian inconsistencies | (NA19141, NA19142) |
| | | | 22,095,425 | 22,125,325 | JCH | Hardy-Weinberg | population |
| chr12 | 22,139,915 | 22,142,636 | 22,139,915 | 22,142,636 | YRI | Mendelian inconsistencies | (NA19200, NA19202), (NA19171, NA19173), |
| | | | | | | | (NA19204, NA19205) |
| chr12 | 27,532,740 | 27,533,809 | 27,532,740 | 27,533,809 | YRI | Mendelian inconsistencies | (NA19171, NA19173) |
| chr12 | 27,539,977 | 27,545,038 | 27,539,977 | 27,543,917 | YRI | Null genotypes | NA18515, NA19160 |
| | | | 27,540,053 | 27,545,038 | YRI | Mendelian inconsistencies | (NA18855, NA18857), (NA19171, NA19173), |
| | | | | | | | (NA19152, NA19154), (NA19143, NA19145), |
| | | | | | | | (NA19099, NA19100), (NA19239, NA19240) |
| chr12 | 30,128,618 | 30,132,410 | 30,128,618 | 30,132,410 | YRI | Null genotypes | NA18856 |
| chr12 | 32,414,722 | 32,422,479 | 32,414,722 | 32,422,479 | YRI | Mendelian inconsistencies | (NA19137, NA19139) |
| chr12 | 33,609,514 | 33,612,689 | 33,609,514 | 33,612,689 | YRI | Mendelian inconsistencies | (NA18871, NA18872), (NA19127, NA19129) |
| chr12 | 33,617,670 | 33,626,050 | 33,617,670 | 33,626,050 | YRI | Mendelian inconsistencies | (NA19098, NA19100) |
| chr12 | 36,397,599 | 36,433,840 | 36,397,599 | 36,433,840 | YRI | Null genotypes | NA18854, NA18853, NA19103, NA19173, |
| | | | | | | | NA19208, NA19132, NA19100 |
| chr12 | 39,104,527 | 39,106,948 | 39,104,527 | 39,106,948 | YRI | Null genotypes | NA19142, NA19140 |
| | | | 39,105,985 | 39,106,948 | YRI | Mendelian inconsistencies | (NA18855, NA18857), (NA19152, NA19154) |
| chr12 | 39,168,293 | 39,169,671 | 39,168,293 | 39,169,671 | JCH | Null genotypes | NA18608, NA18605, NA18594, NA18968, |
| | | | | | | | NA18975 |
| chr12 | 47,011,045 | 47,020,876 | 47,011,045 | 47,020,876 | YRI | Mendelian inconsistencies | (NA18508, NA18506) |
| chr12 | 47,345,081 | 47,357,512 | 47,345,081 | 47,357,512 | YRI | Mendelian inconsistencies | (NA18507, NA18506) |
| chr12 | 50,791,226 | 50,819,085 | 50,791,226 | 50,819,085 | YRI | Mendelian inconsistencies | (NA18505, NA18503) |
| chr12 | 54,001,143 | 54,011,957 | 54,001,143 | 54,011,957 | YRI | Mendelian inconsistencies | (NA18508, NA18506) |
| chr12 | 55,300,111 | 55,301,400 | 55,300,111 | 55,301,400 | CEU | Mendelian inconsistencies | (NA12717, NA12707) |
| chr12 | 55,606,077 | 55,610,763 | 55,606,077 | 55,610,763 | YRI | Null genotypes | NA18503, NA19103 |
| chr12 | 57,502,971 | 57,518,064 | 57,502,971 | 57,518,064 | YRI | Mendelian inconsistencies | (NA18501, NA18500), (NA18507, NA18506), |
| | | | | | | | (NA18870, NA18872), (NA19101, NA19103), |
| | | | | | | | (NA19119, NA19120) |
| chr12 | 58,222,193 | 58,230,898 | 58,222,193 | 58,225,778 | JCH | Null genotypes | NA18965, NA18997, NA18971 |
| | | | 58,225,778 | 58,230,898 | CEU | Mendelian inconsistencies | (NA12155, NA10831), (NA12872, NA12864), |
| | | | | | | | (NA06985, NA06991), (NA12248, NA10835) |
| chr12 | 62,688,136 | 62,690,201 | 62,688,136 | 62,690,201 | YRI | Mendelian inconsistencies | (NA19127, NA19129) |
| chr12 | 63,304,111 | 63,323,750 | 63,304,111 | 63,323,750 | YRI | Mendelian inconsistencies | (NA19143, NA19145) |
| chr12 | 69,160,993 | 69,163,283 | 69,160,993 | 69,163,283 | CEU | Null genotypes | NA10856, NA12751 |
| | | | 69,160,993 | 69,163,283 | CEU | Mendelian inconsistencies | (NA12056, NA10851), (NA12145, NA10846), |
| | | | | | | | (NA12146, NA10847) |
| chr12 | 76,544,928 | 76,569,809 | 76,544,928 | 76,569,809 | YRI | Mendelian inconsistencies | (NA19203, NA19205) |
| chr12 | 78,939,801 | 78,940,386 | 78,939,801 | 78,940,386 | YRI | Mendelian inconsistencies | (NA18522, NA18521) |
| chr12 | 81,459,099 | 81,470,766 | 81,459,099 | 81,470,766 | YRI | Mendelian inconsistencies | (NA19210, NA19211) |
| chr12 | 82,662,983 | 82,669,951 | 82,662,983 | 82,669,951 | JCH | Null genotypes | NA18608, NA18960 |
| chr12 | 86,264,832 | 86,275,499 | 86,264,832 | 86,275,499 | CEU | Mendelian inconsistencies | (NA12003, NA10838), (NA11995, NA10861) |
| chr12 | 88,992,727 | 88,993,412 | 88,992,727 | 88,993,412 | CEU | Mendelian inconsistencies | (NA12006, NA10839), (NA07345, NA07348), |
| | | | | | | | (NA06994, NA07029) |
| | | | 88,992,727 | 88,993,412 | YRI | Mendelian inconsistencies | (NA18858, NA18860) |
| chr12 | 92,363,640 | 92,365,457 | 92,363,640 | 92,365,457 | JCH | Null genotypes | NA18572, NA18563 |
| chr12 | 94,301,826 | 94,311,594 | 94,301,826 | 94,311,594 | YRI | Mendelian inconsistencies | (NA19209, NA19211) |
| chr12 | 96,379,481 | 96,391,285 | 96,379,481 | 96,391,285 | YRI | Mendelian inconsistencies | (NA19138, NA19139) |
| chr12 | 96,517,213 | 96,533,447 | 96,517,213 | 96,533,447 | CEU | Mendelian inconsistencies | (NA12005, NA10839), (NA11831, NA10855) |
| chr12 | 97,173,329 | 97,173,989 | 97,173,329 | 97,173,989 | CEU | Null genotypes | NA12864, NA12760, NA12761, NA12752, |
| | | | | | | | NA12763, NA12753 |
| chr12 | 98,297,960 | 98,304,976 | 98,297,960 | 98,304,976 | CEU | Null genotypes | NA12144 |
| chr12 | 100,026,444 | 100,034,552 | 100,026,444 | 100,034,552 | CEU | Mendelian inconsistencies | (NA06994, NA07029), (NA07056, NA07019), |
| | | | | | | | (NA12249, NA10835) |
| chr12 | 107,297,172 | 107,311,184 | 107,297,172 | 107,311,184 | YRI | Mendelian inconsistencies | (NA18507, NA18506), (NA18522, NA18521), |
| | | | | | | | (NA18912, NA18914), (NA19099, NA19100) |
| chr12 | 109,794,989 | 109,803,752 | 109,794,989 | 109,803,752 | CEU | Mendelian inconsistencies | (NA12751, NA12740) |
| chr12 | 111,568,376 | 111,585,515 | 111,568,376 | 111,585,515 | YRI | Mendelian inconsistencies | (NA18508, NA18506) |
| chr12 | 117,847,706 | 117,853,165 | 117,847,706 | 117,853,165 | JCH | Null genotypes | NA18959, NA18976 |
| chr12 | 124,559,866 | 124,561,057 | 124,559,866 | 124,561,057 | YRI | Mendelian inconsistencies | (NA19131, NA19132) |
| chr12 | 125,947,213 | 125,951,381 | 125,947,213 | 125,951,381 | YRI | Mendelian inconsistencies | (NA18501, NA18500) |
| chr12 | 127,151,101 | 127,159,718 | 127,151,101 | 127,159,718 | YRI | Mendelian inconsistencies | (NA19160, NA19161) |
| chr12 | 129,501,100 | 129,503,209 | 129,501,100 | 129,503,209 | YRI | Mendelian inconsistencies | (NA19203, NA19205) |
| chr12 | 130,083,416 | 130,128,817 | 130,083,416 | 130,128,817 | CEU | Mendelian inconsistencies | (NA07022, NA07019) |
| chr12 | 130,253,222 | 130,299,606 | 130,253,222 | 130,299,606 | YRI | Mendelian inconsistencies | (NA19204, NA19205) |
| chr12 | 130,625,352 | 130,630,204 | 130,625,352 | 130,630,204 | CEU | Null genotypes | NA12006, NA11829, NA10856, NA11994, |
| | | | | | | | NA12154, NA12763, NA12751 |
| | | | 130,625,352 | 130,629,179 | YRI | Mendelian inconsistencies | (NA19201, NA19202), (NA19119, NA19120) |
| chr12 | 131,823,562 | 131,838,245 | 131,823,562 | 131,838,245 | CEU | Null genotypes | NA10838, NA11832, NA11995, NA06985 |
| chr13 | 18,261,867 | 18,268,071 | 18,261,867 | 18,268,071 | YRI | Mendelian inconsistencies | (NA18856, NA18857), (NA19210, NA19211), |
| | | | | | | | (NA19222, NA19221), (NA19099, NA19100) |
| chr13 | 31,939,635 | 31,941,447 | 31,939,635 | 31,941,447 | CEU | Mendelian inconsistencies | (NA12043, NA10857) |
| chr13 | 37,666,773 | 37,668,592 | 37,666,773 | 37,668,592 | CEU | Mendelian inconsistencies | (NA12815, NA12802) |
| chr13 | 46,127,772 | 46,128,419 | 46,127,772 | 46,128,419 | YRI | Mendelian inconsistencies | (NA18504, NA18503) |
| chr13 | 55,553,475 | 55,554,503 | 55,553,475 | 55,554,503 | CEU | Mendelian inconsistencies | (NA11995, NA10861) |
| | | | 55,553,475 | 55,583,831 | YRI | Hardy-Weinberg | population |
| chr13 | 55,559,471 | 55,565,094 | 55,559,471 | 55,565,094 | CEU | Mendelian inconsistencies | (NA11995, NA10861), (NA06985, NA06991) |
| | | | 55,553,475 | 55,583,831 | YRI | Hardy-Weinberg | population |
| chr13 | 65,310,969 | 65,318,345 | 65,310,969 | 65,318,345 | CEU | Mendelian inconsistencies | (NA12043, NA10857) |
| chr13 | 67,050,839 | 67,055,761 | 67,050,839 | 67,055,761 | CEU | Mendelian inconsistencies | (NA11832, NA10855) |
| chr13 | 78,277,405 | 78,301,111 | 78,277,405 | 78,301,111 | CEU | Mendelian inconsistencies | (NA11829, NA10856) |
| chr13 | 95,875,940 | 95,876,926 | 95,875,940 | 95,876,926 | YRI | Mendelian inconsistencies | (NA19201, NA19202) |
| chr13 | 112,751,980 | 112,753,018 | 112,751,980 | 112,753,018 | YRI | Mendelian inconsistencies | (NA18859, NA18860), (NA18861, NA18863) |
| chr14 | 33,107,807 | 33,110,015 | 33,107,807 | 33,110,015 | YRI | Null genotypes | NA18503, NA19171 |
| chr14 | 36,920,093 | 36,928,312 | 36,920,093 | 36,928,312 | YRI | Null genotypes | NA18501, NA18858 |
| chr14 | 68,010,231 | 68,011,603 | 68,010,231 | 68,011,603 | CEU | Null genotypes | NA11832, NA12875, NA06985 |
| chr14 | 74,338,282 | 74,350,474 | 74,338,282 | 74,350,474 | YRI | Null genotypes | NA18502, NA19160, NA19153 |
| chr14 | 104,215,047 | 104,275,522 | 104,215,047 | 104,275,522 | CEU | Null genotypes | NA06994, NA07019 |
| chr14 | 104,485,754 | 104,965,621 | 104,711,227 | 104,741,347 | JCH | Null genotypes | NA18594, NA19000 |
| | | | 104,732,004 | 104,733,584 | YRI | Mendelian inconsistencies | (NA18870, NA18872), (NA19171, NA19173), |
| | | | | | | | (NA19172, NA19173), (NA19159, NA19161) |
| | | | 104,485,754 | 104,965,621 | CEU | Null genotypes | NA07029 |
| | | | 104,873,992 | 104,886,848 | CEU | Null genotypes | NA06994, NA07029 |
| chr15 | 18,840,317 | 18,844,987 | 18,840,317 | 18,844,987 | CEU | Null genotypes | NA11829, NA10856, NA11832, NA07345, |
| | | | | | | | NA12873, NA12874 |
| chr15 | 22,225,104 | 22,261,993 | 22,225,104 | 22,261,993 | CEU | Null genotypes | NA12761, NA11882, NA10835 |
| chr15 | 24,972,760 | 24,980,435 | 24,972,760 | 24,980,435 | CEU | Mendelian inconsistencies | (NA12056, NA10851) |
| chr15 | 32,437,866 | 32,525,037 | 32,437,866 | 32,525,037 | YRI | Null genotypes | NA18500, NA18521, NA19144 |
| chr15 | 54,508,492 | 54,511,694 | 54,508,492 | 54,511,694 | JCH | Null genotypes | NA18561, NA18605 |
| chr17 | 15,244,565 | 15,259,091 | 15,244,565 | 15,259,091 | JCH | Null genotypes | NA18572, NA18592, NA18991 |
| chr17 | 34,186,336 | 34,188,934 | 34,186,336 | 34,188,934 | YRI | Null genotypes | NA18506, NA18914, NA19094, NA19138, |
| | | | | | | | NA19210, NA19161 |
| | | | 34,186,336 | 34,188,934 | YRI | Mendelian inconsistencies | (NA18507, NA18506), (NA19130, NA19132), |
| | | | | | | | (NA19192, NA19194) |
| chr17 | 39,893,166 | 39,898,343 | 39,893,166 | 39,898,343 | YRI | Null genotypes | NA18522, NA19209 |
| chr18 | 1,907,900 | 1,922,838 | 1,907,900 | 1,922,838 | JCH | Null genotypes | NA18635, NA18612 |
| chr18 | 3,815,114 | 3,820,892 | 3,815,114 | 3,820,892 | CEU | Null genotypes | NA12145, NA10846 |
| chr18 | 22,618,475 | 22,619,960 | 22,618,475 | 22,619,960 | YRI | Null genotypes | NA19171 |
| chr18 | 27,285,127 | 27,286,291 | 27,285,468 | 27,286,291 | CEU | Mendelian inconsistencies | (NA11832, NA10855) |
| | | | 27,285,127 | 27,285,713 | CEU | Mendelian inconsistencies | (NA11832, NA10855) |
| chr18 | 29,104,106 | 29,114,504 | 29,104,106 | 29,114,504 | JCH | Null genotypes | NA18624, NA18968, NA19007 |
| chr18 | 32,371,415 | 32,436,184 | 32,371,415 | 32,436,184 | JCH | Null genotypes | NA18971 |
| chr18 | 32,507,103 | 32,508,655 | 32,507,103 | 32,508,655 | YRI | Mendelian inconsistencies | (NA18505, NA18503) |
| chr18 | 32,653,147 | 32,677,786 | 32,653,147 | 32,677,786 | JCH | Null genotypes | NA18971 |
| chr18 | 36,239,519 | 36,247,371 | 36,239,519 | 36,247,371 | YRI | Null genotypes | NA18506 |
| chr18 | 36,512,513 | 36,518,187 | 36,512,513 | 36,518,187 | CEU | Null genotypes | NA10839, NA11993, NA10830, NA06994, |
| | | | | | | | NA12761 |
| chr18 | 36,926,394 | 36,930,790 | 36,926,394 | 36,930,790 | JCH | Null genotypes | NA18571 |
| chr18 | 44,632,511 | 44,636,399 | 44,632,511 | 44,636,399 | JCH | Null genotypes | NA18947, NA18944 |
| chr18 | 45,353,989 | 45,369,896 | 45,353,989 | 45,369,896 | JCH | Null genotypes | NA18947, NA18944 |
| chr18 | 46,252,952 | 46,257,058 | 46,252,952 | 46,257,058 | JCH | Null genotypes | NA18947, NA18944 |
| chr18 | 49,789,714 | 49,796,766 | 49,789,714 | 49,796,766 | YRI | Null genotypes | NA19116 |
| chr18 | 51,289,237 | 51,294,298 | 51,289,237 | 51,294,298 | YRI | Null genotypes | NA19116 |
| chr18 | 56,070,402 | 56,072,449 | 56,070,402 | 56,072,449 | YRI | Mendelian inconsistencies | (NA19140, NA19142) |
| chr18 | 61,350,427 | 61,354,671 | 61,350,427 | 61,354,671 | YRI | Mendelian inconsistencies | (NA19159, NA19161) |
| chr18 | 61,878,056 | 61,879,919 | 61,878,056 | 61,879,919 | YRI | Null genotypes | NA18852, NA19208 |
| chr18 | 64,171,649 | 64,270,532 | 64,171,649 | 64,270,532 | YRI | Null genotypes | NA19100, NA19098 |
| | | | 64,222,744 | 64,261,149 | YRI | Mendelian inconsistencies | (NA19098, NA19100) |
| | | | 64,193,334 | 64,209,977 | YRI | Mendelian inconsistencies | (NA19098, NA19100) |
| | | | 64,241,185 | 64,256,622 | YRI | Mendelian inconsistencies | (NA19098, NA19100) |
| chr18 | 64,444,637 | 64,450,085 | 64,444,637 | 64,450,085 | JCH | Null genotypes | NA18964 |
| chr18 | 64,895,177 | 64,904,477 | 64,895,177 | 64,904,477 | JCH | Null genotypes | NA18564, NA18964, NA18976 |
| chr18 | 69,100,083 | 69,103,448 | 69,100,083 | 69,103,448 | CEU | Mendelian inconsistencies | (NA12812, NA12801) |
| chr18 | 74,392,299 | 74,395,529 | 74,392,299 | 74,395,529 | CEU | Mendelian inconsistencies | (NA12056, NA10851) |
| chr18 | 74,909,007 | 74,923,022 | 74,909,007 | 74,923,022 | JCH | Null genotypes | NA18582 |
| chr19 | 46,046,373 | 46,065,873 | 46,046,373 | 46,065,873 | JCH | Null genotypes | NA18973, NA18952 |
| chr20 | 681,314 | 685,325 | 681,314 | 685,325 | YRI | Mendelian inconsistencies | (NA19153, NA19154) |
| chr20 | 1,564,704 | 1,567,374 | 1,564,704 | 1,567,374 | YRI | Mendelian inconsistencies | (NA18862, NA18863), (NA19160, NA19161), |
| | | | | | | | (NA19130, NA19132), (NA19239, NA19240) |
| chr20 | 6,417,001 | 6,417,570 | 6,417,001 | 6,417,570 | YRI | Mendelian inconsistencies | (NA18862, NA18863), (NA19160, NA19161), |
| | | | | | | | (NA19192, NA19194) |
| chr20 | 14,789,361 | 14,818,472 | 14,789,361 | 14,818,472 | CEU | Mendelian inconsistencies | (NA12043, NA10857) |
| | | | 14,798,454 | 14,817,227 | CEU | Mendelian inconsistencies | (NA12043, NA10857) |
| chr20 | 16,562,202 | 16,580,314 | 16,562,202 | 16,580,314 | YRI | Mendelian inconsistencies | (NA19102, NA19103) |
| chr20 | 41,042,451 | 41,046,282 | 41,042,451 | 41,046,282 | CEU | Mendelian inconsistencies | (NA12155, NA10831) |
| chr20 | 47,638,108 | 47,664,811 | 47,638,108 | 47,664,811 | CEU | Null genotypes | NA07034 |
| chr21 | 9,979,029 | 10,033,456 | 9,979,029 | 10,016,793 | CEU | Null genotypes | NA07357, NA06985, NA12248, NA12249, |
| | | | | | | | NA10835 |
| | | | 10,012,221 | 10,033,456 | JCH | Null genotypes | NA18562 |
| | | | 9,979,029 | 10,012,221 | YRI | Mendelian inconsistencies | (NA19092, NA19094), (NA19172, NA19173), |
| | | | | | | | (NA19140, NA19142) |
| chr21 | 13,483,810 | 13,497,625 | 13,483,810 | 13,497,625 | JCH | Null genotypes | NA18550 |
| chr21 | 13,588,035 | 13,603,210 | 13,588,035 | 13,603,210 | JCH | Null genotypes | NA18550 |
| chr21 | 13,817,281 | 13,833,806 | 13,817,281 | 13,833,806 | JCH | Null genotypes | NA18529 |
| chr21 | 14,003,728 | 14,020,041 | 14,003,728 | 14,020,041 | CEU | Null genotypes | NA10854 |
| | | | 14,003,728 | 14,015,620 | YRI | Null genotypes | NA19161 |
| chr21 | 23,345,578 | 23,354,462 | 23,345,578 | 23,354,462 | YRI | Null genotypes | NA18523 |
| | | | 23,347,306 | 23,352,592 | YRI | Mendelian inconsistencies | (NA18508, NA18506) |
| chr21 | 24,477,775 | 24,500,652 | 24,477,775 | 24,500,652 | JCH | Null genotypes | NA18579 |
| chr21 | 27,117,916 | 27,122,320 | 27,117,916 | 27,122,320 | YRI | Null genotypes | NA19099 |
| chr21 | 32,878,231 | 32,878,744 | 32,878,231 | 32,878,744 | JCH | Null genotypes | NA18945 |
| chr21 | 34,188,732 | 34,190,163 | 34,188,732 | 34,190,163 | CEU | Null genotypes | NA10851, NA12763 |
| chr21 | 34,400,727 | 34,401,819 | 34,400,727 | 34,401,819 | YRI | Null genotypes | NA19098 |
| chr22 | 16,391,062 | 16,393,158 | 16,391,062 | 16,393,158 | JCH | Null genotypes | NA18973, NA18991, NA18994, NA18992, |
| | | | | | | | NA19007 |
| chr22 | 18,146,733 | 18,170,140 | 18,146,733 | 18,170,140 | JCH | Null genotypes | NA18942, NA18973, NA18994, NA18992, |
| | | | | | | | NA19007 |
| chr22 | 19,783,358 | 19,785,828 | 19,783,358 | 19,785,828 | YRI | Mendelian inconsistencies | (NA19210, NA19211) |
| chr22 | 20,705,599 | 20,709,560 | 20,705,599 | 20,709,560 | YRI | Null genotypes | NA19221 |
| chr22 | 20,710,988 | 20,718,781 | 20,710,988 | 20,718,781 | CEU | Mendelian inconsistencies | (NA12716, NA12707) |
| chr22 | 20,751,455 | 20,771,129 | 20,751,455 | 20,771,129 | CEU | Null genotypes | NA12878 |
| | | | 20,751,577 | 20,756,384 | CEU | Mendelian inconsistencies | (NA12716, NA12707) |
| chr22 | 20,779,134 | 20,808,466 | 20,799,464 | 20,808,466 | CEU | Mendelian inconsistencies | (NA12005, NA10839), (NA12716, NA12707) |
| | | | 20,779,134 | 20,806,052 | CEU | Mendelian inconsistencies | (NA12005, NA10839) |
| chr22 | 20,814,462 | 20,826,387 | 20,814,462 | 20,826,387 | CEU | Null genotypes | NA12878 |
| chr22 | 20,829,876 | 20,830,894 | 20,829,876 | 20,830,894 | CEU | Mendelian inconsistencies | (NA12892, NA12878) |
| chr22 | 20,841,759 | 20,858,133 | 20,841,759 | 20,858,133 | JCH | Null genotypes | NA18972 |
| chr22 | 20,861,052 | 20,881,216 | 20,861,052 | 20,881,216 | CEU | Null genotypes | NA12873 |
| chr22 | 21,019,040 | 21,026,845 | 21,019,040 | 21,026,845 | YRI | Null genotypes | NA18523, NA18871 |
| chr22 | 21,026,944 | 21,558,650 | 21,036,340 | 21,046,529 | CEU | Null genotypes | NA10839, NA10846 |
| | | | 21,396,778 | 21,538,141 | CEU | Null genotypes | NA12005, NA10831, NA07357, NA06994, |
| | | | | | | | NA10846, NA12707, NA06985 |
| | | | 21,118,175 | 21,360,293 | CEU | Null genotypes | NA07357, NA06994, NA10846 |
| | | | 21,116,954 | 21,282,684 | CEU | Null genotypes | NA06994, NA10846 |
| | | | 21,104,182 | 21,129,536 | CEU | Null genotypes | NA06994 |
| | | | 21,261,369 | 21,357,002 | CEU | Null genotypes | NA06994 |
| | | | 21,026,944 | 21,069,666 | CEU | Null genotypes | NA10846 |
| | | | 21,247,432 | 21,250,691 | YRI | Null genotypes | NA18523, NA18914, NA19154 |
| | | | 21,037,385 | 21,137,802 | YRI | Null genotypes | NA18523, NA18871, NA19154 |
| | | | 21,221,466 | 21,336,872 | YRI | Null genotypes | NA18523, NA18871, NA19154 |
| | | | 21,036,340 | 21,548,612 | JCH | Null genotypes | NA18526, NA18943, NA18945 |
| | | | 21,223,788 | 21,249,549 | JCH | Null genotypes | NA18972, NA18945 |
| | | | 21,046,432 | 21,110,493 | JCH | Null genotypes | NA18526, NA18972 |
| | | | 21,162,031 | 21,216,889 | JCH | Null genotypes | NA18526, NA18972 |
| | | | 21,240,376 | 21,280,071 | JCH | Null genotypes | NA18526, NA18972 |
| | | | 21,380,867 | 21,548,896 | JCH | Null genotypes | NA18526, NA18972 |
| | | | 21,087,325 | 21,158,310 | JCH | Null genotypes | NA18972 |
| | | | 21,230,945 | 21,240,941 | JCH | Null genotypes | NA18972 |
| | | | 21,396,778 | 21,496,507 | JCH | Null genotypes | NA18972 |
| | | | 21,514,098 | 21,522,758 | JCH | Null genotypes | NA18972 |
| | | | 21,547,040 | 21,558,650 | JCH | Null genotypes | NA18972 |
| | | | 21,193,051 | 21,194,296 | CEU | Mendelian inconsistencies | (NA12005, NA10839), (NA11992, NA10860), |
| | | | | | | | (NA12716, NA12707), (NA12873, NA12864), |
| | | | | | | | (NA07055, NA07048) |
| | | | 21,041,998 | 21,046,529 | CEU | Mendelian inconsistencies | (NA12044, NA10857), (NA12873, NA12864) |
| | | | 21,214,218 | 21,221,815 | CEU | Mendelian inconsistencies | (NA11831, NA10855) |
| | | | 21,238,221 | 21,243,149 | CEU | Mendelian inconsistencies | (NA11993, NA10860) |
| | | | 21,155,789 | 21,163,650 | CEU | Mendelian inconsistencies | (NA12813, NA12801) |
| | | | 21,038,139 | 21,040,959 | CEU | Mendelian inconsistencies | (NA12044, NA10857) |
| | | | 21,268,707 | 21,396,778 | CEU | Mendelian inconsistencies | (NA12044, NA10857) |
| | | | 21,214,620 | 21,235,614 | CEU | Mendelian inconsistencies | (NA07055, NA07048) |
| | | | 21,359,787 | 21,388,825 | YRI | Mendelian inconsistencies | (NA19144, NA19145) |
| | | | 21,247,432 | 21,249,549 | YRI | Mendelian inconsistencies | (NA19140, NA19142) |
| | | | 21,408,825 | 21,411,554 | YRI | Mendelian inconsistencies | (NA19203, NA19205) |
| | | | 21,519,755 | 21,538,141 | YRI | Mendelian inconsistencies | (NA18502, NA18500) |
| | | | 21,046,529 | 21,144,002 | CEU | Hardy-Weinberg | population |
| | | | 21,214,620 | 21,235,614 | CEU | Hardy-Weinberg | population |
| | | | 21,110,493 | 21,131,227 | JCH | Hardy-Weinberg | population |
| chr22 | 22,269,923 | 22,271,906 | 22,269,923 | 22,271,906 | YRI | Null genotypes | NA18503, NA18505 |
| chr22 | 22,701,387 | 22,701,483 | 22,701,387 | 22,701,483 | CEU | Null genotypes (BCM) + | NA10838, NA10851, NA11829, NA10856, |
| | | | | | | Mendelian | NA11832, NA10861, NA07357, NA07056, |
| | | | | | | inconsistencies (Sanger) | NA12043, NA12044, NA10857, NA12239, |
| | | | | | | | NA12874, NA12865, NA12751 |
| chr22 | 24,077,337 | 24,086,564 | 24,077,337 | 24,086,564 | CEU | Mendelian inconsistencies | (NA12716, NA12707) |
| | | | 24,075,427 | 24,095,027 | YRI | Hardy-Weinberg | population |
| chr22 | 24,123,186 | 24,131,503 | 24,123,186 | 24,131,503 | CEU | Mendelian inconsistencies | (NA11830, NA10856) |
| chr22 | 24,230,222 | 24,233,170 | 24,230,222 | 24,233,170 | CEU | Mendelian inconsistencies | (NA12716, NA12707) |
| | | | 24,231,414 | 24,232,995 | YRI | Mendelian inconsistencies | (NA19141, NA19142) |
| chr22 | 28,193,409 | 28,209,121 | 28,193,409 | 28,209,121 | JCH | Null genotypes | NA18552 |
| | | | 28,180,633 | 28,210,597 | JCH | Hardy-Weinberg | population |
| chr22 | 33,621,009 | 33,622,552 | 33,621,009 | 33,622,552 | CEU | Mendelian inconsistencies | (NA12056, NA10851) |
| chr22 | 37,615,466 | 37,624,865 | 37,615,466 | 37,624,865 | CEU | Mendelian inconsistencies | (NA12006, NA10839), (NA12873, NA12864) |
| chr22 | 42,897,001 | 42,911,186 | 42,897,001 | 42,911,186 | JCH | Null genotypes | NA18572 |
| chr22 | 47,865,647 | 47,867,222 | 47,865,647 | 47,867,222 | JCH | Null genotypes | NA18529, NA18944 |

### Other Embodiments

The description of the specific embodiments of the invention is presented for the purposes of illustration. It is not intended to be exhaustive or to limit the scope of the invention to the specific forms described herein. Although the invention has been described with reference to several embodiments, it will be understood by one of ordinary skill in the art that various modifications can be made without departing from the spirit and the scope of the invention, as set forth in the claims. All patents, patent applications, and publications referenced herein are hereby incorporated by reference.

Other embodiments are in the claims.

What is claimed is:

1. A method for predicting the immunocompatibility of the immune system of a first subject with a cell, tissue, or organ from a second subject comprising:
(a) obtaining a first biological sample from the first subject and a second biological sample from the second subject;
(b) determining the presence or absence of at least one deletion variant in the DNA sequence of a gene in the first and second biological samples of step (a), wherein the deletion variant substantially prevents expression of an antigen encoded by the gene, and wherein the at least one deletion variant is in a gene selected from the group consisting of UGT2B28, TRY6, LCE3C, PRB1, OR51A2, ORF4F5, GNB1L, MGAM, and MCEE; and
(c) comparing the presence or absence of the at least one deletion variant determined in step (b) from the first biological sample from the first subject and the second biological sample from the second subject;
wherein the immune system of the first subject is immunocompatible with the cell, tissue, or organ from the second subject if (i) the first subject has at least one intact copy of the gene selected from the group consisting of UGT2B28, TRY6, LCE3C, PRB1, OR51A2, ORF4F5, GNB1L, MGAM, and MCEE, wherein the antigen encoded by the gene is expressed or (ii) the second subject has a deletion variant in all copies of the gene selected from the group consisting of UGT2B28, TRY6, LCE3C, PRB1, OR51A2, ORF4F5, GNB1L, MGAM, and MCEE, wherein said deletion variant substantially prevents expression of the antigen encoded by the gene.

2. The method of claim 1, wherein said first or second biological sample is an organ or part thereof.

3. The method of claim 1, wherein said first or second biological sample is a tissue.

4. The method of claim 1, wherein said first or second biological sample is a bodily fluid.

5. The method of claim 4, wherein said bodily fluid is blood, serum, plasma, bone marrow, cerebrospinal fluid, amniotic fluid, urine, saliva, or semen.

6. The method of claim 1, wherein the presence or absence of the at least one deletion variant is determined by polymerase chain reaction, DNA sequencing, whole-genome sequencing, Southern blotting, restriction fragment length polymorphism analysis, microelectrophoresis, sequencing by hybridization, single molecule sequencing, or microarray analysis.

7. The method of claim 1, wherein the presence or absence of the at least one deletion variant is determined indirectly by genotyping one or more polymorphisms that are in linkage disequilibrium with the deletion variant.

8. The method of claim 7, wherein said polymorphism is a single nucleotide polymorphism (SNP).

9. The method of claim 1, wherein the presence or absence of at least one deletion variant is determined by genotyping one or more polymorphisms that are located inside the sequence that is deleted by the deletion variant.

10. The method of claim 1, wherein the at least one deletion variant is a common deletion variant.

11. The method of claim 1, wherein the at least one deletion variant is at least 100 base pairs in length.

12. The method of claim 1, wherein the at least one deletion variant is in the coding region of the gene.

13. The method of claim 1, wherein the at least one deletion variant is in a regulatory element of the gene.

14. The method of claim 1, wherein the at least one deletion variant is in a gene that is normally expressed in the biological sample.

15. The method of claim 1, wherein the at least one deletion variant is in the UGT2B28 gene.

16. The method of claim 1, comprising determining the presence or absence of at least two deletion variants.

17. The method of claim 1, further comprising determining the blood type or the MHC type for the first and second subjects.

18. The method of claim 1, wherein said second subject is in need of a bone marrow or peripheral blood transplant and said first subject is a potential bone marrow or peripheral blood donor and said method is used to determine if said first subject and said second subject are a donor/recipient match.

19. The method of claim 1, wherein said first subject is a subject in need of an organ or tissue and said second subject is a potential organ or tissue donor and said method is used to determine if said first subject and said second subject are a donor/recipient match.

20. The method of claim 1, wherein said first subject is a woman and said second subject is a prospective father and the method is used to determine if the immune system of the woman is immunocompatible with a sperm from the prospective father.

21. The method of claim 20, wherein said prospective father is a potential sperm donor.

22. The method of claim 1, wherein said first subject is a woman and said second subject is an embryo or fetus.

23. The method of claim 22, wherein the embryo is conceived by in vitro fertilization.

24. The method of claim 22, wherein said antigen is normally expressed by fetal or embryonic cells.

25. The method of claim 1, wherein said second subject is a subject that is in need of a bone marrow or peripheral blood transplant and said first subject is a bone marrow or peripheral blood donor, wherein said method is used to identify said first subject and said second subject as a donor/recipient match if the immune system of the first subject is not immunocompatible with the bone marrow or peripheral blood from the second subject.

26. The method of claim 25, wherein said second subject has a blood cell cancer and wherein said gene encodes an antigen that is specifically expressed on the blood cancer cells.

27. The method of claim 1, further comprising
(d) determining the presence or absence of at least one additional deletion variant in the DNA sequence of a gene in the first and second biological samples of step (a), wherein the deletion variant substantially prevents expression of an antigen encoded by the gene, and wherein the at least one deletion variant is in a gene selected from the group consisting of UGT2B17, GSTT1, GSTM1, and CYP2A6; and
(e) comparing the presence or absence of the at least one additional deletion variant determined in step (d) from the first biological sample from the first subject and the second biological sample from the second subject;
wherein the immune system of the first subject is immunocompatible with the cell, tissue, or organ from the second subject if (i) the first subject has at least one intact copy of the gene selected from the group consisting of UGT2B17, GSTT1, GSTM1, and CYP2A6, wherein the antigen encoded by the gene is expressed or (ii) the second subject has a deletion variant in all copies of the gene selected from the group consisting of UGT2B17, GSTT1, GSTM1, and CYP2A6, wherein said deletion variant substantially prevents expression of the antigen encoded by the gene.

28. The method of claim 27, wherein the at least one deletion variant of step (b) is in the UGT2B28 gene and the at least one additional deletion variant of step (d) is in the UGT2B17 gene.

29. The method of claim 27, comprising determining the presence or absence of at least two additional deletion variants in step (d).

30. The method of claim 29, wherein the at least one deletion variant of step (b) is in the UGT2B28 gene and the at least two additional deletion variants of step (d) are in the GSTM1 and GSTT1 genes.
